SEQUENCE LISTING (65 sequences)

SEQ ID NO: 1 - DNA, 378 bp, Homo sapiens
cgggtgcagctgcaggagtcgggcccaggactggtgaagcctacacagaccctgtccctcacctgcactgtctctggtagctccatcatcagtggaggttacttctggacctggatccgccagcacccagggaagggcctggagtggattgggtacatctattacagtgggagcacctactacagcccgtccctcaagagtcgagttaccatatcagtagacacgtctaagaatcagttctccctgaagctgagctctgtgactgccgcggacacggccgtgtattactgtacgagagcagtaccagctgctaagggttactactcctactacggtatggacgtctggggccaagggaccacggtcaccgtctcctca

SEQ ID NO: 2 - protein, 126 aa, Homo sapiens
RVQLQESGPGLVKPTQTLSLTCTVSGSSIISGGYFWTWIRQHPGKGLEWIGYIYYSGSTYYSPSLKSRVTISVDTSKNQFSLKLSSVTAADTAVYYCTRAVPAAKGYYSYYGMDVWGQGTTVTVSS

SEQ ID NO: 3 - DNA, 336 bp, Homo sapiens
gatattgtgatgactcagtctccactctccctgcccgtcacccctggagagccggcctccatctcctgcaggtctagtcagagcctcctgcatagtaatggatacaactatttggattggttcctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactccattcactttcggccctgggaccaaagtggatatcaaa

SEQ ID NO: 4 - protein, 112 aa, Homo sapiens
DIVMTQSPLSLPVTPGEPASISCRSSQSLLHSNGYNYLDWFLQKPGQSPQLLIYLGSNRASGVPDRFSGSGSGTDFTLKISRVEAEDVGVYYCMQALQTPFTFGPGTKVDIK

SEQ ID NO: 5 - DNA, 378 bp, Homo sapiens
cgggtgcagctgcaggagtcgggcccaggactggtgaagcctacacagaccctgtccctcacctgcactgtctctggtggctccatcatcagtggaggttacttctggacctggatccgccagcacccagggaagggcctggagtggattgggtacatctattacagtgggagcacctactacagcccgtccctcaagagtcgagttaccatatcagtagacacgtctaagaatcagttctccctgaagctgagctctgtgactgccgcggacacggccgtgtattactgtacgagagcagtaccagctgctaagggttactactcctactacggtatggacgtctggggccaagggaccacggtcaccgtctcctca

SEQ ID NO: 6 - protein, 126 aa, Homo sapiens
RVQLQESGPGLVKPTQTLSLTCTVSGGSIISGGYFWTWIRQHPGKGLEWIGYIYYSGSTYYSPSLKSRVTISVDTSKNQFSLKLSSVTAADTAVYYCTRAVPAAKGYYSYYGMDVWGQGTTVTVSS

SEQ ID NO: 7 - DNA, 336 bp, Homo sapiens; identical to SEQ ID NO: 3

SEQ ID NO: 8 - protein, 112 aa, Homo sapiens; identical to SEQ ID NO: 4

SEQ ID NO: 9 - DNA, 378 bp, Homo sapiens; identical to SEQ ID NO: 5

SEQ ID NO: 10 - protein, 126 aa, Homo sapiens; identical to SEQ ID NO: 6

SEQ ID NO: 11 - DNA, 336 bp, Homo sapiens
gatattgtgatgactcagtctccactctccctgcccgtcacccctggagagccggcctccatctcctgcaggtctagtcagagcctcctgcatagtaatggatacaactatttggattggttcctgcagaagccagggcagtctccacagctcctgatctatttgggttctaatcgggcctccggggtccctgacaggttcagtggcagtggatcaggcacagattatacactgaaaatcagcagagtggaggctgaggatgttggggtttattactgcatgcaagctctacaaactccattcactttcggccctgggaccaaagtggatatcaaa

SEQ ID NO: 12 - protein, 112 aa, Homo sapiens
DIVMTQSPLSLPVTPGEPASISCRSSQSLLHSNGYNYLDWFLQKPGQSPQLLIYLGSNRASGVPDRFSGSGSGTDYTLKISRVEAEDVGVYYCMQALQTPFTFGPGTKVDIK

SEQ ID NO: 13 - DNA, 378 bp, Homo sapiens; identical to SEQ ID NO: 5

SEQ ID NO: 14 - protein, 126 aa, Homo sapiens; identical to SEQ ID NO: 6

SEQ ID NO: 15 - DNA, 336 bp, Homo sapiens
gatattgtgatgactcagtctccactctccctgcccgtcacccctggagagccggcctccatctcctgcaggtctagtcagagcctcctgcatagtaatggatacaactatttggattggttcctgcacaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagttctacaaactccattcactttcggccctgggaccaaagtggatatcaaa

SEQ ID NO: 16 - protein, 112 aa, Homo sapiens
DIVMTQSPLSLPVTPGEPASISCRSSQSLLHSNGYNYLDWFLHKPGQSPQLLIYLGSNRASGVPDRFSGSGSGTDFTLKISRVEAEDVGVYYCMQVLQTPFTFGPGTKVDIK

SEQ ID NO: 17 - DNA, 378 bp, Homo sapiens; identical to SEQ ID NO: 5

SEQ ID NO: 18 - protein, 126 aa, Homo sapiens; identical to SEQ ID NO: 6

SEQ ID NO: 19 - DNA, 336 bp, Homo sapiens
gatattgtgatgactcagtctccactctccctgcccgtcacccctggagagccggcctccatctcctgcaggtctagtcagagcctcctgcatagtaatggatacaactatttggattggttcctgcacaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactccattcactttcggccctgggaccaaagtggatatcaaa

SEQ ID NO: 20 - protein, 112 aa, Homo sapiens
DIVMTQSPLSLPVTPGEPASISCRSSQSLLHSNGYNYLDWFLHKPGQSPQLLIYLGSNRASGVPDRFSGSGSGTDFTLKISRVEAEDVGVYYCMQALQTPFTFGPGTKVDIK

SEQ ID NO: 21 - DNA, 378 bp, Homo sapiens
cgggtgcagttgcaggagtcgggcccaggactggtgaagccttcacagaccctgtccctcacctgcactgtctctggtggctccatcatcagtggtggttacttctggacctggatccgccagcacccagggaagggcctggagtggattgggtacatctattacagtgggagcacctactacagcccgtccctcaagagtcgagttaccatatcagtagacacgtctaagaaccagttctccctgaagctgagttctgtgactgccgcggacacggccgtgtattactgtacgagagcagtaccagctgctaagggttactactcctactacggtatggacgtctggggccaagggaccacggtcaccgtctcctca

SEQ ID NO: 22 - protein, 126 aa, Homo sapiens
RVQLQESGPGLVKPSQTLSLTCTVSGGSIISGGYFWTWIRQHPGKGLEWIGYIYYSGSTYYSPSLKSRVTISVDTSKNQFSLKLSSVTAADTAVYYCTRAVPAAKGYYSYYGMDVWGQGTTVTVSS

SEQ ID NO: 23 - DNA, 336 bp, Homo sapiens
gatattgtgatgactcagtctccactctccctgcccgtcacccctggagagccggcctccatctcctgcaggtctagtcagagcctcctgcatagtagtggatacaactatttggattggttcctgcagaagccagggcagtctccacaactcctgatctatttgagttctaatcgggcctccggggtccctgacaggttcagtggcagtggatcaggcacagattttacactgaaaatcagcagagtggaggctgaggatgttggggtttattactgcatgcaagctctacaaactccattcactttcggccctgggaccaaagtggatatcaaa

SEQ ID NO: 24 - protein, 112 aa, Homo sapiens
DIVMTQSPLSLPVTPGEPASISCRSSQSLLHSSGYNYLDWFLQKPGQSPQLLIYLSSNRASGVPDRFSGSGSGTDFTLKISRVEAEDVGVYYCMQALQTPFTFGPGTKVDIK

SEQ ID NO: 25 - protein, 7 aa, Homo sapiens
SGGYFWT

SEQ ID NO: 26 - protein, 16 aa, Homo sapiens
YIYYSGSTYYSPSLKS

SEQ ID NO: 27 - protein, 16 aa, Homo sapiens
AVPAAKGYYSYYGMDV

SEQ ID NO: 28 - protein, 16 aa, Homo sapiens
RSSQSLLHSSGYNYLD

SEQ ID NO: 29 - protein, 7 aa, Homo sapiens
LSSNRAS

SEQ ID NO: 30 - protein, 9 aa, Homo sapiens
MQALQTPFT

SEQ ID NO: 31 - DNA, 360 bp, Homo sapiens
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaactctaacagtggtggcacaaactatgcacagaagtttcagggcagggtcaccatgaccagggacacgtccatcagcacagcctacatggagctgagcaggctgagatctgacgacacggccgtatattactgtgcgagaggacgtgggtatagcagtggctggtatgactactggggccagggaatcctggtcaccgtctcctca

SEQ ID NO: 32 - protein, 120 aa, Homo sapiens
QVQLVQSGAEVKKPGASVKVSCKASGYTFTGYYMHWVRQAPGQGLEWMGWINSNSGGTNYAQKFQGRVTMTRDTSISTAYMELSRLRSDDTAVYYCARGRGYSSGWYDYWGQGILVTVSS

SEQ ID NO: 33 - DNA, 339 bp, Homo sapiens
gacatcgtgatgacccagtctccagactccctggctgtgtctctgggcgagagggccaccatcaactgcaagtccagccagagtgtttttttcagctccagcaatgagaactacttaacttggtaccagcagaaaccaggacagcctcctaaactactcatttactgggcatctacccgggaatccggggtccctgaccgattcagtggcagcgggtctgggacagatttcactctcaccatcagcagcctgcaggctgaagatgtggcagtttattactgtcagcaatattataatattccgtggacgttcggccaagggaccaaggtggaaatcaaa

SEQ ID NO: 34 - protein, 113 aa, Homo sapiens
DIVMTQSPDSLAVSLGERATINCKSSQSVFFSSSNENYLTWYQQKPGQPPKLLIYWASTRESGVPDRFSGSGSGTDFTLTISSLQAEDVAVYYCQQYYNIPWTFGQGTKVEIK

SEQ ID NO: 35 - DNA, 360 bp, Homo sapiens
caagtgcagctggtgcagtctggggttgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactctgcacagaagtttcagggcagggtcaccatgaccagggacacgtccatcagcacagcctacatggagttgagcaggctgagatctgacgacacggccgtgtattactgtgcgagaggacgtgggtataacagtggctggtatgactactggggccagggaaccctggtcaccgtctcctct

SEQ ID NO: 36 - protein, 120 aa, Homo sapiens
QVQLVQSGVEVKKPGASVKVSCKASGYTFTGYYMHWVRQAPGQGLEWMGWINPNSGGTNSAQKFQGRVTMTRDTSISTAYMELSRLRSDDTAVYYCARGRGYNSGWYDYWGQGTLVTVSS

SEQ ID NO: 37 - DNA, 339 bp, Homo sapiens
gacatcgtgatgacccagtccccagactccctggctgtgtctctgggcgagagggccaccatcaactgcaagtccagccagagtgttttacacaactcccacaatgagaactacttaacttggtaccagcagaaaccaggacagcctcctaagctgctcatttactgggcatcttcccgggaatccggggtccctgaccgattcagtggcagcgggtctgggactgatttcactctctccatcagcagcctgcaggctgatgatgtggcagtttattactgtcagcaatatttcagtaatccgtggacgttcggccaagggaccaaggtggaaatcaaa

SEQ ID NO: 38 - protein, 113 aa, Homo sapiens
DIVMTQSPDSLAVSLGERATINCKSSQSVLHNSHNENYLTWYQQKPGQPPKLLIYWASSRESGVPDRFSGSGSGTDFTLSISSLQADDVAVYYCQQYFSNPWTFGQGTKVEIK

SEQ ID NO: 39 - DNA, 360 bp, Homo sapiens
caaatgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaacactaacagtggtggcacaaactgtgcacagaagtttcaggtcagggtcaccatgaccagggacacgtccatcagcacagcctacatggagctgagcaggctgagatctgacgacacggccgtgtattattgtgcgagaggacgcggatatagcagtggctggtacgactactggggccagggaaccctggtcaccgtctcctca

SEQ ID NO: 40 - protein, 120 aa, Homo sapiens
QMQLVQSGAEVKKPGASVKVSCKASGYTFTGYYMHWVRQAPGQGLEWMGWINTNSGGTNCAQKFQVRVTMTRDTSISTAYMELSRLRSDDTAVYYCARGRGYSSGWYDYWGQGTLVTVSS

SEQ ID NO: 41 - DNA, 339 bp, Homo sapiens
gacatcgtgatgacccagtctccagactccctggctgtgtctctgggcgagagggccaccatcaactgcaagtccaaccagaatattttatatagctccagcaatgagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactccgtggacgttcggccaagggaccaaggtggaaatcaaa

SEQ ID NO: 42 - protein, 113 aa, Homo sapiens
DIVMTQSPDSLAVSLGERATINCKSNQNILYSSSNENYLAWYQQKPGQPPKLLIYWASTRESGVPDRFSGSGSGTDFTLTISSLQAEDVAVYYCQQYYSTPWTFGQGTKVEIK

SEQ ID NO: 43 - DNA, 354 bp, Homo sapiens
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagttataaatactatgcagactccgtgcagggccgattcaccatctccagagacaattccaagaacacgctgtatctgcaaatgaacagcctgagagccgaggacacggctgtgtattactgtgcgagagataagggggggcactactttgactactggggccagggaaccctggtcaccgtctcctca

SEQ ID NO: 44 - protein, 118 aa, Homo sapiens
QVQLVESGGGVVQPGRSLRLSCAASGFTFSSYGMHWVRQAPGKGLEWVAVIWYDGSYKYYADSVQGRFTISRDNSKNTLYLQMNSLRAEDTAVYYCARDKGGHYFDYWGQGTLVTVSS

SEQ ID NO: 45 - DNA, 327 bp, Homo sapiens
gacatccagatgacccagtctccatcctccctgtctgcatcagtaggagacagagtcaccatcacttgccgggcaagtcagagcattagcagctatttaaattggtatcagcagaaaccagggaaagcccctaaactcctgatctatgctgcatccagtttgcaaagtggggtcccatcaaggttcagtggcagtggatctgggacagatttcactctcaccatcagcagtctgcaacctgaagattttgcaacttactactgtcaacagaatttcagtttccctccggagtggacgttcggccaagggaccaaggtggaaatcaaa

SEQ ID NO: 46 - protein, 109 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQNFSFPPEWTFGQGTKVEIK

SEQ ID NO: 47 - DNA, 360 bp, Homo sapiens
caggtgcagctggtggagtctgggggaggcgtggtccagcctgggaggtccgtgagactctcctgtgcagcgtctggattcaccttcagtagctatggcatgcactgggtccgccagactccaggcaaggggctggagtgggtggcaattatatggtatgatggaggtattgaatactatgcagactccgtgaagggccgattcaccatctccagagacaattccaagaacacgctgtatctgcaaatgaacagcctgagagccgaggacacggctgtgtattactgtgcgcgagatcacagatatggcagcagctggtttgactactggggccagggaaccctggtcaccgtctcctca

SEQ ID NO: 48 - protein, 120 aa, Homo sapiens
QVQLVESGGGVVQPGRSVRLSCAASGFTFSSYGMHWVRQTPGKGLEWVAIIWYDGGIEYYADSVKGRFTISRDNSKNTLYLQMNSLRAEDTAVYYCARDHRYGSSWFDYWGQGTLVTVSS

SEQ ID NO: 49 - DNA, 321 bp, Homo sapiens
gacatccagatgacccagtctccatcttcactgtctgcatctgtaggagacagagtcaccatcacttgtcgggcgagtcagggcattaacaattatttagcctggtttcagcagaaaccagggaaagcccctaagtccctgatctatgctgcatccagtttgcaaagtggggtcccatcaaatttcagcggcagtggatctgggacagatttcactctctccatcagcagcctgcagcctgaagattttgcaacttattactgccaacagtataaaagttatccgctcactttcggcggagggaccaaggtggagatcaga

SEQ ID NO: 50 - protein, 107 aa, Homo sapiens
DIQMTQSPSSLSASVGDRVTITCRASQGINNYLAWFQQKPGKAPKSLIYAASSLQSGVPSNFSGSGSGTDFTLSISSLQPEDFATYYCQQYKSYPLTFGGGTKVEIR

SEQ ID NO: 51 - protein, 5 aa, Homo sapiens
SYGMH

SEQ ID NO: 52 - protein, 17 aa, Homo sapiens
IIWYDGGIEYYADSVKG

SEQ ID NO: 53 - protein, 11 aa, Homo sapiens
DHRYGSSWFDY

SEQ ID NO: 54 - protein, 11 aa, Homo sapiens
RASQGINNYLA

SEQ ID NO: 55 - protein, 7 aa, Homo sapiens
AASSLQS

SEQ ID NO: 56 - protein, 9 aa, Homo sapiens
QQYKSYPLT

SEQ ID NO: 57 - protein, 124 aa, Homo sapiens
QVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARVVPAAYYYYYGMDVWGQGTTVTVSS

SEQ ID NO: 58 - protein, 118 aa, Homo sapiens
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARGYSSGWYDYWGQGTLVTVSS

SEQ ID NO: 59 - protein, 120 aa, Homo sapiens
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARGRGYSSGWYDYWGQGTLVTVSS

SEQ ID NO: 60 - protein, 117 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDYGGYFDYWGQGTLVTVSS

SEQ ID NO: 61 - protein, 117 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARYSSSWFDYWGQGTLVTVSS

SEQ ID NO: 62 - protein, 112 aa, Homo sapiens
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPFTFGPGTKVDIK

SEQ ID NO: 63 - protein, 113 aa, Homo sapiens
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYNIPWTFGQGTKVEIK

SEQ ID NO: 64 - protein, 113 aa, Homo sapiens; identical to SEQ ID NO: 63

SEQ ID NO: 65 - protein, 107 aa, Homo sapiens
DIQMTQSPSSLSASVGDRVTITCRASQGISNYLAWFQQKPGKAPKSLIYAASSLQSGVPSRFSGSGSGTDFTLTISSLQPEDFATYYCQQYKSYPLTFGGGTKVEIK

(19) United States
(12) Patent Application Publication
(10) Pub. No.: US 2012/0114667 A1
(43) Pub. Date: May 10, 2012

(54) TARGETED BINDING AGENTS DIRECTED TO a5BETA1 AND USES THEREOF

(75) Inventors: Catherine Anne Eberlein, Altrincham Cheshire (GB); Ian Foltz, Burnaby (CA); Jaspal Singh Kang, Surrey (CA); Jane Kendrew, Wilmslow Cheshire (GB); Avril Alfred, Chilliwack (CA); Simon Thomas Barry, Rudyard Staffordshire (GB)

(73) Assignee: MEDIMMUNE LIMITED, Cambridge (GB)

(21) Appl. No.: 13/141,564

(22) PCT Filed: Dec. 21, 2009
(86) PCT No.: PCT/EP09/67706

§ 371 Date: Nov. 14, 2011

Related U.S. Application Data
(60) Provisional application No. 61/140,331, filed on Dec. 23, 2008.

(51) Int. Cl. A61K 39/395; C07K 16/18; C07H 21/04; C12N 15/63; C12N 5/16; C12P 21/06; A61P 35/00; C07K 14/00; A61K 38/00; A61P 29/00; A61P 9/10; A61P 9/00; A61P 5/00; C12N 5/10; C07K 16/00

(52) U.S. Cl. 424/172.1; 530/387.1; 530/388.2; 536/23.53; 435/320.1; 435/332; 435/69.6; 530/350; 514/19.2; 514/19.3; 514/19.4; 514/20.8; 514/16.4; 514/1.4; 536/23.5; 435/358; 435/325

(57) ABSTRACT

The invention relates to targeted binding agents against α5β1 and uses of such agents. More specifically, the invention relates to fully human monoclonal antibodies directed to α5β1. The described targeted binding agents are useful in the treatment of diseases associated with the activity and/or overproduction of α5β1 and as diagnostics.

### BACKGROUND OF THE INVENTION

1. Field of the Invention

The invention relates to targeted binding agents against the target antigen α5β1 integrin (α5β1) and uses of such agents. In some embodiments, the invention relates to fully human monoclonal antibodies directed to α5β1 and uses of these antibodies. Aspects of the invention also relate to hybridomas or other cell lines expressing such targeted binding agents or antibodies. The described targeted binding agents and antibodies are useful as diagnostics and for the treatment of diseases associated with the activity and/or overexpression of α5β1.

2. Description of the Related Art

The integrin superfamily includes at least 24 family members consisting of heterodimers that utilize 18 alpha and 8 beta chains (Hynes, (2002) Cell 110: 673-87). This family of receptors is expressed on the cell surface and mediates cell-cell and cell-extracellular matrix interactions that regulate cell survival, proliferation, migration, and differentiation as well as tumour invasion and metastasis (French-Constant and Colognato, (2004) Trends Cell Biol. 14: 678-86).

Integrins bind to other cellular receptors, growth factors and extracellular matrix proteins, with many family members having overlapping binding specificity for particular proteins. This redundancy may ensure that important functions continue in the absence of a particular integrin (Koivisto et al, (2000) Exp. Cell Res. 255: 10-17). However, temporal and spatial restriction of expression of individual integrins with similar specificity has also been reported and may alter the cellular response to ligand binding (Yokosaki et al, (1996) J. Biol. Chem. 271: 24144-50; Kemperman et al, (1997) Exp. Cell Res. 234: 156-64; Thomas et al, (2006) J. Oral Pathol. Med. 35: 1-10).

The integrin family can be divided into several sub-families based on ligand specificity of the heterodimers. One subfamily consists of all of the integrins that recognize and bind the RGD tripeptide. These receptors include the αIIb/β3 and all of the αV and α5 heterodimers (Thomas et al, (2006) J. Oral Pathol. Med. 35: 1-10). The α5 chain pairs only with the beta 1 chain, although beta 1 is able to pair with a number of other alpha chains. The α5β1 chain heterodimer binds the extracellular matrix component fibronectin as its primary ligand, and has been reported to bind fibrin (Suchiro et al (1997) JBC, 272, 5360-5366) the adhesion molecule L1-CAM (Ruppert et al (1995) JCB, 131, 1881-1891), and to growth factor receptors such as Tie-2 and Flt1 (Cascone et al. (2005) JCB, 170, 993-1004; Orrechia et al (2003) JCS, 116 3479-3489).

Expression of α5 integrin subunit is reported to be ubiquitous at the mRNA level however the level of expression at the level of the protein/receptor varies between tissues and cell types. In addition, it is likely that the integrin is in different “activation” states within these tissues, with “active” α5β1 being associated with active tissue remodeling, or regulation and pathology in the adult. Of particular interest for therapeutics is the function of α5β1 expressed on angiogenic endothelium, macrophages/monocytes, smooth muscle cells, fibroblasts and tumour cells. Expression of α5β1 is often coincident with its major ligand, fibronectin, which forms part of the provisional matrix found in many pathological conditions where vasculature is more permeable, or where tissue damage has occurred. Co-expression of the receptor and ligands is likely to determine the areas where α5β1 is functionally active.

The requirement for α5β1 in vascular remodeling is well established (Watt and Hodivala (1994) Current Biology, 4, 270-272). The α5 Knockout (KO) mice are embryonic lethal due to a failure to form vasculature (Yang et al (1993) Development, 119, 1093-1105). This alone established a pivotal role for α5β1 in vascular remodeling. Consistent with this observation α5β1 function plays a pivotal role in vasculature and embryoid bodies (Francis et al (2002) Arteioscler. Thromb Vasc Biol, 22, 927-933). Knockout of the primary α5β1 ligand, fibronectin, also results in a similar embryonic lethality as a result of a failure to form vasculature. This contrasts with KO of other integrin receptors such as αvβ3, or αvβ5, which do not appear to play the same pivotal role in the angiogenic process. α5β1 expression is specifically upregulated on endothelium in response to various stimuli (Collo and Pepper (1999) JCS, 112, 569-578) and expression of α5β1 in endothelial cells plays a role in promoting expression of genes involved in the regulation of both inflammation and angiogenesis (Klein et al, (2002) MCB, 22, 5912-5922). Consistent with genetic evidence α5β1 appears to be a dominant regulatory integrin in the angiogenic process, when expressed it regulates the activity of other endothelial cell integrins such as α ω̅β3 (Kim et al., (2000) JBC 275, 33920-33928), and suppresses apoptosis. Various antagonists of α5β1 (small molecule, antibody and peptide inhibitors) have been shown to reduce angiogenesis in different in vitro and in vivo systems (Kim et al (2000) Am J Path 156, 1345-1362), confirming the pivotal role in regulating vascular remodeling. Antibodies directed to α5β1 have been disclosed in the following International Patent Applications: WO 1999/58139, WO 2004/056308, WO 2004/089988, WO 2005/092073, WO 2007/134876 and WO 2008/060645.

α5β1 plays a pivotal role in mediating signaling transduction from the extracellular matrix and also regulating signaling from growth factor receptors. Engagement of α5β1 drives actin polymerization, activation of a variety of tyrosine kinases, ERK activation, down regulation of pro-apoptopic drives, and promotes cell cycle progression (Giancotti and Ruoslahti, (1999) Science, 285, 1028-1032). The generic role of α5β1 in signal transduction is consistent with the receptor regulating function of various cell types involved in driving disease pathology. In addition to modulating endothelial cell function, α5β1 is highly expressed on white blood cells is including monocytes and regulates the production of angiogenic chemokines from macrophages (White et al (2001) J. Immunol, 167, 5362-5366). Moreover when engaged to ligand α5β1 regulates survival, cell cycle progress and gene expression in epithelial cells and fibroblasts.

As a result of the pro-survival signalling and transcriptional effects mediated by α5β1, it has also been implicated in promoting survival and growth of tumour cells. In particular α5β1 regulates the growth of astrocytoma (Maglott et al (2006) Can Res 66, 6002-6007) and breast (Jia et al (2004) Can Res, 64, 8674-8681; Spangenberg et al (2006) Can Res, 66, 3715-3725) tumour cells.

Antagonising α5β1 is likely to modulate many processes involved in driving pathologies that involve modified or permeable vasculature, dysfunctional or hyper-proliferative epithelia, including tumour cells, and diseases of chronic inflammation driven by leukocytes.

### SUMMARY OF THE INVENTION

The present invention relates to targeted binding agents that specifically bind to α5β1 and inhibit the growth of cells that express α5β1. Mechanisms by which this can be achieved can include, and are not limited to, blocking ligand binding and/or inhibiting cell signaling implicated in tumour cell growth. The targeted binding agents also inhibit tumour cell adhesion. The targeted binding agents are useful for reducing tumour cell growth and angiogenesis.

In one embodiment of the invention, the targeted binding agent specifically binds to α5β1 integrin with a Kd of less than 100 picomolar (pM). Another embodiment of the invention is a targeted binding agent that specifically binds to α5β1 integrin with a Kd of less than 40 picomolar (pM).

In one embodiment of the invention, the targeted binding agent specifically binds to α5β1 and inhibits binding of fibronectin, fibrin, adhesion molecule L1-CAM, Tie-2 and/or Flt1 ligands to α5β1. Another embodiment of the invention is a targeted binding agent that binds to α5β1 and inhibits downstream cell signaling implicated in cell growth.

In some embodiments, the targeted binding agent binds either the α5 chain or the α5β1 heterodimer and does not cross-react with the β1 chain alone.

Another embodiment of the invention is a targeted binding agent that competes for binding with any of the targeted binding agents or antibodies described herein.

In one embodiment, the targeted binding agent binds α5β1 with a K_{D} of less than about 500 picomolar (pM). In another embodiment, the targeted binding agent binds with a K_{D} less than about 400, 300, 200 or 100 μM. In one embodiment, the targeted binding agent binds with a K_{D} of less than about 75, 60, 50, 40, 30, 20, 10 or 5 μM. Affinity and/or avidity measurements can be measured by FMAT, FACS, and/or BIACORE®, as described herein.

In another embodiment, the targeted binding agent binds α5β1 and with a K_{D} less than about 400, 300, 200, or 100, 75, 60, 50, 40, 30, 20, 10, or 5 μM as measured in a monovalent affinity assay. Monovalent affinity may be measured in a BIACORE® assay in which soluble receptor is flowed over immobilized antibody. In comparison with a bivalent affinity assay, the K_{D} as reported by a monovalent affinity assay is much less likely to be affected by experimental artefacts and is thus able to report a K_{D} much closer to the true monovalent affinity of the antibody. In a bivalent affinity assay, the density of immobilized receptor influences the extent to which single antibody molecules bind twice and/or rebind immobilized receptor as they are flowed over. As such, in a bivalent affinity assay, the density of receptor can directly affect the reported K_{D}. Thus, a monovalent affinity assay provides a much more biologically-relevant measurement of affinity.

In another embodiment, the targeted binding agent inhibits receptor-dependent or ligand-induced signaling with an IC50 less than about 400, 300, 200, or 100, 75, 60, 50, 40, 30, 20, 10, or 5 μM when performed at or close to saturating ligand levels.

In some embodiments of the invention, the targeted binding agent inhibits tumour growth and/or metastasis in a mammal In other embodiments, the targeted binding agent ameliorates symptoms associated with inflammatory disorders in a mammal. In one embodiment, the targeted binding agent ameliorates symptoms associated with inflammatory disorders selected from rheumatoid arthritis or psoriasis in a mammal. Symptoms that may be ameliorated include, but are not limited to, angiogenesis and synovitis. In still other embodiments, the targeted binding agent ameliorates symptoms associated with cardiovascular disease in a mammal Symptoms that may be ameliorated include, but are not limited to, inflammation and angiogenesis. In some other embodiments, the targeted binding agent ameliorates symptoms associated with sepsis in a mammal Symptoms that may be ameliorated include, but are not limited to, uncontrolled vascular permeability, vascular leakage and angiogenesis. In some other embodiments, the targeted binding agent ameliorates symptoms associated with ocular disease. In some other embodiments, the targeted binding agent ameliorates symptoms associated with an ocular disease, such as ischaemic retinopathy or age-related macular degeneration. Symptoms that may be ameliorated include, but are not limited to, uncontrolled vascular permeability and vascular leakage.

In one embodiment of the invention, the targeted binding agent is an antibody. In one embodiment of the invention, the targeted binding agent is a monoclonal antibody. In one embodiment of the invention, the targeted binding agent is a fully human monoclonal antibody. In another embodiment of the invention, the targeted binding agent is a fully human monoclonal antibody of the IgG1, IgG2, IgG3 or IgG4 isotype. In another embodiment of the invention, the targeted binding agent is a fully human monoclonal antibody of the IgG2 isotype. This isotype has reduced potential to elicit effector function in comparison with other isotypes, which may lead to reduced toxicity. In another embodiment of the invention, the targeted binding agent is a fully human monoclonal antibody of the IgG1 isotype. The IgG1 isotype has increased potential to elicit ADCC in comparison with other isotypes, which may lead to improved efficacy. The IgG1 isotype has improved stability in comparison with other isotypes, e.g. IgG4, which may lead to improved bioavailability, or improved ease of manufacture or a longer half-life. In one embodiment, the fully human monoclonal antibody of the IgG1 isotype is of the z, za or f allotype.

In another embodiment the targeted binding agent or antibody may comprise a sequence comprising any one, two, three, four, five or six of the CDR1, CDR2 or CDR3 sequences as shown in Table 12 and/or Table 13. A further embodiment is a targeted binding agent or an antibody that specifically binds to α5β1 and comprises a sequence comprising one of the complementarity determining regions (CDR) sequences shown in Table 12. Embodiments of the invention include a targeted binding agent or antibody comprising a sequence comprising: any one of a CDR1, a CDR2 or a CDR3 sequence as shown in Table 12. A further embodiment is a targeted binding agent or an antibody that specifically binds to α5β1 and comprises a sequence comprising two of the CDR sequences shown in Table 12. In another embodiment the targeted binding agent or antibody comprises a sequence comprising a CDR1, a CDR2 and a CDR3 sequence as shown in Table 12. In another embodiment the targeted binding agent or antibody comprises a sequence comprising one of the CDR sequences shown in Table 13. Embodiments of the invention include a targeted binding agent or antibody comprising a sequence comprising: any one of a CDR1, a CDR2 or a CDR3 sequence as shown in Table 13. In another embodiment the targeted binding agent or antibody comprises a sequence comprising two of the CDR sequences shown in Table 13. In another embodiment the targeted binding agent or antibody comprises a sequence comprising a CDR1, a CDR2 and a CDR3 sequence as shown in Table 13.

In another embodiment the targeted binding agent or antibody may comprise a sequence comprising a CDR1, a CDR2 and a CDR3 sequence as shown in Table 12 and a CDR1, a CDR2 and a CDR3 sequence as shown in Table 13. In some embodiments, the targeted binding agent is an antibody. In certain embodiments, the targeted binding agent is a fully human monoclonal antibody. In certain other embodiments, the targeted binding agent is a binding fragment of a fully human monoclonal antibody. In certain embodiments the antibody is a fully human monoclonal antibody. In certain other embodiments, the targeted binding agent is a binding fragment of a fully human monoclonal antibody.

It is noted that those of ordinary skill in the art can readily accomplish CDR determinations. See for example, Kabat et al., Sequences of Proteins of Immunological Interest, Fifth Edition, NIH Publication 91-3242, Bethesda Md. (1991), vols. 1-3. Kabat provides multiple sequence alignments of immunoglobulin chains from numerous species antibody isotypes. The aligned sequences are numbered according to a single numbering system, the Kabat numbering system. The Kabat sequences have been updated since the 1991 publication and are available as an electronic sequence database (latest downloadable version 1997). Any immunoglobulin sequence can be numbered according to Kabat by performing an alignment with the Kabat reference sequence. Accordingly, the Kabat numbering system provides a uniform system for numbering immunoglobulin chains.

In one embodiment, the targeted binding agent or antibody comprises a sequence comprising any one of the heavy chain sequences shown in Table 12. In another embodiment, the targeted binding agent or antibody comprises a sequence comprising any one of the heavy chain sequences of antibodies 3G11.1A6, 2E10.1B9, 2A9.1A1, 2C5.2B12, 3C2.2A8, 3C5, 8A6.1A3, 2F5.1A4, 9E2.3A8, 7B2.3B10 or 5B11. Light-chain promiscuity is well established in the art, thus, a targeted binding agent or antibody comprising a sequence comprising any one of the heavy chain sequences of antibodies 3G11.1A6, 2E10.1B9, 2A9.1A1, 2C5.2B12, 3C2.2A8, 3C5, 8A6.1A3, 2F5.1A4, 9E2.3A8, 7B2.3B10 or 5B11 or another antibody as disclosed herein, may further comprise any one of the light chain sequences shown in Table 13 or of antibodies 3G11.1A6, 2E10.1B9, 2A9.1A1, 2C5.2B12, 3C2.2A8, 3C5, 8A6.1A3, 2F5.1A4, 9E2.3A8, 7B2.3B10 or 5B11, or another antibody as disclosed herein. In some embodiments, the antibody is a fully human monoclonal antibody.

In one embodiment, the targeted binding agent or antibody comprises a sequence comprising any one of the light chain sequences shown in Table 13. In another embodiment, the targeted binding agent or antibody comprises a sequence comprising any one of the light chain sequences of antibodies 3G11.1A6, 2E10.1B9, 2A9.1A1, 2C5.2B12, 3C2.2A8, 3C5, 8A6.1A3, 2F5.1A4, 9E2.3A8, 7B2.3B10 or 5B11. In some embodiments, the antibody is a fully human monoclonal antibody.

In one embodiment, the targeted binding agent comprises one or more of fully human monoclonal antibodies, 3C5 or 5B11. In certain embodiments, the targeting binding agent is monoclonal antibody 3C5. In certain other embodiments, the targeting binding agent is monoclonal antibody 5B11. In additional embodiments, the targeted binding agent is derivable from any of the foregoing monoclonal antibodies.

In one embodiment a targeted binding agent or an antibody may comprise a sequence comprising a heavy chain CDR1, CDR2 and CDR3 selected from any one of the CDRs of antibodies 3G11.1A6, 2E10.1B9, 2A9.1A1, 2C5.2B12, 3C2.2A8, 3C5, 8A6.1A3, 2F5.1A4, 9E2.3A8, 7B2.3B10 or 5B11. In one embodiment a targeted binding agent or an antibody may comprise a sequence comprising a light chain CDR1, CDR2 and CDR3 selected from any one of the CDRs of antibodies 3G11.1A6, 2E10.1B9, 2A9.1A1, 2C5.2B12, 3C2.2A8, 3C5, 8A6.1A3, 2F5.1A4, 9E2.3A8, 7B2.3B10 or 5B11.

In another embodiment the targeted binding agent or antibody may comprise a sequence comprising any one of a CDR1, a CDR2 or a CDR3 of any one of the fully human monoclonal antibodies 3C5 or 5B11, as shown in Table 12. In another embodiment the targeted binding agent or antibody may comprise a sequence comprising any one of a CDR1, a CDR2 or a CDR3 of any one of the fully human monoclonal antibodies 3C5 or 5B11, as shown in Table 13. In one embodiment the targeted binding agent or antibody may comprise a sequence comprising a CDR1, a CDR2 and a CDR3 of fully human monoclonal antibody 3C5 or 5B11, as shown in Table 12. In another embodiment the targeted binding agent or antibody may comprise a sequence comprising a CDR1, a CDR2 and a CDR3 of fully human monoclonal antibody 3C5 or 5B11, as shown in Table 13. In another embodiment the targeted binding agent or antibody may comprise a sequence comprising a CDR1, a CDR2 and a CDR3 of fully human monoclonal antibody 3C5 or 5B11, as shown in Table 12, and a CDR1, a CDR2 and a CDR3 sequence of fully human monoclonal antibody 3C5 or 5B11, as shown in Table 13. In some embodiments, the antibody is a fully human monoclonal antibody.

In another embodiment the targeted binding agent or antibody may comprise a set of CDRS: HCDR1, HCDR2, HCDR3, LCDR1, LCDR2, LCDR3, wherein the set of CDRS has 10 or fewer amino acid substitutions from a set of CDRs in which:

HCDR1 is amino acid sequence SEQ ID NO:25;

HCDR2 is amino acid sequence SEQ ID NO:26;

HCDR3 is amino acid sequence SEQ ID NO:27;

LCDR1 is amino acid sequence SEQ ID NO:28;

LCDR2 is amino acid sequence SEQ ID NO:29; and

LCDR3 is amino acid sequence SEQ ID NO:30.

In another embodiment the targeted binding agent or antibody may comprise a set of CDRS: HCDR1, HCDR2, HCDR3, LCDR1, LCDR2, LCDR3, wherein the set of CDRS has 10 or fewer amino acid substitutions from a set of CDRs in which:

HCDR1 is amino acid sequence SEQ ID NO:51;

HCDR2 is amino acid sequence SEQ ID NO:52;

HCDR3 is amino acid sequence SEQ ID NO:53;

LCDR1 is amino acid sequence SEQ ID NO:54;

LCDR2 is amino acid sequence SEQ ID NO:55; and

LCDR3 is amino acid sequence SEQ ID NO:56.

A further embodiment of the invention is a targeted binding agent or antibody comprising a sequence comprising the contiguous sequence spanning the framework regions and CDRs, specifically from FR1 through FR4 or CDR1 through CDR3, of any one of the sequences as shown in Table 12 or Table 13. In one embodiment the targeted binding agent or antibody comprises a sequence comprising the contiguous sequences spanning the framework regions and CDRs, specifically from FR1 through FR4 or CDR1 through CDR3, of any one of the sequences of monoclonal antibodies 3C5 or 5B11, as shown in Table 12 or Table 13. In some embodiments, the antibody is a fully human monoclonal antibody.

One embodiment provides a targeted binding agent or antibody, or binding fragment thereof, wherein the agent or antibody, or binding fragment thereof, comprises a heavy chain polypeptide comprising the sequence of SEQ ID NO.:22. In one embodiment, the agent or antibody, or binding fragment thereof, further comprises a light chain polypeptide comprising the sequence of SEQ ID NO.:24. In one embodiment, the targeted binding agent or antibody, or binding fragment thereof, comprises a heavy chain polypeptide comprising the sequence of SEQ ID NO: 22 and a light chain polypeptide comprising the sequence of SEQ ID NO:24. In some embodiments, the antibody is a fully human monoclonal antibody.

In another embodiment the agent or antibody, or binding fragment thereof, comprises a heavy chain polypeptide comprising the sequence of SEQ ID NO.:48. In one embodiment, the agent or antibody, or binding fragment thereof, further comprises a light chain polypeptide comprising the sequence of SEQ ID NO.:50. In one embodiment, the targeted binding agent or antibody, or binding fragment thereof, comprises a heavy chain polypeptide comprising the sequence of SEQ ID NO: 48 and a light chain polypeptide comprising the sequence of SEQ ID NO: 50. In some embodiments, the antibody is a fully human monoclonal antibody.

In one embodiment the targeted binding agent or antibody comprises as many as twenty, sixteen, ten, nine or fewer, e.g. one, two, three, four or five, amino acid additions, substitutions, deletions, and/or insertions within the disclosed CDRs or heavy or light chain sequences. Such modifications may potentially be made at any residue within the CDRs. In some embodiments, the antibody is a fully human monoclonal antibody.

In one embodiment, the targeted binding agent or antibody comprises variants or derivatives of the CDRs disclosed herein, the contiguous sequences spanning the framework regions and CDRs (specifically from FR1 through FR4 or CDR1 through CDR3), the light or heavy chain sequences disclosed herein, or the antibodies disclosed herein. Variants include targeted binding agents or antibodies comprising sequences which have as many as twenty, sixteen, ten, nine or fewer, e.g. one, two, three, four, five or six amino acid additions, substitutions, deletions, and/or insertions in any of the CDR1, CDR2 or CDR3s as shown in Table 12 or Table 13, the contiguous sequences spanning the framework regions and CDRs (specifically from FR1 through FR4 or CDR1 through CDR3) as shown in Table 12 or Table 13, the light or heavy chain sequences disclosed herein, or with the monoclonal antibodies disclosed herein. Variants include targeted binding agents or antibodies comprising sequences which have at least about 60, 70, 80, 85, 90, 95, 98 or about 99% amino acid sequence identity with any of the CDR1, CDR2 or CDR3s as shown in Table 12 or Table 13, the contiguous sequences spanning the framework regions and CDRs (specifically from FR1 through FR4 or CDR1 through CDR3) as shown in Table 12 or Table 13, the light or heavy chain sequences disclosed herein, or with the monoclonal antibodies disclosed herein. The percent identity of two amino acid sequences can be determined by any method known to one skilled in the art, including, but not limited to, pairwise protein alignment. In one embodiment variants comprise changes in the CDR sequences or light or heavy chain polypeptides disclosed herein that are naturally occurring or are introduced by in vitro engineering of native sequences using recombinant DNA techniques or mutagenesis techniques. Naturally occurring variants include those which are generated in vivo in the corresponding germline nucleotide sequences during the generation of an antibody to a foreign antigen. In one embodiment the derivative may be a heteroantibody, that is an antibody in which two or more antibodies are linked together. Derivatives include antibodies which have been chemically modified. Examples include covalent attachment of one or more polymers, such as water-soluble polymers, N-linked, or O-linked carbohydrates, sugars, phosphates, and/or other such molecules. The derivatives are modified in a manner that is different from the naturally occurring or starting antibody, either in the type or location of the molecules attached.

Derivatives further include deletion of one or more chemical groups which are naturally present on the antibody.
In one embodiment, the targeted binding agent is a bispecific antibody. A bispecific antibody is an antibody that has binding specificity for at least two different epitopes. For example, bispecific antibodies can be generated that comprise (i) two antibodies one with a specificity to α5β1 and another to a second molecule that are conjugated together, (ii) a single antibody that has one chain specific to α5β1 and a second chain specific to a second molecule or (iii) a single chain antibody that has specificity to α5β1 and the other molecule. Methods for making bispecific antibodies are known in the art: (See, for example, Millstein et al., Nature, 305:537-539 (1983); Traunecker et al., EMBO J., 10:3655-3659 (1991); Suresh et al., Methods in Enzymology, 121:210 (1986); Kostelny et al., J. Immunol., 148(5):1547-1553 (1992); Hollinger et al., Proc. Natl Acad. Sci. USA, 90:6444-6448 (1993); Gruber et al., J. Immunol., 152:5368 (1994); U.S. Pat. Nos. 4,474,893; 4,714,681; 4,925,648; 5,573,920; 5,601,81; 95,731,168; 4,676,980; and 4,676,980, WO 94/04690; WO 91/00360; WO 92/200373; WO 93/17715; WO 92/08802; and EP 03089). For example, in connection with (i) and (ii) see e.g., Fanger et al. Immunol Methods 4:72-81 (1994) and Wright and Harris, supra. and in connection with (iii) see e.g., Traunecker et al. Int. J. Cancer (Suppl.) 7:51-52 (1992). In each case, the second specificity can be made to the heavy chain activation receptors, including, without limitation, CD16 or CD64 (see e.g., Deo et al. Immunol. Today 18:127 (1997)) or CD89 (see e.g., Valerius et al. Blood 90:4485-4492 (1997)).

In some embodiments of the invention, the targeted binding agent or antibody comprises a sequence comprising SEQ ID NO.: 22. In certain embodiments, SEQ ID NO.: 22 comprises any one of the combinations of germline and non-germline residues indicated by each row of Table 11. In some embodiments, SEQ ID NO: 22 comprises any one, any two, any three, any four, any five, any six, any seven, any eight, any nine or any ten of the germline residues as indicated in Table 11. In other embodiments, the targeted binding agent or antibody is derived from a germline sequence with VH4-31, D2-2 and JH6B domains, wherein one or more residues has been mutated to yield the corresponding germline residue at that position.

In some embodiments of the invention, the targeted binding agent or antibody comprises a sequence comprising SEQ ID NO.: 24. In certain embodiments, SEQ ID NO.: 24 comprises any one of the unique combinations of germline and non-germline residues indicated by each row of Table 10. In some embodiments, SEQ ID NO: 24 comprises any one, any two, any three or all four of the germline residues as indicated in Table 10. In other embodiments, the targeted binding agent or antibody is derived from a germline sequence with A3 and JK3 domains, wherein one or more residues has been mutated to yield the corresponding germline residue at that position.

In some embodiments of the invention, the targeted binding agent or antibody comprises a sequence comprising SEQ ID NO.: 22 and SEQ ID NO: 24. In certain embodiments, SEQ ID NO.: 22 comprises any one of the combinations of germline and non-germline residues indicated by each row of Table 11 and SEQ ID NO:24 comprises any one of the unique combinations of germline and non-germline residues indicated by each row of Table 10. In some embodiments, SEQ ID NO: 22 comprises any one, any two, any three, any four, any five, any six, any seven, any eight, any nine or any ten of the germline residues as indicated in Table 11 and SEQ ID NO: 24 comprises any one, any two, any three or all four of the germline residues as indicated in Table 10.

In some embodiments of the invention, in the targeted binding agent or antibody comprises a sequence comprising SEQ ID NO.: 48. In certain embodiments, SEQ ID NO.: 48 comprises any one of the unique combinations of germline and non-germline residues indicated by each row of Table 9. In some embodiments, SEQ ID NO: 48 comprises any one, any two, any three, any four, any five or all six of the germline residues as indicated in Table 9. In other embodiments, the targeted binding agent or antibody is derived from a germline sequence with VH3-33, D6-13 and JH4B domains, wherein one or more residues has been mutated to yield the corresponding germline residue at that position.

In some embodiments of the invention, the targeted binding agent or antibody comprises a sequence comprising SEQ ID NO.: 50. In certain embodiments, SEQ ID NO.: 50 comprises any one of the unique combinations of germline and non-germline residues indicated by each row of Table 8. In some embodiments, SEQ ID NO: 50 comprises any one, any two, any three or all four of the germline residues as indicated in Table 8. In certain embodiments, the targeted binding agent or antibody is derived from a germline sequence with L1 and JK4 domains, wherein one or more residues has been mutated to yield the corresponding germline residue at that position.

In some embodiments of the invention, in the targeted binding agent or antibody comprises a sequence comprising SEQ ID NO.: 48. and SEQ ID NO:50. In certain embodiments, SEQ ID NO.: 48 comprises any one of the unique combinations of germline and non-germline residues indicated by each row of Table 9 and SEQ ID NO.: 50 comprises any one of the unique combinations of germline and non-germline residues indicated by each row of Table 8. In some embodiments, SEQ ID NO: 48 comprises any one, any two, any three, any four, any five or all six of the germline residues as indicated in Table 9 and SEQ ID NO: 50 comprises any one, any two, any three or all four of the germline residues as indicated in Table 8.

A further embodiment of the invention is a targeted binding agent or antibody which competes or cross-competes for binding to α5β1 with the targeted binding agent or antibodies of the invention. In another embodiment of the invention there is an antibody which competes or cross-competes for binding to α5β1 with the targeted binding agent or antibodies of the invention. In another embodiment the targeted binding agent or antibody competes for binding to α5β1 with any one of fully human monoclonal antibodies 3C5 or 5B11. “Competes” indicates that the targeted binding agent or antibody competes for binding to α5β1 with any one of fully human monoclonal antibodies 3C5 or 5B11, i.e. competition is unidirectional.

Embodiments of the invention include a targeted binding agent or antibody which cross competes with any one of fully human monoclonal antibodies 3C5 or 5B11 for binding to α5β1. “Cross competes” indicates that the targeted binding agent or antibody competes for binding to α5β1 with any one of fully human monoclonal antibodies 3C5 or 5B11, and vice versa, i.e. competition is bidirectional.

A further embodiment of the invention is a targeted binding agent or antibody that binds to the same epitope on α5β1 as the targeted binding agent or antibodies of the invention. Embodiments of the invention also include a targeted binding agent or antibody that binds to the same epitope on α5β1 as any one of fully human monoclonal antibodies 3C5 or 5B11. Other embodiments of the invention include isolated nucleic acid molecules encoding any of the targeted binding agents or antibodies described herein, vectors having isolated nucleic acid molecules encoding the targeted binding agents or antibodies described herein or a host cell transformed with any of such nucleic acid molecules. Embodiments of the invention include a nucleic acid molecule encoding a fully human isolated targeted binding agent that specifically bind to α5β1 and inhibit binding of fibronectin, fibrin, adhesion molecule L1-CAM and growth factor receptors such as Tie-2 and Flt1 to α5β1. The invention also encompasses polynucleotides that hybridize under stringent or lower stringency hybridization conditions, as defined herein, to polynucleotides that encode any of the targeted binding agents or antibodies described herein.

Embodiments of the invention described herein also provide cells for producing these antibodies. Examples of cells include hybridomas, or recombinantly created cells, such as Chinese hamster ovary (CHO) cells, variants of CHO cells (for example DG44) and NSO cells that produce antibodies against α5β1. Additional information about variants of CHO cells can be found in Andersen and Reilly (2004) Current Opinion in Biotechnology 15, 456-462 which is incorporated herein in its entirety by reference. The antibody can be manufactured from a hybridoma that secretes the antibody, or from a recombinantly engineered cell that has been transformed or transfected with a gene or genes encoding the antibody.

In addition, one embodiment of the invention is a method of producing an antibody of the invention by culturing host cells under conditions wherein a nucleic acid molecule is expressed to produce the antibody followed by recovering the antibody. It should be realised that embodiments of the invention also include any nucleic acid molecule which encodes an antibody or fragment of an antibody of the invention including nucleic acid sequences optimised for increasing yields of antibodies or fragments thereof when transfected into host cells for antibody production.

A further embodiment herein includes a method of producing antibodies that specifically bind to α5β1 and inhibit the biological activity of α5β1, by immunising a mammal with cells expressing human α5β1, isolated cell membranes containing human α5β1, purified human α5β1, or a fragment thereof, and/or one or more orthologous sequences or fragments thereof.

In other embodiments the invention provides compositions, including a targeted binding agent or antibody of the invention or binding fragment thereof, and a pharmaceutically acceptable carrier or diluent.

Still further embodiments of the invention include methods of treating an animal suffering from a neoplastic disease by administering to the animal a therapeutically effective dose of a targeted binding agent that specifically binds to α5β1. In certain embodiments the method further comprises selecting an animal in need of treatment for a neoplastic disease, and administering to the animal a therapeutically effective dose of a targeted binding agent that specifically binds to α5β1.

Still further embodiments of the invention include methods of treating an animal suffering from a non-neoplastic disease by administering to the animal a therapeutically effective dose of a targeted binding agent that specifically binds to α5β1. In certain embodiments the method further comprises selecting an animal in need of treatment for a non-neoplastic disease, and administering to the animal a therapeutically effective dose of a targeted binding agent that specifically binds to α5β1.

Still further embodiments of the invention include methods of treating an animal suffering from a malignant tumour by administering to the animal a therapeutically effective dose of a targeted binding agent that specifically binds to α5β1. In certain embodiments the method further comprises selecting an animal in need of treatment for a malignant tumour, and administering to the animal a therapeutically effective dose of a targeted binding agent that specifically binds to α5β1.

Still further embodiments of the invention include methods of treating an animal suffering from an inflammatory disorder by administering to the animal a therapeutically effective dose of a targeted binding agent that specifically binds to α5β1. In certain embodiments the method further comprises selecting an animal in need of treatment for an inflammatory disorder, and administering to the animal a therapeutically effective dose of a targeted binding agent that specifically binds to α5β1.

Still further embodiments of the invention include methods of treating an animal suffering from a disease or condition associated with α5β1 expression by administering to the animal a therapeutically effective dose of a targeted binding agent that specifically binds to α5β1. In certain embodiments the method further comprises selecting an animal in need of treatment for a disease or condition associated with α5β1 expression, and administering to the animal a therapeutically effective dose of a targeted binding agent that specifically binds to α5β1.

A malignant tumour may be selected from the group consisting of: melanoma, small cell lung cancer, non-small cell lung cancer, glioma, hepatocellular (liver) carcinoma, thyroid tumour, gastric (stomach) cancer, prostate cancer, breast cancer, ovarian cancer, bladder cancer, lung cancer, glioblastoma, endometrial cancer, kidney cancer, colon cancer, pancreatic cancer, esophageal carcinoma, head and neck cancers, mesothelioma, sarcomas, biliary (cholangiocarcinoma), small bowel adenocarcinoma, pediatric malignancies and epidermoid carcinoma.

Treatable proliferative, angiogenic, cell adhesion or invasion-related diseases include neoplastic diseases, such as, melanoma, small cell lung cancer, non-small cell lung cancer, glioma, hepatocellular (liver) carcinoma, thyroid tumour, gastric (stomach) cancer, gallbladder cancer, prostate cancer, breast cancer, ovarian cancer, bladder cancer, lung cancer, glioblastoma, endometrial cancer, kidney cancer, colon cancer, pancreatic cancer, esophageal carcinoma, head and neck cancers, mesothelioma, sarcomas, biliary (cholangiocarcinoma), small bowel adenocarcinoma, pediatric malignancies, epidermoid carcinoma and leukaemia, including chronic myelogenous leukaemia.

In one embodiment, the neoplastic disease is melanoma, colon cancer or chronic myelogenous leukaemia.

Non-neoplastic diseases include inflammatory disorders such as rheumatoid arthritis or psoriasis, cardiovascular disease such as atherosclerosis, sepsis, ocular disease such as ischaemic retinopathy or age-related macular degeneration (AMD).

Inflammatory disorders include rheumatoid arthritis, osteoarthritis, asthma, chronic obstructive pulmonary disease (COPD), allergic rhinitis and psoriasis.

In one embodiment the present invention is suitable for use in inhibiting α5β1, in patients with a tumour which is dependent alone, or in part, on α5β1.

Still further embodiments of the invention include use of a targeted binding agent or antibody of the invention in the preparation of a medicament for the treatment of an animal suffering from a proliferative, angiogenic, cell adhesion or invasion-related disease. In certain embodiments the use further comprises selecting an animal in need of treatment for a proliferative, angiogenic, cell adhesion or invasion-related disease.

Still further embodiments of the invention include use of a targeted binding agent or antibody of the invention in the preparation of a medicament for the treatment of an animal suffering from a neoplastic disease. In certain embodiments the use further comprises selecting an animal in need of treatment for a neoplastic disease.

Still further embodiments of the invention include use of a targeted binding agent or antibody of the invention in the preparation of a medicament for the treatment of an animal suffering from a non-neoplastic disease. In certain embodiments the use further comprises selecting an animal in need of treatment for a non-neoplastic disease.

Still further embodiments of the invention include use of a targeted binding agent or antibody of the invention in the preparation of a medicament for the treatment of an animal suffering from a malignant tumour. In certain embodiments the use further comprises selecting an animal in need of treatment for a malignant tumour.

Still further embodiments of the invention include use of a targeted binding agent or antibody of the invention in the preparation of a medicament for the treatment of an animal suffering from an inflammatory disease. In certain embodiments the use further comprises selecting an animal in need of treatment for an inflammatory disease.

Still further embodiments of the invention include use of a targeted binding agent or antibody of the invention in the preparation of a medicament for the treatment of an animal suffering from a disease or condition associated with α5β1 expression. In certain embodiments the use further comprises selecting an animal in need of treatment for a disease or condition associated with α5β1 expression.

Still further embodiments of the invention include a targeted binding agent or antibody of the invention for the treatment of an animal suffering from a proliferative, angiogenic, cell adhesion or invasion-related disease.

Still further embodiments of the invention include a targeted binding agent or antibody of the invention for the treatment of an animal suffering from a neoplastic disease.

Still further embodiments of the invention include a targeted binding agent or antibody of the invention for the treatment of an animal suffering from a non-neoplastic disease.

Still further embodiments of the invention include a targeted binding agent or antibody of the invention for the treatment of an animal suffering from a malignant tumour.

Still further embodiments of the invention include a targeted binding agent or antibody of the invention for the treatment of an animal suffering from a disease or condition associated with α5β1 expression.

In one embodiment treatment of a
a proliferative, angiogenic, cell adhesion or invasion-related disease;
a neoplastic disease;
a non-neoplastic disease;
a malignant tumour;
an inflammatory disorder; or
a disease or condition associated with α5β1 expression

comprises managing, ameliorating, preventing, any of the aforementioned diseases or conditions.

In one embodiment treatment of a neoplastic disease comprises inhibition of tumour growth, tumour growth delay, regression of tumour, shrinkage of tumour, increased time to regrowth of tumour on cessation of treatment, increased time to tumour recurrence, slowing of disease progression.

In some embodiments of the invention, the animal to be treated is a human

In some embodiments of the invention, the targeted binding agent is a fully human monoclonal antibody.

In some embodiments of the invention, the targeted binding agent is selected from the group consisting of fully human monoclonal antibodies 3C5 or 5B11.

Embodiments of the invention include a conjugate comprising the targeted binding agent as described herein, and a therapeutic agent. In some embodiments of the invention, the therapeutic agent is a toxin. In other embodiments, the therapeutic agent is a radioisotope. In still other embodiments, the therapeutic agent is a pharmaceutical composition.

In another aspect, a method of selectively killing a cancerous cell in a patient is provided. The method comprises administering a fully human antibody conjugate to a patient. The fully human antibody conjugate comprises an antibody that can bind to α5β1 and an agent. The agent is either a toxin, a radioisotope, or another substance that will kill a cancer cell. The antibody conjugate thereby selectively kills the cancer cell.

In one aspect, a conjugated fully human antibody that specifically binds to α5β1 is provided. Attached to the antibody is an agent, and the binding of the antibody to a cell results in the delivery of the agent to the cell. In one embodiment, the above conjugated fully human antibody binds to an extracellular domain of α5β1. In another embodiment, the antibody and conjugated toxin are internalised by a cell that expresses α5β1. In another embodiment, the agent is a cytotoxic agent. In another embodiment, the agent is, for example saporin, or auristatin, pseudomonas exotoxin, gelonin, ricin, calicheamicin or maytansine-based immunoconjugates, and the like. In still another embodiment, the agent is a radioisotope.

The targeted binding agent or antibody of the invention can be administered alone, or can be administered in combination with additional antibodies or chemotherapeutic drugs or radiation therapy. For example, a monoclonal, oligoclonal or polyclonal mixture of α5β1 antibodies that block cell adhesion, invasion, angiogenesis or proliferation can be administered in combination with a drug shown to inhibit tumour cell proliferation.

Another embodiment of the invention includes a method of diagnosing diseases or conditions in which an antibody as disclosed herein is utilised to detect the level of α5β1 in a patient or patient sample. In one embodiment, the patient sample is blood or blood serum or urine. In further embodiments, methods for the identification of risk factors, diagnosis of disease, and staging of disease is presented which involves the identification of the expression and/or overexpression of α5β1 using anti-α5β1 antibodies. In some embodiments, the methods comprise administering to a patient a fully human antibody conjugate that selectively binds to α5β1 on a cell. The antibody conjugate comprises an antibody that specifically binds to α5β1 and a label. The methods further comprise observing the presence of the label in the patient. A relatively high amount of the label on specific cell types will indicate a relatively high risk of the disease and a relatively low amount of the label will indicate a relatively low risk of the disease. In one embodiment, the label is a green fluorescent protein.

The invention further provides methods for assaying the level of α5β1 in a patient sample, comprising contacting an antibody as disclosed herein with a biological sample from a patient, and detecting the level of binding between said antibody and α5β1 in said sample. In more specific embodiments, the biological sample is blood, plasma or serum.

Another embodiment of the invention includes a method for diagnosing a condition associated with the expression of α5β1 in a cell by contacting the serum or a cell with an antibody as disclosed herein, and thereafter detecting the presence of α5β1. In one embodiment the condition can be a proliferative, angiogenic, cell adhesion or invasion-related disease including, but not limited to, a neoplastic disease.

In another embodiment, the invention includes an assay kit for detecting α5β1 in mammalian tissues, cells, or body fluids to screen for α5β1-related diseases. The kit includes an antibody as disclosed herein and a means for indicating the reaction of the antibody with α5β1, if present. In one embodiment the antibody is a monoclonal antibody. In one embodiment, the antibody that binds α5β1 is labelled. In another embodiment the antibody is an unlabelled primary antibody and the kit further includes a means for detecting the primary antibody. In one embodiment, the means for detecting includes a labelled second antibody that is an anti-immunoglobulin. The antibody may be labelled with a marker selected from the group consisting of a fluorochrome, an enzyme, a radionuclide and a radiopaque material.

In some embodiments, the targeted binding agents or antibodies as disclosed herein can be modified to enhance their capability of fixing complement and participating in complement-dependent cytotoxicity (CDC). In other embodiments, the targeted binding agents or antibodies can be modified to enhance their capability of activating effector cells and participating in antibody-dependent cytotoxicity (ADCC). In yet other embodiments, the targeted binding agents or antibodies as disclosed herein can be modified both to enhance their capability of activating effector cells and participating in antibody-dependent cytotoxicity (ADCC) and to enhance their capability of fixing complement and participating in complement-dependent cytotoxicity (CDC).

In some embodiments, the targeted binding agents or antibodies as disclosed herein can be modified to reduce their capability of fixing complement and participating in complement-dependent cytotoxicity (CDC). In other embodiments, the targeted binding agents or antibodies can be modified to reduce their capability of activating effector cells and participating in antibody-dependent cytotoxicity (ADCC). In yet other embodiments, the targeted binding agents or antibodies as disclosed herein can be modified both to reduce their capability of activating effector cells and participating in antibody-dependent cytotoxicity (ADCC) and to reduce their capability of fixing complement and participating in complement-dependent cytotoxicity (CDC).

In certain embodiments, the half-life of a targeted binding agent or antibody as disclosed herein and of compositions of the invention is at least about 4 to 7 days. In certain embodiments, the mean half-life of a targeted binding agent or antibody as disclosed herein and of compositions of the invention is at least about 2 to 5 days, 3 to 6 days, 4 to 7 days, 5 to 8 days, 6 to 9 days, 7 to 10 days, 8 to 11 days, 8 to 12, 9 to 13, 10 to 14, 11 to 15, 12 to 16, 13 to 17, 14 to 18, 15 to 19, or 16 to 20 days. In other embodiments, the mean half-life of a targeted binding agent or antibody as disclosed herein and of compositions of the invention is at least about 17 to 21 days, 18 to 22 days, 19 to 23 days, 20 to 24 days, 21 to 25, days, 22 to 26 days, 23 to 27 days, 24 to 28 days, 25 to 29 days, or 26 to 30 days. In still further embodiments the half-life of a targeted binding agent or antibody as disclosed herein and of compositions of the invention can be up to about 50 days. In certain embodiments, the half-lives of antibodies and of compositions of the invention can be prolonged by methods known in the art. Such prolongation can in turn reduce the amount and/or frequency of dosing of the antibody compositions. Antibodies with improved in vivo half-lives and methods for preparing them are disclosed in U.S. Pat. No. 6,277,375; and International Publication Nos. WO 98/23289 and WO 97/3461.

In another embodiment, the invention provides an article of manufacture including a container. The container includes a composition containing a targeted binding agent or antibody as disclosed herein, and a package insert or label indicating that the composition can be used to treat cell adhesion, invasion, angiogenesis, and/or proliferation-related diseases, including, but not limited to, diseases characterised by the expression or overexpression of α5β1.

In other embodiments, the invention provides a kit comprising a composition containing a targeted binding agent or antibody as disclosed herein, and instructions to administer the composition to a subject in need of treatment.

The present invention provides formulation of proteins comprising a variant Fc region. That is, a non-naturally occurring Fc region, for example an Fc region comprising one or more non-naturally occurring amino acid residues, i.e. an amino acid other than the amino acid normally found at a particular position. Also encompassed by the variant Fc regions of present invention are Fc regions which comprise amino acid deletions, additions and/or modifications.

The serum half-life of proteins comprising Fc regions may be increased by increasing the binding affinity of the Fc region for FcRn. In one embodiment, the Fc variant protein has enhanced serum half life relative to comparable molecule.

In another embodiment, the present invention provides an Fc variant, wherein the Fc region comprises at least one non naturally occurring amino acid at one or more positions selected from the group consisting of 239, 330 and 332, as numbered by the EU index as set forth in Kabat. In a specific embodiment, the present invention provides an Fc variant, wherein the Fc region comprises at least one non naturally occurring amino acid selected from the group consisting of 239D, 330L and 332E, as numbered by the EU index as set forth in Kabat. Optionally, the Fc region may further comprise additional non-naturally occurring amino acid at one or more positions selected from the group consisting of 252, 254, and 256, as numbered by the EU index as set forth in Kabat. In a specific embodiment, the present invention provides an Fc variant, wherein the Fc region comprises at least one non naturally occurring amino acid selected from the group consisting of 239D, 330L and 332E, as numbered by the EU index as set forth in Kabat and at least one non naturally occurring amino acid at one or more positions selected from the group consisting of 252Y, 254T and 256E, as numbered by the EU index as set forth in Kabat.

In another embodiment, the present invention provides an Fc variant, wherein the Fc region comprises at least one non naturally occurring amino acid at one or more positions selected from the group consisting of 234, 235 and 331, as numbered by the EU index as set forth in Kabat. In a specific embodiment, the present invention provides an Fc variant, wherein the Fc region comprises at least one non naturally occurring amino acid selected from the group consisting of 234F, 235F, 235Y, and 331S, as numbered by the EU index as set forth in Kabat. In a further specific embodiment, an Fc variant of the invention comprises the 234F, 235F, and 331S non naturally occurring amino acid residues, as numbered by the EU index as set forth in Kabat. In another specific embodiment, an Fc variant of the invention comprises the 234F, 235Y, and 331S non naturally occurring amino acid residues, as numbered by the EU index as set forth in Kabat. Optionally, the Fc region may further comprise additional non naturally occurring amino acid at one or more positions selected from the group consisting of 252, 254, and 256, as numbered by the EU index as set forth in Kabat. In a specific embodiment, the present invention provides an Fc variant, wherein the Fc region comprises at least one non naturally occurring amino acid selected from the group consisting of 234F, 235F, 235Y, and 331S, as numbered by the EU index as set forth in Kabat; and at least one non naturally occurring amino acid at one or more positions are selected from the group consisting of 252Y, 254T and 256E, as numbered by the EU index as set forth in Kabat.

In another embodiment, the present invention provides an Fc variant protein formulation, wherein the Fc region comprises at least a non naturally occurring amino acid at one or more positions selected from the group consisting of 239, 330 and 332, as numbered by the EU index as set forth in Kabat. In a specific embodiment, the present invention provides an Fc variant protein formulation, wherein the Fc region comprises at least one non naturally occurring amino acid selected from the group consisting of 239D, 330L and 332E, as numbered by the EU index as set forth in Kabat. Optionally, the Fc region may further comprise additional non naturally occurring amino acid at one or more positions selected from the group consisting of 252, 254, and 256, as numbered by the EU index as set forth in Kabat. In a specific embodiment, the present invention provides an Fc variant protein formulation, wherein the Fc region comprises at least one non naturally occurring amino acid selected from the group consisting of 239D, 330L and 332E, as numbered by the EU index as set forth in Kabat and at least one non naturally occurring amino acid at one or more positions are selected from the group consisting of 252Y, 254T and 256E, as numbered by the EU index as set forth in Kabat.

In another embodiment, the present invention provides an Fc variant protein formulation, wherein the Fc region comprises at least one non naturally occurring amino acid at one or more positions selected from the group consisting of 234, 235 and 331, as numbered by the EU index as set forth in Kabat. In a specific embodiment, the present invention provides an Fc variant protein formulation, wherein the Fc region comprises at least one non naturally occurring amino acid selected from the group consisting of 234F, 235F, 235Y, and 331S, as numbered by the EU index as set forth in Kabat. Optionally, the Fc region may further comprise additional non naturally occurring amino acid at one or more positions selected from the group consisting of 252, 254, and 256, as numbered by the EU index as set forth in Kabat. In a specific embodiment, the present invention provides an Fc variant protein formulation, wherein the Fc region comprises at least one non naturally occurring amino acid selected from the group consisting of 234F, 235F, 235Y, and 331S, as numbered by the EU index as set forth in Kabat; and at least one non naturally occurring amino acid at one or more positions are selected from the group consisting of 252Y, 254T and 256E, as numbered by the EU index as set forth in Kabat.

Methods for generating non naturally occurring Fc regions are known in the art. For example, amino acid substitutions and/or deletions can be generated by mutagenesis methods, including, but not limited to, site-directed mutagenesis (Kunkel, Proc. Natl. Acad. Sci. USA 82:488-492 (1985)), PCR mutagenesis (Higuchi, in “PCR Protocols: A Guide to Methods and Applications”, Academic Press, San Diego, pp. 177-183 (1990)), and cassette mutagenesis (Wells et al., Gene 34:315-323 (1985)). Preferably, site-directed mutagenesis is performed by the overlap-extension PCR method (Higuchi, in “PCR Technology: Principles and Applications for DNA Amplification”, Stockton Press, New York, pp. 61-70 (1989)). The technique of overlap-extension PCR (Higuchi, ibid.) can also be used to introduce any desired mutation(s) into a target sequence (the starting DNA). For example, the first round of PCR in the overlap-extension method involves amplifying the target sequence with an outside primer (primer 1) and an internal mutagenesis primer (primer 3), and separately with a second outside primer (primer 4) and an internal primer (primer 2), yielding two PCR segments (segments A and B). The internal mutagenesis primer (primer 3) is designed to contain mismatches to the target sequence specifying the desired mutation(s). In the second round of PCR, the products of the first round of PCR (segments A and B) are amplified by PCR using the two outside primers (primers 1 and 4). The resulting full-length PCR segment (segment C) is digested with restriction enzymes and the resulting restriction fragment is cloned into an appropriate vector. As the first step of mutagenesis, the starting DNA (e.g., encoding an Fc fusion protein, an antibody or simply an Fc region), is operably cloned into a mutagenesis vector. The primers are designed to reflect the desired amino acid substitution. Other methods useful for the generation of variant Fc regions are known in the art (see, e.g., U.S. Pat. Nos. 5,624,821; 5,885,573; 5,677,425; 6,165,745; 6,277,375; 5,869,046; 6,121,022; 5,624,821; 5,648,260; 6,528,624; 6,194,551; 6,737,056; 6,821,505; 6,277,375; U.S. Patent Publication Nos. 2004/0002587 and PCT Publications WO 94/29351; WO 99/58572; WO 00/42072; WO 02/060919; WO 04/029207; WO 04/099249; WO 04/063351).

In some embodiments of the invention, the glycosylation patterns of the antibodies provided herein are modified to enhance ADCC and CDC effector function. See Shields R L et al., (2002) JBC. 277:26733; Shinkawa T et al., (2003) JBC. 278:3466 and Okazaki A et al., (2004) J. Mol. Biol., 336: 1239. In some embodiments, an Fc variant protein comprises one or more engineered glycoforms, i.e., a carbohydrate composition that is covalently attached to the molecule comprising an Fc region. Engineered glycoforms may be useful for a variety of purposes, including but not limited to enhancing or reducing effector function. Engineered glycoforms may be generated by any method known to one skilled in the art, for example by using engineered or variant expression strains, by co-expression with one or more enzymes, for example DI N-acetylglucosaminyltransferase III (GnTI11), by expressing a molecule comprising an Fc region in various organisms or cell lines from various organisms, or by modifying carbohydrate(s) after the molecule comprising Fc region has been expressed. Methods for generating engineered glycoforms are known in the art, and include but are not limited to those described in Umana et al, 1999, Nat. Biotechnol 17:176-180; Davies et al., 20017 Biotechnol Bioeng 74:288-294; Shields et al, 2002, J Biol Chem 277:26733-26740; Shinkawa et al., 2003, J Biol Chem 278:3466-3473) U.S. Pat. No. 6,602,684; U.S. Ser. No. 10/277,370; U.S. Ser. No. 10/113,929; PCT WO 00/61739A1; PCT WO 01/292246A1; PCT WO 02/311140A1; PCT WO 02/30954A1; Potillegent™ technology (Biowa, Inc. Princeton, N.J.); GlycoMAb™ glycosylation engineering technology (GLYCART biotechnology AG, Zurich, Switzerland). See, e.g., WO 00061739; EA01229125; US 20030115614; Okazaki et al., 2004, JMB, 336: 1239-49.

It is also known in the art that the glycosylation of the Fc region can be modified to increase or decrease effector function (see for examples, Umana et al, 1999, Nat. Biotechnol 17:176-180; Davies et al., 2001, Biotechnol Bioeng 74:288-294; Shields et al, 2002, J Biol Chem 277:26733-26740; Shinkawa et al., 2003, J Biol Chem 278:3466-3473) U.S. Pat. No. 6,602,684; U.S. Ser. No. 10/277,370; U.S. Ser. No. 10/113,929; PCT WO 00/61739A1; PCT WO 01/292246A1; PCT WO 02/311140A1; PCT WO 02/30954A1; Potillegent™ technology (Biowa, Inc. Princeton, N.J.); GlycoMAb™ glycosylation engineering technology (GLYCART biotechnology AG, Zurich, Switzerland). Accordingly, in one embodiment the Fc regions of the antibodies of the invention comprise altered glycosylation of amino acid residues. In another embodiment, the altered glycosylation of the amino acid residues results in lowered effector function. In another embodiment, the altered glycosylation of the amino acid residues results in increased effector function. In a specific embodiment, the Fc region has reduced fucosylation. In another embodiment, the Fc region is afucosylated (see for examples, U.S. Patent Application Publication No. 2005/0226867).

### BRIEF DESCRIPTION OF THE DRAWINGS

FIG. 1 is a bar chart showing the effect of inhibitory α5β1 antibodies on the attachment of endothelial cells to fibronectin, in the presence of 10 ug/ml L230. Antibody treatments are indicated on the X-axis, and compared to a no treatment control. IgG1 control, 3C5 and 5B11 were used at 10 μg/ml and 25 μg/ml as indicated. The mean cell adhesion as measured by cell count is indicated on the Y-axis, together with the standard deviation of the mean (error bars).

FIG. 2 is a bar chart showing the effect of inhibitory α5β1 antibodies on endothelial cell tube formation in an endothelial tube formation co-culture assay. Antibodies are indicated on the X-axis and concentrations from left to right in each group of bars are 5 μg/mL, 1 μg/mL, 0.2 μg/mL and 0.04 μg/mL. The degree of tube formation in terms of length (mm) and bifurcations is shown on the Y-axis. The values represented are the mean +/− the standard deviation. Vessel length (mm) is represented in black bars and bifurcations in grey bars.

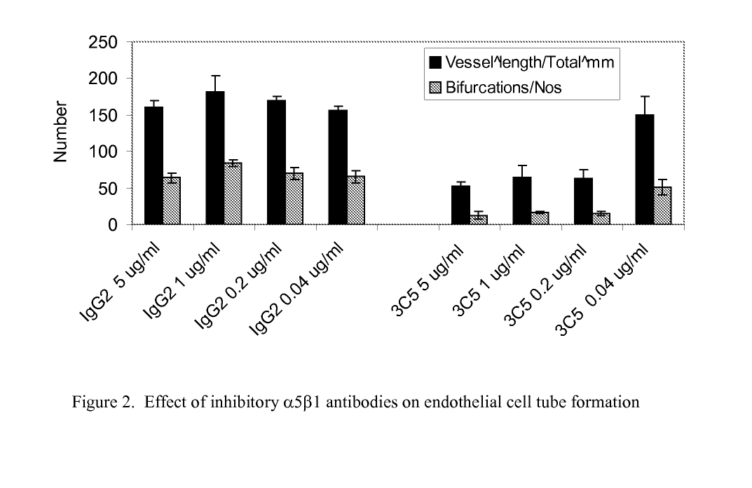

FIG. 3 is a bar chart showing the effect of inhibitory α5β1 antibodies on angiogenesis in vivo. Treatments are represented on the X-axis: Control vehicle twice weekly; 3C5 20 mg/kg twice weekly (diagonal stripped bars); 3C5 10 mg/kg twice weekly (checked bars); The Y axis shows mean vessel density +/− the standard error.

### DETAILED DESCRIPTION OF THE PREFERRED EMBODIMENT

Embodiments of the invention relate to targeted binding agents that bind to α5β1. In some embodiments, the targeted binding agents bind to α5β1 and inhibit the binding of fibronectin, fibrin, the adhesion molecule L1-CAM and growth factor receptors such as Tie-2 and Flt1 to α5β1. In one embodiment, the targeted binding agents are monoclonal antibodies, or binding fragments thereof. Such monoclonal antibodies may be referred to as anti-α5β1 antibodies herein.

Other embodiments of the invention include fully human anti-α5β1 antibodies, and antibody preparations that are therapeutically useful. In one embodiment, preparations of the anti-α5β1 antibody of the invention have desirable therapeutic properties, including strong binding affinity for α5β1 and the ability to inhibit α513′-induced cell activity in vitro and in vivo.

In addition, embodiments of the invention include methods of using these antibodies for treating diseases. Anti-α5β1 antibodies of the invention are useful for preventing α5β1-mediated tumourigenesis and tumour invasion of healthy tissue. In addition, α5β1 antibodies can be useful for treating diseases associated with angiogenesis such as ocular disease such as AMD, inflammatory disorders such as rheumatoid arthritis, and cardiovascular disease and sepsis as well as neoplastic diseases. Diseases that are treatable through this inhibition mechanism include, but are not limited to a neoplastic disease. Any disease that is characterized by any type of malignant tumour, including metastatic cancers, lymphatic tumours, and blood cancers, can also be treated by this inhibition mechanism. Exemplary cancers in humans include a bladder tumour, breast tumour, prostate tumour, basal cell carcinoma, biliary tract cancer, bladder cancer, bone cancer, brain and CNS cancer (e.g., glioma tumour), cervical cancer, choriocarcinoma, colon and rectum cancer, connective tissue cancer, cancer of the digestive system; endometrial cancer, esophageal cancer; eye cancer; cancer of the head and neck; gastric cancer; intra-epithelial neoplasm; kidney cancer; larynx cancer; leukemia; liver cancer; lung cancer (e.g. small cell and non-small cell); lymphoma including Hodgkin's and Non-Hodgkin's lymphoma; melanoma; myeloma, neuroblastoma, oral cavity cancer (e.g., lip, tongue, mouth, and pharynx); ovarian cancer; pancreatic cancer, retinoblastoma; rhabdomyosarcoma; rectal cancer, renal cancer, cancer of the respiratory system; sarcoma, skin cancer; stomach cancer, testicular cancer, thyroid cancer; uterine cancer, cancer of the urinary system, as well as other carcinomas and sarcomas. Malignant disorders commonly diagnosed in dogs, cats, and other pets include, but are not limited to, lymphosarcoma, osteosarcoma, mammary tumours, mastocytoma, brain tumour, melanoma, adenosquamous carcinoma, carcinoid lung tumour, bronchial gland tumour, bronchiolar adenocarcinoma, fibroma, myxochondroma, pulmonary sarcoma, neurosarcoma, osteoma, papilloma, retinoblastoma, Ewing's sarcoma, Wilm's tumour, Burkitt's lymphoma, microglioma, neuroblastoma, osteoclastoma, oral neoplasia, fibrosarcoma, osteosarcoma and rhabdomyosarcoma, genital squamous cell carcinoma, transmissible venereal tumour, testicular tumour, seminoma, Sertoli cell tumour, hemangiopericytoma, histiocytoma, chloroma (e.g., granulocytic sarcoma), corneal papilloma, corneal squamous cell carcinoma, hemangiosarcoma, pleural mesothelioma, basal cell tumour, thymoma, stomach tumour, adrenal gland carcinoma, oral papillomatosis, hemangioendothelioma and cystadenoma, follicular lymphoma, intestinal lymphosarcoma, fibrosarcoma and pulmonary squamous cell carcinoma. In rodents, such as a ferret, exemplary cancers include insulinoma, lymphoma, sarcoma, neuroma, pancreatic islet cell tumour, gastric MALT lymphoma and gastric adenocarcinoma. Neoplasias affecting agricultural livestock include leukemia, hemangiopericytoma and bovine ocular neoplasia (in cattle); preputial fibrosarcoma, ulcerative squamous cell carcinoma, preputial carcinoma, connective tissue neoplasia and mastocytoma (in horses); hepatocellular carcinoma (in swine); lymphoma and pulmonary adenomatosis (in sheep); pulmonary sarcoma, lymphoma, Rous sarcoma, reticulo-endotheliosis, fibrosarcoma, nephroblastoma, B-cell lymphoma and lymphoid leukosis (in avian species); retinoblastoma, hepatic neoplasia, lymphosarcoma (lymphoblastic lymphoma), plasmacytoid leukemia and swimbladder sarcoma (in fish), caseous lumphadenitis (CLA): chronic, infectious, contagious disease of sheep and goats caused by the bacterium Corynebacterium pseudotuberculosis, and contagious lung tumour of sheep caused by jaagsiekte.

Other embodiments of the invention include diagnostic assays for specifically determining the quantity of α5β1 in a biological sample. The assay kit can include a targeted binding agent or antibody as disclosed herein along with the necessary labels for detecting such antibodies. These diagnostic assays are useful to screen for cell adhesion, invasion, angiogenesis or proliferation-related diseases including, but not limited to, neoplastic diseases.

Another aspect of the invention is an antagonist of the biological activity of α5β1 wherein the antagonist binds to α5β1. In one embodiment, the antagonist is a targeted binding agent, such as an antibody. In one embodiment the antagonist is able to antagonize the biological activity of α5β1 in vitro and in vivo. The antagonist may be selected from an antibody described herein, for example, antibody 3C5 or 5B11.

In one embodiment the antagonist of the biological activity of α5β1 binds to α5β1 thereby inhibiting cell adhesion and/or invasion and/or angiogenesis and/or proliferation. The mechanism of action of this inhibition may include binding of the antagonist to α5β1 and inhibiting the binding of a native α5β1-specific ligand, such as, for example fibronectin, to α5β1. Without wishing to be bound by any particular theoretical considerations, mechanisms by which antagonism of the biological activity of α5β1 can be achieved include, but are not limited to, inhibition of binding of fibronectin to α5β1, and/or inhibition of α5β1-fibronectin mediated signaling activity.

One embodiment is a hybridoma that produces the targeted binding agent as described hereinabove. In one embodiment is a hybridoma that produces the light chain and/or the heavy chain of the antibodies as described hereinabove. In one embodiment the hybridoma produces the light chain and/or the heavy chain of a fully human monoclonal antibody. In another embodiment the hybridoma produces the light chain and/or the heavy chain of fully human monoclonal antibody 3C5 or 5B11. Alternatively the hybridoma may produce an antibody which binds to the same epitope or epitopes as fully human monoclonal antibody 3C5 or 5B11.

Another embodiment is a nucleic acid molecule encoding the targeted binding agent as described hereinabove. In one embodiment is a nucleic acid molecule encoding the light chain or the heavy chain of an antibody as described hereinabove. In one embodiment the nucleic acid molecule encodes the light chain or the heavy chain of a fully human monoclonal antibody. Still another embodiment is a nucleic acid molecule encoding the light chain or the heavy chain of a fully human monoclonal antibody selected from antibodies 3C5 or 5B11.

Another embodiment of the invention is a vector comprising a nucleic acid molecule or molecules as described hereinabove, wherein the vector encodes a targeted binding agent as defined hereinabove. In one embodiment of the invention is a vector comprising a nucleic acid molecule or molecules as described hereinabove, wherein the vector encodes a light chain and/or a heavy chain of an antibody as defined hereinabove.

Yet another embodiment of the invention is a host cell comprising a vector as described hereinabove. Alternatively the host cell may comprise more than one vector.

In addition, one embodiment of the invention is a method of producing a targeted binding agent of the invention by culturing host cells under conditions wherein a nucleic acid molecule is expressed to produce the targeted binding agent, followed by recovery of the targeted binding agent. In one embodiment of the invention is a method of producing an antibody of the invention by culturing host cells under conditions wherein a nucleic acid molecule is expressed to produce the antibody, followed by recovery of the antibody.

In one embodiment the invention includes a method of making an targeted binding agent by transfecting at least one host cell with at least one nucleic acid molecule encoding the targeted binding agent as described hereinabove, expressing the nucleic acid molecule in the host cell and isolating the targeted binding agent. In one embodiment the invention includes a method of making an antibody by transfecting at least one host cell with at least one nucleic acid molecule encoding the antibody as described hereinabove, expressing the nucleic acid molecule in the host cell and isolating the antibody.

According to another aspect, the invention includes a method of antagonising the biological activity of α5β1 by administering an antagonist as described herein. The method may include selecting an animal in need of treatment for disease-related cell adhesion and/or invasion and/or angiogenesis and/or proliferation, and administering to the animal a therapeutically effective dose of an antagonist of the biological activity of α5β1.

Another aspect of the invention includes a method of antagonising the biological activity of α5β1 by administering a targeted binding agent as described hereinabove. The method may include selecting an animal in need of treatment for disease-related cell adhesion and/or invasion and/or angiogenesis and/or proliferation, and administering to the animal a therapeutically effective dose of a targeted binding agent which antagonises the biological activity of α5β1.

Another aspect of the invention includes a method of antagonising the biological activity of α5β1 by administering an antibody as described hereinabove. The method may include selecting an animal in need of treatment for disease-related cell adhesion and/or invasion and/or angiogenesis and/or proliferation, and administering to the animal a therapeutically effective dose of an antibody which antagonises the biological activity of α5β1.

According to another aspect there is provided a method of treating disease-related cell adhesion and/or invasion and/or angiogenesis and/or proliferation in an animal by administering a therapeutically effective amount of an antagonist of the biological activity of α5β1. The method may include selecting an animal in need of treatment for disease-related cell adhesion and/or invasion and/or angiogenesis and/or proliferation, and administering to the animal a therapeutically effective dose of an antagonist of the biological activity of α5β1.

According to another aspect there is provided a method of treating disease-related cell adhesion and/or invasion and/or angiogenesis and/or proliferation in an animal by administering a therapeutically effective amount of a targeted binding agent which antagonizes the biological activity of α5β1. The method may include selecting an animal in need of treatment for disease-related cell adhesion and/or invasion and/or angiogenesis and/or proliferation, and administering to the animal a therapeutically effective dose of a targeted binding agent which antagonises the biological activity of α5β1. The targeted binding agent can be administered alone, or can be administered in combination with additional antibodies or chemotherapeutic drugs or radiation therapy.

According to another aspect there is provided a method of treating disease-related cell adhesion and/or invasion and/or angiogenesis and/or proliferation in an animal by administering a therapeutically effective amount of an antibody which antagonizes the biological activity of α5β1. The method may include selecting an animal in need of treatment for disease-related cell adhesion and/or invasion and/or angiogenesis and/or proliferation, and administering to the animal a therapeutically effective dose of an antibody which antagonises the biological activity of α5β1. The antibody can be administered alone, or can be administered in combination with additional antibodies or chemotherapeutic drugs or radiation therapy.

According to another aspect there is provided a method of treating cancer in an animal by administering a therapeutically effective amount of an antagonist of the biological activity of α5β1. The method may include selecting an animal in need of treatment for cancer, and administering to the animal a therapeutically effective dose of an antagonist which antagonises the biological activity of α5β1. The antagonist can be administered alone, or can be administered in combination with additional antibodies or chemotherapeutic drugs or radiation therapy.

According to another aspect there is provided a method of treating cancer in an animal by administering a therapeutically effective amount of a targeted binding agent which antagonizes the biological activity of α5β1. The method may include selecting an animal in need of treatment for cancer, and administering to the animal a therapeutically effective dose of a targeted binding agent which antagonises the biological activity of α5β1. The targeted binding agent can be administered alone, or can be administered in combination with additional antibodies or chemotherapeutic drugs or radiation therapy.

According to another aspect there is provided a method of treating cancer in an animal by administering a therapeutically effective amount of an antibody which antagonizes the biological activity of α5β1. The method may include selecting an animal in need of treatment for cancer, and administering to the animal a therapeutically effective dose of an antibody which antagonises the biological activity of α5β1. The antibody can be administered alone, or can be administered in combination with additional antibodies or chemotherapeutic drugs or radiation therapy.

According to another aspect there is provided a method of reducing or inhibiting tumour cell proliferation, adhesion, invasion and/or angiogenesis, in an animal by administering a therapeutically effective amount of an antibody which antagonizes the biological activity of α5β1. The method may include selecting an animal in need of a reduction or inhibition of proliferation, cell adhesion, invasion and/or angiogenesis, and administering to the animal a therapeutically effective dose of an antibody which antagonises the biological activity of α5β1. The antibody can be administered alone, or can be administered in combination with additional antibodies or chemotherapeutic drugs or radiation therapy.

According to another aspect there is provided a method of reducing tumour growth and/or metastasis, in an animal by administering a therapeutically effective amount of an antibody which antagonizes the biological activity of α5β1. The method may include selecting an animal in need of a reduction of tumour growth and/or metastasis, and administering to the animal a therapeutically effective dose of an antibody which antagonises the biological activity of α5β1. The antibody can be administered alone, or can be administered in combination with additional antibodies or chemotherapeutic drugs or radiation therapy.

According to another aspect of the invention there is provided the use of an antagonist of the biological activity of α5β1 for the manufacture of a medicament for the treatment of disease-related cell adhesion and/or invasion and/or angiogenesis and/or proliferation. In one embodiment the antagonist of the biological activity of α5β1 is a targeted binding agent of the invention. In one embodiment the antagonist of the biological activity of α5β1 is an antibody of the invention.

According to another aspect of the invention there is provided an antagonist of the biological activity of α5β1 for use as a medicament for the treatment of disease-related cell adhesion and/or invasion and/or angiogenesis and/or proliferation. In one embodiment the antagonist of the biological activity of α5β1 is a targeted binding agent of the invention. In one embodiment the antagonist of the biological activity of α5β1 is an antibody of the invention.

According to another aspect of the invention there is provided the use of a targeted binding agent or an antibody which antagonizes the biological activity of α5β1 for the manufacture of a medicament for the treatment of disease-related cell adhesion and/or invasion and/or angiogenesis and/or proliferation.

According to another aspect of the invention there is provided a targeted binding agent or an antibody which antagonizes the biological activity of α5β1 for use as a medicament for the treatment of disease-related cell adhesion and/or invasion and/or angiogenesis and/or proliferation.

According to another aspect of the invention there is provided the use of a targeted binding agent or an antibody which antagonizes the biological activity of α5β1 for the manufacture of a medicament for the treatment of disease-related cell adhesion and/or invasion and/or angiogenesis and/or proliferation.

According to another aspect of the invention there is provided an antibody which antagonizes the biological activity of α5β1 for use as a medicament for the treatment of disease-related cell adhesion and/or invasion and/or angiogenesis and/or proliferation.

According to another aspect of the invention there is provided the use of an antagonist of the biological activity of α5β1 for the manufacture of a medicament for the treatment of cancer in a mammal. In one embodiment the antagonist of the biological activity of α5β1 is a targeted binding agent of the invention. In one embodiment the antagonist of the biological activity of α5β1 is an antibody of the invention.

According to another aspect of the invention there is provided an antagonist of the biological activity of α5β1 for use as a medicament for the treatment of cancer in a mammal. In one embodiment the antagonist of the biological activity of α5β1 is a targeted binding agent of the invention. In one embodiment the antagonist of the biological activity of α5β1 is an antibody of the invention.

According to another aspect of the invention there is provided the use of a targeted binding agent which antagonizes the biological activity of α5β1 for the manufacture of a medicament for the treatment of cancer in a mammal

According to another aspect of the invention there is provided a targeted binding agent which antagonizes the biological activity of α5β1 for use as a medicament for the treatment of cancer in a mammal

According to another aspect of the invention there is provided the use of an antibody which antagonizes the biological activity of α5β1 for the manufacture of a medicament for the treatment of cancer in a mammal.

According to another aspect of the invention there is provided an antibody which antagonizes the biological activity of α5β1 for use as a medicament for the treatment of cancer in a mammal

According to another aspect there is provided the use of a targeted binding agent or an antibody which antagonizes the biological activity of α5β1 for the manufacture of a medicament for the reduction or inhibition proliferation, cell adhesion, invasion and/or angiogenesis in an animal.

According to another aspect there is provided a targeted binding agent or an antibody which antagonizes the biological activity of α5β1 for use as a medicament for the reduction or inhibition proliferation, cell adhesion, invasion and/or angiogenesis in an animal.

According to another aspect there is provided the use of a targeted binding agent or an antibody which antagonizes the biological activity of α5β1 for the manufacture of a medicament for reducing tumour growth and/or metastasis, in an animal.

According to another aspect there is provided a targeted binding agent or an antibody which antagonizes the biological activity of α5β1 for use as a medicament for reducing tumour growth and/or metastasis, in an animal.

In one embodiment the present invention is particularly suitable for use in antagonizing α5β1, in patients with a tumour which is dependent alone, or in part on α5β1. According to another aspect of the invention there is provided a pharmaceutical composition comprising an antagonist of the biological activity of α5β1, and a pharmaceutically acceptable carrier. In one embodiment the antagonist comprises an antibody. According to another aspect of the invention there is provided a pharmaceutical composition comprising an antagonist of the biological activity of α5β1, and a pharmaceutically acceptable carrier. In one embodiment the antagonist comprises an antibody.

In some embodiments, following administration of the antibody that specifically binds to α5β1, a clearing agent is administered, to remove excess circulating antibody from the blood.

Anti-α5β1 antibodies are useful in the detection of α5β1 in patient samples and accordingly are useful as diagnostics for disease states as described herein. In addition, based on their ability to significantly inhibit α5β1-mediated signaling activity (as demonstrated in the Examples below), anti-α5β1 antibodies have therapeutic effects in treating symptoms and conditions resulting from α5β1 expression. In specific embodiments, the antibodies and methods herein relate to the treatment of symptoms resulting from and induced cell adhesion, invasion, angiogenesis, proliferation and/or intracellular signaling. Further embodiments involve using the antibodies and methods described herein to treat cell adhesion, invasion, angiogenesis and/or proliferation-related diseases including neoplastic diseases, such as, melanoma, small cell lung cancer, non-small cell lung cancer, glioma, hepatocellular (liver) carcinoma, thyroid tumour, gastric (stomach) cancer, prostate cancer, breast cancer, ovarian cancer, bladder cancer, lung cancer, glioblastoma, endometrial cancer, kidney cancer, colon cancer, and pancreatic cancer. The antibodies may also be useful in treating cell adhesion and/or invasion in arthritis, atherosclerosis and diseases involving angiogenesis.

Another embodiment of the invention includes an assay kit for detecting α5β1 in mammalian tissues, cells, or body fluids to screen for cell adhesion-, invasion-, angiogenesis- or proliferation related diseases. The kit includes a targeted binding agent that binds to α5β1 and a means for indicating the reaction of the targeted binding agent with α5β1, if present. In one embodiment, the targeted binding agent that binds α5β1 is labeled. In another embodiment the targeted binding agent is an unlabeled and the kit further includes a means for detecting the targeted binding agent. Preferably the targeted binding agent is labeled with a marker selected from the group consisting of a fluorochrome, an enzyme, a radionuclide and a radio-opaque material.

Another embodiment of the invention includes an assay kit for detecting α5β1 in mammalian tissues, cells, or body fluids to screen for cell adhesion-, invasion-, angiogenesis or proliferation-related diseases. The kit includes an antibody that binds to α5β1 and a means for indicating the reaction of the antibody with α5β1, if present. The antibody may be a monoclonal antibody. In one embodiment, the antibody that binds α5β1 is labeled. In another embodiment the antibody is an unlabeled primary antibody and the kit further includes a means for detecting the primary antibody. In one embodiment, the means includes a labeled second antibody that is an anti-immunoglobulin. Preferably the antibody is labeled with a marker selected from the group consisting of a fluorochrome, an enzyme, a radionuclide and a radio-opaque material.

Further embodiments, features, and the like regarding the antibodies as disclosed herein are provided in additional detail below.

### Sequence Listing

Embodiments of the invention include the specific antibodies listed below in Table 1. This table reports the identification number of each anti-α5β1 antibody, along with the SEQ ID number of the variable domain of the corresponding heavy chain and light chain genes and polypeptides, respectively. Each antibody has been given an identification number.

TABLE 1

| MAb ID | | SEQ ID |
|---|---|---|
| No.: | Sequence | NO: |
| 3G11.1A6 | Nucleotide sequence encoding the variable region of the heavy chain | 1 |
| | Amino acid sequence encoding the variable region of the heavy chain | 2 |
| | Nucleotide sequence encoding the variable region of the light chain | 3 |
| | Amino acid sequence encoding the variable region of the light chain | 4 |
| 2E10.1B9 | Nucleotide sequence encoding the variable region of the heavy chain | 5 |
| | Amino acid sequence encoding the variable region of the heavy chain | 6 |
| | Nucleotide sequence encoding the variable region of the light chain | 7 |
| | Amino acid sequence encoding the variable region of the light chain | 8 |
| 2A9.1A1 | Nucleotide sequence encoding the variable region of the heavy chain | 9 |
| | Amino acid sequence encoding the variable region of the heavy chain | 10 |
| | Nucleotide sequence encoding the variable region of the light chain | 11 |
| | Amino acid sequence encoding the variable region of the light chain | 12 |
| 2C5.2B12 | Nucleotide sequence encoding the variable region of the heavy chain | 13 |
| | Amino acid sequence encoding the variable region of the heavy chain | 14 |
| | Nucleotide sequence encoding the variable region of the light chain | 15 |
| | Amino acid sequence encoding the variable region of the light chain | 16 |
| 3C2.2A8 | Nucleotide sequence encoding the variable region of the heavy chain | 17 |
| | Amino acid sequence encoding the variable region of the heavy chain | 18 |
| | Nucleotide sequence encoding the variable region of the light chain | 19 |
| | Amino acid sequence encoding the variable region of the light chain | 20 |
| 3C5 | Nucleotide sequence encoding the variable region of the heavy chain | 21 |
| | Amino acid sequence encoding the variable region of the heavy chain | 22 |
| | Nucleotide sequence encoding the variable region of the light chain | 23 |
| | Amino acid sequence encoding the variable region of the light chain | 24 |
| | Amino acid sequence encoding the CDR1 region of the heavy chain | 25 |
| | Amino acid sequence encoding the CDR2 region of the heavy chain | 26 |
| | Amino acid sequence encoding the CDR3 region of the heavy chain | 27 |
| | Amino acid sequence encoding the CDR1 region of the light chain | 28 |
| | Amino acid sequence encoding the CDR2 region of the light chain | 29 |
| | Amino acid sequence encoding the CDR3 region of the light chain | 30 |
| 8A6.1A3 | Nucleotide sequence encoding the variable region of the heavy chain | 31 |
| | Amino acid sequence encoding the variable region of the heavy chain | 32 |
| | Nucleotide sequence encoding the variable region of the light chain | 33 |
| | Amino acid sequence encoding the variable region of the light chain | 34 |
| 9E2.3A8 | Nucleotide sequence encoding the variable region of the heavy chain | 35 |
| | Amino acid sequence encoding the variable region of the heavy chain | 36 |
| | Nucleotide sequence encoding the variable region of the light chain | 37 |
| | Amino acid sequence encoding the variable region of the light chain | 38 |
| 2F5.1A4 | Nucleotide sequence encoding the variable region of the heavy chain | 39 |
| | Amino acid sequence encoding the variable region of the heavy chain | 40 |
| | Nucleotide sequence encoding the variable region of the light chain | 41 |
| | Amino acid sequence encoding the variable region of the light chain | 42 |
| 7B2.3B10 | Nucleotide sequence encoding the variable region of the heavy chain | 43 |
| | Amino acid sequence encoding the variable region of the heavy chain | 44 |
| | Nucleotide sequence encoding the variable region of the light chain | 45 |
| | Amino acid sequence encoding the variable region of the light chain | 46 |
| 5B11 | Nucleotide sequence encoding the variable region of the heavy chain | 47 |
| | Amino acid sequence encoding the variable region of the heavy chain | 48 |
| | Nucleotide sequence encoding the variable region of the light chain | 49 |
| | Amino acid sequence encoding the variable region of the light chain | 50 |
| | Amino acid sequence encoding the CDR1 region of the heavy chain | 51 |
| | Amino acid sequence encoding the CDR2 region of the heavy chain | 52 |
| | Amino acid sequence encoding the CDR3 region of the heavy chain | 53 |
| | Amino acid sequence encoding the CDR1 region of the light chain | 54 |
| | Amino acid sequence encoding the CDR2 region of the light chain | 55 |
| | Amino acid sequence encoding the CDR3 region of the light chain | 56 |
| | Germline sequence VH4-31, D2-2, JH6B | 57 |
| | Germline sequence VH1-2, D6-19, JH4B | 58 |
| | Germline sequence VH1-2, D6-19, JH5B | 59 |
| | Germline sequence VH3-33, D4-23, JH4B | 60 |
| | Germline sequence VH3-33, D6-13, JH4B | 61 |
| | Germline sequence A3, JK3 | 62 |
| | Germline sequence B3, JK1 | 63 |
| | Germline sequence O2, JK1 | 64 |
| | Germline sequence L1, JK4 | 65 |

### DEFINITIONS

Unless otherwise defined, scientific and technical terms used herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art.

Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (3rd ed., Cold Spring Harbor Laboratory Press, Cold Spring Harbor, N.Y. (2001)), which is incorporated herein by reference. The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

An antagonist or inhibitor may be a polypeptide, nucleic acid, carbohydrate, lipid, small molecular weight compound, an oligonucleotide, an oligopeptide, RNA interference (RNAi), antisense, a recombinant protein, an antibody, or fragments thereof or conjugates or fusion proteins thereof. For a review of RNAi see Milhavet O, Gary D S, Mattson M P. (Pharmacol Rev. 2003 December; 55(4):629-48. Review) and antisense (see Opalinska J B, Gewirtz A M. (Sci STKE. Oct. 28; 2003 (206):pe47.)

Disease-related cell adhesion and/or invasion and/or angiogenesis and/or proliferation may be any abnormal, undesirable or pathological cell adhesion and/or invasion and/or angiogenesis and/or proliferation, for example tumour-related cell adhesion and/or invasion and/or angiogenesis and/or proliferation. Cell adhesion- and/or invasion and/or angiogenesis- and/or proliferation-related diseases include, but are not limited to, non-solid tumours such as leukemia, multiple myeloma or lymphoma, and also solid tumours such as melanoma, small cell lung cancer, non-small cell lung cancer, glioma, hepatocellular (liver) carcinoma, glioblastoma, carcinoma of the thyroid, bile duct, bone, gastric, brain/CNS, head and neck, hepatic system, stomach, prostate, breast, renal, testicle, ovary, skin, cervix, lung, muscle, neuron, esophageal, bladder, lung, uterus, vulva, endometrium, kidney, colorectum, pancreas, pleural/peritoneal membranes, salivary gland, and epidermous.

A compound refers to any small molecular weight compound with a molecular weight of less than about 2000 Daltons.

The term “α5β1” refers to the molecule that is α5β1 protein.

The term “allotype” is used with respect to antigenic determinants specified by allelic forms of antibody genes. Allotypes represent slight differences in the amino acid sequences of heavy or light chains of different individuals and are sequence differences between alleles of a subclass whereby an antisera recognize only the allelic differences. The most important types are Gm (heavy chain) and Km (light chain). Gm polymorphism is determined by IGHG1, IGHG2 and IGHG3 genes which have alleles encoding allotypic antigenic determinants referred to as G1m, G2m, and G3 allotypes for markers of the IgG1, IgG2 and IgG2 molecules. At present, 18 Gm allotypes are known: G1m (1,2,3,17) or G1m (a, x, f, z), G2m (23) or G2m (n), G3m (5, 6, 10, 11, 13, 14, 15, 16, 21, 24, 26, 27, 28) pr G3m (b1, c3, b5, b0, b3, b4, s, t, g, 1, c5, u, v, g5) (Lefranc, et al., The human IgG subclasses: molecular analysis of structure, function and regulation. Pergamon, Oxford, pp. 43-78 (1990); Lefranc, G. et al., 1979, Hum. Genet.: 50, 199-21 1, both incorporated entirely by reference).

Allelic forms of human immunoglobulins have been well-characterized (WHO Review of the notation for the allotypic and related markers of human immunoglobulins J Immunogen 1976, 3: 357-362; WHO Review of the notation for the allotypic and related markers of human immunoglobulins. 1976, Eur. J. Immunol. 6, 599-601; E. van Loghem, 1986, Allotypic markers, Monogr Allergy 19:40-51, all incorporated entirely by reference). Additionally, other polymorphisms have been characterized (Kim et al., 2001, J. Mol. Evol. 54:1-9, incorporated entirely by reference).

The terms “neutralizing” or “inhibits” when referring to a targeted binding agent, such as an antibody, relates to the ability of an antibody to eliminate, reduce, or significantly reduce, the activity of a target antigen. Accordingly, a “neutralizing” anti-α5β1 antibody of the invention is capable of eliminating or significantly reducing the activity of α5β1. A neutralizing α5β1 antibody may, for example, act by blocking the binding of a native α5β1-specific ligand, such as, for example, fibronectin, to α5β1. By blocking this binding, α5β1 signal-mediated activity is significantly, or completely, eliminated. Ideally, a neutralizing antibody against α5β1 inhibits cell adhesion and/or invasion and/or angiogenesis and/or proliferation.

An “antagonist of the biological activity of α5β1” is capable of eliminating, reducing or significantly reducing the activity of α5β1. An “antagonist of the biological activity of α5β1” is capable of eliminating, reducing or significantly reducing α5β1 signaling. An “antagonist of the biological activity of α5β1” may eliminate or significantly reduce cell adhesion and/or invasion and/or angiogenesis and/or proliferation.

“Reducing α5β1 signaling” encompasses a reduction of α5β1 signaling by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% in comparison with the level of signaling in the absence of a targeted binding agent, antibody or antagonist of the invention.

The term “polypeptide” is used herein as a generic term to refer to native protein, fragments, or analogs of a polypeptide sequence. Hence, native protein, fragments, and analogs are species of the polypeptide genus. Preferred polypeptides in accordance with the invention comprise the human heavy chain immunoglobulin molecules and the human kappa light chain immunoglobulin molecules, as well as antibody molecules formed by combinations comprising the heavy chain immunoglobulin molecules with light chain immunoglobulin molecules, such as the kappa or lambda light chain immunoglobulin molecules, and vice versa, as well as fragments and analogs thereof. Preferred polypeptides in accordance with the invention may also comprise solely the human heavy chain immunoglobulin molecules or fragments thereof.

The terms “native” or “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally-occurring.

The term “operably linked” as used herein refers to positions of components so described that are in a relationship permitting them to function in their intended manner. For example, a control sequence “operably linked” to a coding sequence is connected in such a way that expression of the coding sequence is achieved under conditions compatible with the control sequences.

The term “polynucleotide” as referred to herein means a polymeric form of nucleotides of at least 10 bases in length, either ribonucleotides or deoxynucleotides or a modified form of either type of nucleotide, or RNA-DNA hetero-duplexes. The term includes single and double stranded forms of DNA.

The term “oligonucleotide” referred to herein includes naturally occurring, and modified nucleotides linked together by naturally occurring, and non-naturally occurring linkages. Oligonucleotides are a polynucleotide subset generally comprising a length of 200 bases or fewer. Preferably, oligonucleotides are 10 to 60 bases in length and most preferably 12, 13, 14, 15, 16, 17, 18, 19, or 20 to 40 bases in length. Oligonucleotides are usually single stranded, e.g. for probes; although oligonucleotides may be double stranded, e.g. for use in the construction of a gene mutant. Oligonucleotides can be either sense or antisense oligonucleotides.

The term “naturally occurring nucleotides” referred to herein includes deoxyribonucleotides and ribonucleotides. The term “modified nucleotides” referred to herein includes nucleotides with modified or substituted sugar groups and the like. The term “oligonucleotide linkages” referred to herein includes oligonucleotides linkages such as phosphorothioate, phosphorodithioate, phosphoroselenoate, phosphorodiselenoate, phosphoroanilothioate, phosphoraniladate, phosphoroamidate, and the like. See e.g., LaPlanche et al. Nucl. Acids Res. 14:9081 (1986); Stec et al. J. Am. Chem. Soc. 106:6077 (1984); Stein et al. Nucl. Acids Res. 16:3209 (1988); Zon et al. Anti-Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990), the disclosures of which are hereby incorporated by reference. An oligonucleotide can include a label for detection, if desired.

The term “selectively hybridise” referred to herein means to detectably and specifically bind. Polynucleotides, oligonucleotides and fragments thereof selectively hybridise to nucleic acid strands under hybridisation and wash conditions that minimise appreciable amounts of detectable binding to nonspecific nucleic acids. High stringency conditions can be used to achieve selective hybridisation conditions as known in the art and discussed herein. Generally, the nucleic acid sequence homology between the polynucleotides, oligonucleotides, or antibody fragments and a nucleic acid sequence of interest will be at least 80%, and more typically with preferably increasing homologies of at least 85%, 90%, 95%, 99%, and 100%.

Stringent hybridization conditions include, but are not limited to, hybridization to filter-bound DNA in 6× sodium chloride/sodium citrate (SSC) (0.9 M NaCl/90 mM NaCitrate, pH 7.0) at about 45° C. followed by one or more washes in 0.2×SSC/0.1% SDS at about 50-65° C., highly stringent conditions such as hybridization to filter-bound DNA in 6×SSC at about 45° C. followed by one or more washes in 0.1×SSC/0.2% SDS at about 60° C., or any other stringent hybridization conditions known to those skilled in the art (see, for example, Ausubel, F. M. et al., eds. 1989 Current Protocols in Molecular Biology, vol. 1, Green Publishing Associates, Inc. and John Wiley and Sons, Inc., NY at pages 6.3.1 to 6.3.6 and 2.10.3). Two amino acid sequences are “homologous” if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least about 30 amino acids in length) are homologous, as this term is used herein, if they have an alignment score of more than 5 (in standard deviation units) using the program ALIGN with the mutation data matrix and a gap penalty of 6 or greater. See Dayhoff, M. O., in Atlas of Protein Sequence and Structure, pp. 101-110 (Volume 5, National Biomedical Research Foundation (1972)) and Supplement 2 to this volume, pp. 1-10. The two sequences or parts thereof are more preferably homologous if their amino acids are greater than or equal to 50% identical when optimally aligned using the ALIGN program. It should be appreciated that there can be differing regions of homology within two orthologous sequences. For example, the functional sites of mouse and human orthologues may have a higher degree of homology than non-functional regions.

The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence.

In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U, or I) or amino acid residue occurs in both sequences to yield the number of matched positions, dividing the number of matched positions by the total number of positions in the comparison window (i.e., the window size), and multiplying the result by 100 to yield the percentage of sequence identity. The terms “substantial identity” as used herein denotes a characteristic of a polynucleotide or amino acid sequence, wherein the polynucleotide or amino acid comprises a sequence that has at least 85 percent sequence identity, preferably at least 90 to 95 percent sequence identity, more preferably at least 99 percent sequence identity, as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which may include deletions or additions which total 20 percent or less of the reference sequence over the comparison window. The reference sequence may be a subset of a larger sequence.

As used herein, the twenty conventional amino acids and their abbreviations follow conventional usage. See Immunology—A Synthesis (2^{nd} Edition, E. S. Golub and D. R. Gren, Eds., Sinauer Associates, Sunderland, Mass. (1991)), which is incorporated herein by reference. Stereoisomers (e.g., D-amino acids) of the twenty conventional amino acids, unnatural amino acids such as α-, α-disubstituted amino acids, N-alkyl amino acids, lactic acid, and other unconventional amino acids may also be suitable components for polypeptides of the present invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

Similarly, unless specified otherwise, the left-hand end of single-stranded polynucleotide sequences is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA and which are 5′ to the 5′ end of the RNA transcript are referred to as “upstream sequences”; sequence regions on the DNA strand having the same sequence as the RNA and which are 3′ to the 3′ end of the RNA transcript are referred to as “downstream sequences”.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity, and most preferably at least 99 percent sequence identity. Preferably, residue positions that are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, glutamic-aspartic, and asparagine-glutamine.

As discussed herein, minor variations in the amino acid sequences of antibodies or immunoglobulin molecules are contemplated as being encompassed by the present invention, providing that the variations in the amino acid sequence maintain at least 75%, more preferably at least 80%, 90%, 95%, and most preferably 99% sequence identity to the antibodies or immunoglobulin molecules described herein. In particular, conservative amino acid replacements are contemplated. Conservative replacements are those that take place within a family of amino acids that have related side chains. Genetically encoded amino acids are generally divided into families: (1) acidic=aspartate, glutamate; (2) basic=lysine, arginine, histidine; (3) non-polar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are an aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding function or properties of the resulting molecule, especially if the replacement does not involve an amino acid within a framework site. Whether an amino acid change results in a functional peptide can readily be determined by assaying the specific activity of the polypeptide derivative. Assays are described in detail herein. Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). Thus, the foregoing examples demonstrate that those of skill in the art can recognize sequence motifs and structural conformations that may be used to define structural and functional domains in accordance with the antibodies described herein.

Glutaminyl and asparaginyl residues are frequently deamidated to the corresponding glutamyl and aspartyl residues, respectively. These residues are deamidated under neutral or basic conditions. The deamidated form of these residues falls within the scope of this invention.

In general, cysteine residues in proteins are either engaged in cysteine-cysteine disulfide bonds or sterically protected from the disulfide bond formation when they are a part of folded protein region. Disulfide bond formation in proteins is a complex process, which is determined by the redox potential of the environment and specialized thiol-disulfide exchanging enzymes (Creighton, Methods Enzymol. 107, 305-329, 1984; Houee-Levin, Methods Enzymol. 353, 35-44, 2002). When a cysteine residue does not have a pair in protein structure and is not sterically protected by folding, it can form a disulfide bond with a free cysteine from solution in a process known as disulfide shuffling. In another process known as disulfide scrambling, free cysteines may also interfere with naturally occurring disulfide bonds (such as those present in antibody structures) and lead to low binding, low biological activity and/or low stability.

Preferred amino acid substitutions are those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. Analogs can include various mutations of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W.H. Freeman and Company, New York (1984)); Introduction to Protein Structure (C. Branden and J. Tooze, eds., Garland Publishing, New York, N.Y. (1991)); and Thornton et al. Nature 354:105 (1991), which are each incorporated herein by reference.

Alteration may comprise replacing one or more amino acid residue(s) with a non-naturally occurring or non-standard amino acid, modifying one or more amino acid residue into a non-naturally occurring or non-standard form, or inserting one or more non-naturally occurring or non-standard amino acid into the sequence. Naturally occurring amino acids include the 20 “standard” L-amino acids identified as G, A, V, L, I, M, P, F, W, S, T, N, Q, Y, C, K, R, H, D, E by their standard single-letter codes. Non-standard amino acids include any other residue that may be incorporated into a polypeptide backbone or result from modification of an existing amino acid residue. Non-standard amino acids may be naturally occurring or non-naturally occurring.

Additionally, such methods may be used to make amino acid substitutions or deletions of one or more variable region cysteine residues participating in an intrachain disulfide bond to generate antibody molecules lacking one or more intrachain disulfide bonds.

The term “CDR region” or “CDR” is intended to indicate the hypervariable regions of the heavy and light chains of an antibody which confer antigen-binding specificity to the antibody. CDRs may be defined according to the Kabat system (Kabat, E. A. et al. (1991) Sequences of Proteins of Immunological Interest, 5th Edition. US Department of Health and Human Services, Public Service, NIH, Washington), and later editions. An antibody typically contains 3 heavy chain CDRs and 3 light chain CDRs. The term CDR or CDRs is used here in order to indicate, according to the case, one of these regions or several, or even the whole, of these regions which contain the majority of the amino acid residues responsible for the binding by affinity of the antibody for the antigen or the epitope which it recognises.

The third CDR of the heavy chain (HCDR3) has a greater size variability (greater diversity essentially due to the mechanisms of arrangement of the genes which give rise to it). It may be as short as 2 amino acids although the longest size known is 26. CDR length may also vary according to the length that can be accommodated by the particular underlying framework. Functionally, HCDR3 plays a role in part in the determination of the specificity of the antibody (Segal et al., PNAS, 71:4298-4302, 1974, Amit et al., Science, 233:747-753, 1986, Chothia et al., 10J. Mol. Biol., 196:901-917, 1987, Chothia et al., Nature, 342:877-883, 1989, Caton et al., J. Immunol., 144:1965-1968, 1990, Sharon et al., PNAS, 87:4814-4817, 1990, Sharon et al., J. Immunol., 144:4863-4869, 1990, Kabat et al., J. Immunol., 147:1709-1719, 1991).

The term a “set of CDRs” referred to herein comprises CDR1, CDR2 and CDR3. Thus, a set of HCDRs refers to HCDR1, HCDR2 and HCDR3, and a set of LCDRs refers to LCDR1, LCDR2 and LCDR3.

Variants of the VH and VL domains and CDRs of the present invention, including those for which amino acid sequences are set out herein, and which can be employed in targeting agents and antibodies for α5β1 can be obtained by means of methods of sequence alteration or mutation and screening for antigen targeting with desired characteristics. Examples of desired characteristics include but are not limited to: increased binding affinity for antigen relative to known antibodies which are specific for the antigen; increased neutralisation of an antigen activity relative to known antibodies which are specific for the antigen if the activity is known; specified competitive ability with a known antibody or ligand to the antigen at a specific molar ratio; ability to immunoprecipitate ligand-receptor complex; ability to bind to a specified epitope; linear epitope, e.g. peptide sequence identified using peptide-binding scan, e.g. using peptides screened in linear and/or constrained conformation; conformational epitope, formed by non-continuous residues; ability to modulate a new biological activity of α5β1, or downstream molecule; ability to bind and/or neutralise α5β1 and/or for any other desired property.

The techniques required to make substitutions within amino acid sequences of CDRs, antibody VH or VL domains and antigen binding sites are available in the art. Variants of antibody molecules disclosed herein may be produced and used in the present invention. Following the lead of computational chemistry in applying multivariate data analysis techniques to the structure/property-activity relationships (Wold, et al. Multivariate data analysis in chemistry. Chemometrics—Mathematics and Statistics in Chemistry (Ed.: B. Kowalski), D. Reidel Publishing Company, Dordrecht, Holland, 1984) quantitative activity-property relationships of antibodies can be derived using well-known mathematical techniques, such as statistical regression, pattern recognition and classification (Norman et al. Applied Regression Analysis. Wiley-Interscience; 3rd edition (April 1998); Kandel, Abraham & Backer, Eric. Computer-Assisted Reasoning in Cluster Analysis. Prentice Hall PTR, (May 11, 1995); Krzanowski, Wojtek. Principles of Multivariate Analysis: A User's Perspective (Oxford Statistical Science Series, No 22 (Paper)). Oxford University Press; (December 2000); Witten, Ian H. & Frank, Eibe. Data Mining: Practical Machine Learning Tools and Techniques with Java Implementations. Morgan Kaufmann; (Oct. 11, 1999); Denison David G. T. (Editor), Christopher C. Holmes, Bani K. Mallick, Adrian F. M. Smith. Bayesian Methods for Nonlinear Classification and Regression (Wiley Series in Probability and Statistics). John Wiley & Sons; (July 2002); Ghose, Arup K. & Viswanadhan, Vellarkad N. Combinatorial Library Design and Evaluation Principles, Software, Tools, and Applications in Drug Discovery). In some cases the properties of antibodies can be derived from empirical and theoretical models (for example, analysis of likely contact residues or calculated physicochemical property) of antibody sequence, functional and three-dimensional structures and these properties can be considered singly and in combination.

An antibody antigen-binding site composed of a VH domain and a VL domain is typically formed by six loops of polypeptide: three from the light chain variable domain (VL) and three from the heavy chain variable domain (VH). Analysis of antibodies of known atomic structure has elucidated relationships between the sequence and three-dimensional structure of antibody combining sites. These relationships imply that, except for the third region (loop) in VH domains, binding site loops have one of a small number of main-chain conformations: canonical structures. The canonical structure formed in a particular loop has been shown to be determined by its size and the presence of certain residues at key sites in both the loop and in framework regions.

This study of sequence-structure relationship can be used for prediction of those residues in an antibody of known sequence, but of an unknown three-dimensional structure, which are important in maintaining the three-dimensional structure of its CDR loops and hence maintain binding specificity. These predictions can be backed up by comparison of the predictions to the output from lead optimisation experiments. In a structural approach, a model can be created of the antibody molecule using any freely available or commercial package, such as WAM. A protein visualisation and analysis software package, such as Insight II (Accelrys, Inc.) or Deep View may then be used to evaluate possible substitutions at each position in the CDR. This information may then be used to make substitutions likely to have a minimal or beneficial effect on activity or confer other desirable properties.

The term “polypeptide fragment” as used herein refers to a polypeptide that has an amino-terminal and/or carboxy-terminal deletion, but where the remaining amino acid sequence identical to the corresponding positions in the naturally-occurring sequence deduced, for example, from a full-length cDNA sequence. Fragments typically are at least 5, 6, 8 or 10 amino acids long, preferably at least 14 amino acids long, more preferably at least 20 amino acids long, usually at least 50 amino acids long, and even more preferably at least 70 amino acids long. The term “analog” as used herein refers to polypeptides which are comprised of a segment of at least 25 amino acids that has substantial identity to a portion of a deduced amino acid sequence and which has at least one of the following properties: (1) specific binding to α5β1, under suitable binding conditions, (2) ability to block appropriate fibronectin/α5β1binding. Typically, polypeptide analogs comprise a conservative amino acid substitution (or addition or deletion) with respect to the naturally-occurring sequence. Analogs typically are at least 20 amino acids long, preferably at least 50 amino acids long or longer, and can often be as long as a full-length naturally-occurring polypeptide.

Peptide analogs are commonly used in the pharmaceutical industry as non-peptide drugs with properties analogous to those of the template peptide. These types of non-peptide compound are termed “peptide mimetics” or “peptidomimetics” (Fauchere, J. Adv. Drug Res. 15:29 (1986); Veber and Freidinger TINS p. 392 (1985); and Evans et al. J. Med. Chem. 30:1229 (1987), which are incorporated herein by reference). Such compounds are often developed with the aid of computerized molecular modeling. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a biochemical property or pharmacological activity), such as human antibody, but have one or more peptide linkages optionally replaced by a linkage selected from the group consisting of: —CH₂NH—, —CH₂S—, —CH₂—CH₂—, —CH═CH—(cis and trans), —COCH₂—, —CH(OH)C H₂—, and —CH₂SO—, by methods well known in the art. Systematic substitution of one or more amino acids of a consensus sequence with a D-amino acid of the same type (e.g., D-lysine in place of L-lysine) may be used to generate more stable peptides. In addition, constrained peptides comprising a consensus sequence or a substantially identical consensus sequence variation may be generated by methods known in the art (Rizo and Gierasch Ann. Rev. Biochem. 61:387 (1992), incorporated herein by reference); for example, by adding internal cysteine residues capable of forming intramolecular disulfide bridges which cyclize the peptide.

As used herein, the terms “antibody” and “antibodies” (immunoglobulins) encompass an oligoclonal antibody, a monoclonal antibody (including full-length monoclonal antibody), a polyclonal antibody, a chimeric antibody, a CDR-grafted antibody, a multi-specific antibody, a bi-specific antibody, a catalytic antibody, a chimeric antibody, a humanized antibody, a fully human antibody, an anti-idiotypic antibody and antibodies that can be labeled in soluble or bound form as well as fragments, variants or derivatives thereof, either alone or in combination with other amino acid sequences provided by known techniques. An antibody may be from any species. As used herein, the term “antibody” or “antibodies” refers to a polypeptide or group of polypeptides that are comprised of at least one binding domain that is formed from the folding of polypeptide chains having three-dimensional binding spaces with internal surface shapes and charge distributions complementary to the features of an antigenic determinant of an antigen. chain. Native antibodies are usually heterotetrameric glycoproteins of about 150,000 daltons, composed of two identical light (L) chains and two identical heavy (H) chains. Each light chain linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Light chains are classified as either lambda chains or kappa chains based on the amino acid sequence of the light chain constant region. The variable domain of a kappa light chain may also be denoted herein as VK. The term “variable region” may also be used to describe the variable domain of a heavy chain or light chain. Particular amino acid residues are believed to form an interface between the light and heavy chain variable domains. The variable regions of each light/heavy chain pair form an antibody binding site. Such antibodies may be derived from any mammal, including, but not limited to, humans, monkeys, pigs, horses, rabbits, dogs, cats, mice, etc.

The term “antibody” or “antibodies” includes binding fragments of the antibodies of the invention, exemplary fragments include single-chain Fvs (scFv), single-chain antibodies, single domain antibodies, domain antibodies, Fv fragments, Fab fragments, F(ab′) fragments, F(ab′)2 fragments, antibody fragments that exhibit the desired biological activity, disulfide-stabilised variable region (dsFv), dimeric variable region (Diabody), anti-idiotypic (anti-Id) antibodies (including, e.g., anti-Id antibodies to antibodies of the invention), intrabodies, linear antibodies, single-chain antibody molecules and multispecific antibodies formed from antibody fragments and epitope-binding fragments of any of the above. In particular, antibodies include immunoglobulin molecules and immunologically active fragments of immunoglobulin molecules, i.e., molecules that contain an antigen-binding site. Immunoglobulin molecules can be of any type (e.g., IgG, IgE, IgM, IgD, IgA and IgY), class (e.g., IgG1, IgG2, IgG3, IgG4, IgA1 and IgA2) or subclass.

Digestion of antibodies with the enzyme, papain, results in two identical antigen-binding fragments, known also as “Fab” fragments, and a “Fc” fragment, having no antigen-binding activity but having the ability to crystallize. Digestion of antibodies with the enzyme, pepsin, results in the a F(ab′)₂ fragment in which the two arms of the antibody molecule remain linked and comprise two-antigen binding sites. The F(ab′)₂ fragment has the ability to crosslink antigen.

“Fv” when used herein refers to the minimum fragment of an antibody that retains both antigen-recognition and antigen-binding sites. This region consists of a dimer of one heavy and one light chain variable domain in tight, non-covalent or covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

“Fab” when used herein refers to a fragment of an antibody that comprises the constant domain of the light chain and the CH1 domain of the heavy chain.

“dAb” when used herein refers to a fragment of an antibody that is the smallest functional binding unit of a human antibodies. A “dAb” is a single domain antibody and comprises either the variable domain of an antibody heavy chain (VH domain) or the variable domain of an antibody light chain (VL domain). Each dAb contains three of the six naturally occurring CDRs (Ward et al., Binding activities of a repertoire of single immunoglobulin variable domains secreted from Escherichia coli. Nature 341, 544-546 (1989); Holt, et al., Domain antibodies: protein for therapy, Trends Biotechnol. 21, 484-49 (2003)). With molecular weights ranging from 11 to 15 kDa, they are four times smaller than a fragment antigen binding (Fab)₂ and half the size of a single chain Fv (scFv) molecule.

“Camelid” when used herein refers to antibody molecules are composed of heavy-chain dimers which are devoid of light chains, but nevertheless have an extensive antigen-binding repertoire (Hamers-Casterman C, Atarhouch T, Muyldermans S, Robinson G, Hamers C, Songa E B, Bendahman N, Hamers R (1993) Naturally occurring antibodies devoid of light chains. Nature 363:446-448).

The term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

It has been shown that fragments of a whole antibody can perform the function of binding antigens. Examples of binding fragments are (Ward, E. S. et al., (1989) Nature 341, 544-546) the Fab fragment consisting of VL, VH, CL and CH1 domains; (McCafferty et al (1990) Nature, 348, 552-554) the Fd fragment consisting of the VH and CH1 domains; (Holt et al (2003) Trends in Biotechnology 21, 484-490) the Fv fragment consisting of the VL and VH domains of a single antibody; (iv) the dAb fragment (Ward, E. S. et al., Nature 341, 544-546 (1989), McCafferty et al (1990) Nature, 348, 552-554, Holt et al (2003) Trends in Biotechnology 21, 484-490], which consists of a VH or a VL domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al, (1988) Science, 242, 423-426, Huston et al, (1988) PNAS USA, 85, 5879-5883); (viii) bispecific single chain Fv dimers (PCT/US92/09965) and (ix) “diabodies”, multivalent or multispecific fragments constructed by gene fusion (WO 94/13804; Holliger, P. (1993) et al, Proc. Natl. Acad. Sci. USA 90 6444-6448). Fv, scFv or diabody molecules may be stabilised by the incorporation of disulphide bridges linking the VH and VL domains (Reiter, Y. et al, Nature Biotech, 14, 1239-1245, 1996). Minibodies comprising a scFv joined to a CH3 domain may also be made (Hu, S. et al, (1996) Cancer Res., 56, 3055-3061). Other examples of binding fragments are Fab′, which differs from Fab fragments by the addition of a few residues at the carboxyl terminus of the heavy chain CH1 domain, including one or more cysteines from the antibody hinge region, and Fab′-S H, which is a Fab′ fragment in which the cysteine residue(s) of the constant domains bear a free thiol group.

The term “variable” refers to the fact that certain portions of the variable domains differ extensively in sequence among antibodies and are responsible for the binding specificity of each particular antibody for its particular antigen. However, the variability is not evenly distributed through the variable domains of antibodies. It is concentrated in segments called Complementarity Determining Regions (CDRs) both in the light chain and the heavy chain variable domains. The more highly conserved portions of the variable domains are called the framework regions (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies (see, Kabat et al., Sequences of Proteins of Immunological Interest, 5th Ed. Public Health Service, National Institutes of Health, Bethesda, Md. (1991)). The constant domains are generally not involved directly in antigen binding, but may influence antigen binding affinity and may exhibit various effector functions, such as participation of the antibody in ADCC, CDC, and/or apoptosis.

The term “hypervariable region” when used herein refers to the amino acid residues of an antibody which are associated with its binding to antigen. The hypervariable regions encompass the amino acid residues of the “complementarity determining regions” or “CDRs” (e.g., residues 24-34 (L1), 50-56 (L2) and 89-97 (L3) of the light chain variable domain and residues 31-35 (H1), 50-65 (H2) and 95-102 (H3) of the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g., residues 26-32 (L1 ), 50-52 (L2) and 91-96 (L3) in the light chain variable domain and 26-32 (H1), 53-55 (H2) and 96-101 (H3) in the heavy chain variable domain; Chothia and Lesk, J. Mol. Biol., 196:901-917 (1987)). “Framework” or “FR” residues are those variable domain residues flanking the CDRs. FR residues are present in chimeric, humanized, human, domain antibodies, diabodies, vaccibodies, linear antibodies, and bispecific antibodies.

As used herein, targeted binding agent, targeted binding protein, specific binding protein and like terms refer to an antibody, or binding fragment thereof that preferentially binds to a target site. In one embodiment, the targeted binding agent is specific for only one target site. In other embodiments, the targeted binding agent is specific for more than one target site. In one embodiment, the targeted binding agent may be a monoclonal antibody and the target site may be an epitope.

“Binding fragments” of an antibody are produced by recombinant DNA techniques, or by enzymatic or chemical cleavage of intact antibodies. Binding fragments include Fab, Fab′, F(ab′)₂, Fv, dAb and single-chain antibodies. An antibody other than a “bispecific” or “bifunctional” antibody is understood to have each of its binding sites identical. An antibody substantially inhibits adhesion of a receptor to a counter-receptor when an excess of antibody reduces the quantity of receptor bound to counter-receptor by at least about 20%, 40%, 60% or 80%, and more usually greater than about 85% (as measured in an in vitro competitive binding assay).

The term “epitope” includes any protein determinant capable of specific binding to an immunoglobulin or T-cell receptor. Epitopic determinants usually consist of chemically active surface groupings of molecules such as amino acids or sugar side chains and may, but not always, have specific three-dimensional structural characteristics, as well as specific charge characteristics. An antibody is said to specifically bind an antigen when the dissociation constant is ≦1 μM, preferably 100 nM and most preferably 10 nM.

The term “Geomean” (also known as geometric mean), refers to the average of the logarithmic values of a data set, converted back to a base 10 number. This requires there to be at least two measurements, e.g. at least 2, preferably at least 5, more preferably at least 10 replicate. The person skilled in the art will appreciate that the greater the number of replicates the more robust the geomean value will be. The choice of replicate number can be left to the discretion of the person skilled in the art.

The term “agent” is used herein to denote a chemical compound, a mixture of chemical compounds, a biological macromolecule, or an extract made from biological materials.

“Active” or “activity” in regard to an α5β1 polypeptide refers to a portion of an α5β1 polypeptide that has a biological or an immunological activity of a native α5β1 polypeptide. “Biological” when used herein refers to a biological function that results from the activity of the native α5β1 polypeptide. A preferred α5β1 biological activity includes, for example, α5β1 induced cell adhesion and invasion and/or angiogenesis and/or proliferation.

“Mammal” when used herein refers to any animal that is considered a mammal Preferably, the mammal is human

“Animal” when used herein encompasses animals considered a mammal Preferably the animal is human

The term “mAb” refers to monoclonal antibody.

“Liposome” when used herein refers to a small vesicle that may be useful for delivery of drugs that may include the α5β1 polypeptide of the invention or antibodies to such an α5β1 polypeptide to a mammal.

“Label” or “labeled” as used herein refers to the addition of a detectable moiety to a polypeptide, for example, a radiolabel, fluorescent label, enzymatic label chemiluminescent labeled or a biotinyl group. Radioisotopes or radionuclides may include ³H, ¹⁴C, ¹⁵N, ³⁵s, ⁹⁰Y, ⁹⁹Tc, ¹¹¹In, ¹²⁵I, ¹³¹I, fluorescent labels may include rhodamine, lanthanide phosphors or FITC and enzymatic labels may include horseradish peroxidase, β-galactosidase, luciferase, alkaline phosphatase.

Additional labels include, by way of illustration and not limitation: enzymes, such as glucose-6-phosphate dehydrogenase (“G6PDH”), alpha-D-galactosidase, glucose oxydase, glucose amylase, carbonic anhydrase, acetylcholinesterase, lysozyme, malate dehydrogenase and peroxidase; dyes; additional fluorescent labels or fluorescers include, such as fluorescein and its derivatives, fluorochrome, GFP (GFP for “Green Fluorescent Protein”), dansyl, umbelliferone, phycoerythrin, phycocyanin, allophycocyanin, o-phthaldehyde, and fluorescamine; fluorophores such as lanthanide cryptates and chelates e.g. Europium etc (Perkin Elmer and Cis Biointernational); chemoluminescent labels or chemiluminescers, such as isoluminol, luminol and the dioxetanes; sensitisers; coenzymes; enzyme substrates; particles, such as latex or carbon particles; metal sol; crystallite; liposomes; cells, etc., which may be further labelled with a dye, catalyst or other detectable group; molecules such as biotin, digoxygenin or 5-bromodeoxyuridine; toxin moieties, such as for example a toxin moiety selected from a group of Pseudomonas exotoxin (PE or a cytotoxic fragment or mutant thereof), Diptheria toxin or a cytotoxic fragment or mutant thereof, a botulinum toxin A, B, C, D, E or F, ricin or a cytotoxic fragment thereof e.g. ricin A, abrin or a cytotoxic fragment thereof, saporin or a cytotoxic fragment thereof, pokeweed antiviral toxin or a cytotoxic fragment thereof and bryodin 1 or a cytotoxic fragment thereof.

The term “pharmaceutical agent or drug” as used herein refers to a chemical compound or composition capable of inducing a desired therapeutic effect when properly administered to a patient. Other chemistry terms herein are used according to conventional usage in the art, as exemplified by The McGraw-Hill Dictionary of Chemical Terms (Parker, S., Ed., McGraw-Hill, San Francisco (1985)), (incorporated herein by reference).

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

The term “patient” includes human and veterinary subjects.

“Antibody-dependent cell-mediated cytotoxicity” and “ADCC” refer to a cell-mediated reaction in which non-specific cytotoxic cells that express Ig Fc receptors (FcRs) (e.g. Natural Killer (NK) cells, monocytes, neutrophils, and macrophages) recognise bound antibody on a target cell and subsequently cause lysis of the target cell. The primary cells for mediating ADCC, NK cells, express FcγRIII only, whereas monocytes express FcγRI, FcγRII and FcγRIII. FcRs expression on hematopoietic cells is summarised in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362, or 5,821,337 can be performed. Useful effector cells for such assays include peripheral blood mononuclear cells (PBMC) and Natural Killer (NK) cells. Alternatively, or additionally, ADCC activity of the molecule of interest can be assessed in vivo, e.g., in an animal model such as that disclosed in Clynes et al. PNAS (USA) 95:652-656 (1988). “Complement dependent cytotoxicity” and “CDC” refer to the mechanism by which antibodies carry out their cell-killing function. It is initiated by the binding of C1q, a constituent of the first component of complement, to the Fc domain of Igs, IgG or IgM, which are in complex with antigen (Hughs-Jones, N. C., and B. Gardner. 1979. Mol. Immunol. 16:697). C1q is a large, structurally complex glycoprotein of ˜410 kDa present in human serum at a concentration of 70 μg/ml (Cooper, N. R. 1985. Adv. Immunol. 37:151). Together with two serine proteases, C1r and C1s, C1q forms the complex C1, the first component of complement. At least two of the N-terminal globular heads of C1q must be bound to the Fc of Igs for C1 activation, hence for initiation of the complement cascade (Cooper, N. R. 1985. Adv. Immunol. 37:151).

The term “antibody half-life” as used herein means a pharmacokinetic property of an antibody that is a measure of the mean survival time of antibody molecules following their administration. Antibody half-life can be expressed as the time required to eliminate 50 percent of a known quantity of immunoglobulin from the patient's body or a specific compartment thereof, for example, as measured in serum or plasma, i.e., circulating half-life, or in other tissues. Half-life may vary from one immunoglobulin or class of immunoglobulin to another. In general, an increase in antibody half-life results in an increase in mean residence time (MRT) in circulation for the antibody administered.

The term “isotype” refers to the classification of an antibody's heavy or light chain constant region. The constant domains of antibodies are not involved in binding to antigen, but exhibit various effector functions. Depending on the amino acid sequence of the heavy chain constant region, a given human antibody or immunoglobulin can be assigned to one of five major classes of immunoglobulins: IgA, IgD, IgE, IgG, and IgM. Several of these classes may be further divided into subclasses (isotypes), e.g., IgG1 (gamma 1), IgG2 (gamma 2), IgG3 (gamma 3), and IgG4 (gamma 4), and IgA1 and IgA2. The heavy chain constant regions that correspond to the different classes of immunoglobulins are called α, δ, ε, γ, and μ, respectively. The structures and three-dimensional configurations of different classes of immunoglobulins are well-known. Of the various human immunoglobulin classes, only human IgG1, IgG2, IgG3, IgG4, and IgM are known to activate complement. Human IgG1 and IgG3 are known to mediate in humans. Human light chain constant regions may be classified into two major classes, kappa and lambda.

If desired, the isotype of an antibody that specifically binds α5β1 can be switched, for example to take advantage of a biological property of a different isotype. For example, in some circumstances it can be desirable in connection with the generation of antibodies as therapeutic antibodies against α5β1 that the antibodies be capable of fixing complement and participating in complement-dependent cytotoxicity (CDC). There are a number of isotypes of antibodies that are capable of the same, including, without limitation, the following: murine IgM, murine IgG2a, murine IgG2b, murine IgG3, human IgM, human IgA, human IgG1, and human IgG3. In other embodiments it can be desirable in connection with the generation of antibodies as therapeutic antibodies against α5β1 that the antibodies be capable of binding Fc receptors on effector cells and participating in antibody-dependent cytotoxicity (ADCC). There are a number of isotypes of antibodies that are capable of the same, including, without limitation, the following: murine IgG2a, murine IgG2b, murine IgG3, human IgG1, and human IgG3. It will be appreciated that antibodies that are generated need not initially possess such an isotype but, rather, the antibody as generated can possess any isotype and the antibody can be isotype switched thereafter using conventional techniques that are well known in the art. Such techniques include the use of direct recombinant techniques (see e.g., U.S. Pat. No. 4,816,397), cell-cell fusion techniques (see e.g., U.S. Pat. Nos. 5,916,771 and 6,207,418), among others.

By way of example, the anti-α5β1 antibodies discussed herein are fully human antibodies. If an antibody possessed desired binding to α5β1, it could be readily isotype switched to generate a human IgM, human IgG1, or human IgG3 isotype, while still possessing the same variable region (which defines the antibody's specificity and some of its affinity). Such molecule would then be capable of fixing complement and participating in CDC and/or be capable of binding to Fc receptors on effector cells and participating in ADCC.

“Whole blood assays” use unfractionated blood as a source of natural effectors. Blood contains complement in the plasma, together with FcR-expressing cellular effectors, such as polymorphonuclear cells (PMNs) and mononuclear cells (MNCs). Thus, whole blood assays allow simultaneous evaluation of the synergy of both ADCC and CDC effector mechanisms in vitro.

A “therapeutically effective” amount as used herein is an amount that provides some improvement or benefit to the subject. Stated in another way, a “therapeutically effective” amount is an amount that provides some alleviation, mitigation, and/or decrease in at least one clinical symptom. Clinical symptoms associated with the disorders that can be treated by the methods of the invention are well-known to those skilled in the art. Further, those skilled in the art will appreciate that the therapeutic effects need not be complete or curative, as long as some to benefit is provided to the subject.

The term “and/or” as used herein is to be taken as specific disclosure of each of the two specified features or components with or without the other. For example “A and/or B” is to be taken as specific disclosure of each of (i) A, (ii) B and (iii) A and B, just as if each is set out individually herein.

### Antibody Structure

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody binding site.

Thus, an intact antibody has two binding sites. Except in bifunctional or bispecific antibodies, the two binding sites are the same.

The chains all exhibit the same general structure of relatively conserved framework regions (FR) joined by three hyper variable regions, also called CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987); Chothia et al. Nature 342:878-883 (1989).

A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann Clin. Exp. Immunol. 79: 315-321 (1990), Kostelny et al. J. Immunol. 148:1547-1553 (1992). Bispecific antibodies do not exist in the form of fragments having a single binding site (e.g., Fab, Fab′, and Fv).

Typically, a VH domain is paired with a VL domain to provide an antibody antigen-binding site, although a VH or VL domain alone may be used to bind antigen. The VH domain (see Table 12) may be paired with the VL domain (see Table 13), so that an antibody antigen-binding site is formed comprising both the VH and VL domains.

### Human Antibodies and Humanization of Antibodies

Targeted binding agents of the invention include human antibodies. Human antibodies avoid some of the problems associated with antibodies that possess murine or rat variable and/or constant regions. The presence of such murine or rat derived proteins can lead to the rapid clearance of the antibodies or can lead to the generation of an immune response against the antibody by a patient. In order to avoid the utilization of murine or rat derived antibodies, fully human antibodies can be generated through the introduction of functional human antibody loci into a rodent, other mammal or animal so that the rodent, other mammal or animal produces fully human antibodies.

One method for generating fully human antibodies is through the use of XenoMouse® strains of mice that have been engineered to contain up to but less than 1000 kb-sized germline configured fragments of the human heavy chain locus and kappa light chain locus. See Mendez et al. Nature Genetics 15:146-156 (1997) and Green and Jakobovits J. Exp. Med. 188:483-495 (1998). The XenoMouse® strains are available from Amgen, Inc. (Fremont, Calif., U.S.A).

Such mice, then, are capable of producing human immunoglobulin molecules and antibodies and are deficient in the production of murine immunoglobulin molecules and antibodies. Technologies utilised for achieving the same are disclosed in U.S. patent application Ser. No. 08/759,620, filed Dec. 3, 1996 and International Patent Application Nos. WO 98/24893, published Jun. 11, 1998 and WO 00/76310, published Dec. 21, 2000, the disclosures of which are hereby incorporated by reference. See also Mendez et al. Nature Genetics 15:146-156 (1997), the disclosure of which is hereby incorporated by reference.

The production of the XenoMouse® strains of mice is further discussed and delineated in U.S. patent application Ser. Nos. 07/466,008, filed Jan. 12, 1990, 07/610,515, filed Nov. 8, 1990, 07/919,297, filed Jul. 24, 1992, 07/922,649, filed Jul. 30, 1992, 08/031,801, filed Mar. 15, 1993, 08/112,848, filed Aug. 27, 1993, 08/234,145, filed Apr. 28, 1994, 08/376,279, filed Jan. 20, 1995, 08/430, 938, filed Apr. 27, 1995, 08/464,584, filed Jun. 5, 1995, 08/464,582, filed Jun. 5, 1995, 08/463,191, filed Jun. 5, 1995, 08/462,837, filed Jun. 5, 1995, 08/486,853, filed Jun. 5, 1995, 08/486,857, filed Jun. 5, 1995, 08/486,859, filed Jun. 5, 1995, 08/462,513, filed Jun. 5, 1995, 08/724,752, filed Oct. 2, 1996, 08/759,620, filed Dec. 3, 1996, U.S. Publication 2003/0093820, filed Nov. 30, 2001 and U.S. Pat. Nos. 6,162,963, 6,150,584, 6,114,598, 6,075,181, and 5,939,598 and Japanese Patent Nos. 3 068 180 B2, 3 068 506 B2, and 3 068 507 B2. See also European Patent No., EP 0 463 151 B1, grant published Jun. 12, 1996, International Patent Application No., WO 94/02602, published Feb. 3, 1994, International Patent Application No., WO 96/34096, published Oct. 31, 1996, WO 98/24893, published Jun. 11, 1998, WO 00/76310, published Dec. 21, 2000. The disclosures of each of the above-cited patents, applications, and references are hereby incorporated by reference in their entirety.

In an alternative approach, others, including GenPharm International, Inc., have utilised a “minilocus” approach. In the minilocus approach, an exogenous Ig locus is mimicked through the inclusion of pieces (individual genes) from the Ig locus. Thus, one or more V_{H} genes, one or more D_{H} genes, one or more J_{H} genes, a mu constant region, and usually a second constant region (preferably a gamma constant region) are formed into a construct for insertion into an animal. This approach is described in U.S. Pat. No. 5,545,807 to Surani et al. and U.S. Pat. Nos. 5,545,806, 5,625,825, 5,625,126, 5,633,425, 5,661,016, 5,770,429, 5,789,650, 5,814,318, 5,877,397, 5,874,299, and 6,255,458 each to Lonberg and Kay, U.S. Pat. Nos. 5,591,669 and 6,023.010 to Krimpenfort and Berns, U.S. Pat. Nos. 5,612,205, 5,721,367, and 5,789,215 to Berns et al., and U.S. Pat. No. 5,643,763 to Choi and Dunn, and GenPharm International U.S. patent application Ser. Nos. 07/574,748, filed Aug. 29, 1990, 07/575,962, filed Aug. 31, 1990, 07/810,279, filed Dec. 17, 1991, 07/853,408, filed Mar. 18, 1992, 07/904,068, filed Jun. 23, 1992, 07/990,860, filed Dec. 16, 1992, 08/053,131, filed Apr. 26, 1993, 08/096,762, filed Jul. 22, 1993, 08/155,301, filed Nov. 18, 1993, 08/161,739, filed Dec. 3, 1993, 08/165,699, filed Dec. 10, 1993, 08/209,741, filed Mar. 9, 1994, the disclosures of which are hereby incorporated by reference. See also European Patent No. 0 546 073 B1, International Patent Application Nos. WO 92/03918, WO 92/22645, WO 92/22647, WO 92/22670, WO 93/12227, WO 94/00569, WO 94/25585, WO 96/14436, WO 97/13852, and WO 98/24884 and U.S. Pat. No. 5,981,175, the disclosures of which are hereby incorporated by reference in their entirety. See further Taylor et al., 1992, Chen et al., 1993, Tuaillon et al., 1993, Choi et al., 1993, Lonberg et al., (1994), Taylor et al., (1994), and Tuaillon et al., (1995), Fishwild et al., (1996), the disclosures of which are hereby incorporated by reference in their entirety.

Kirin has also demonstrated the generation of human antibodies from mice in which, through microcell fusion, large pieces of chromosomes, or entire chromosomes, have been introduced. See European Patent Application Nos. 773 288 and 843 961, the disclosures of which are hereby incorporated by reference. Additionally, KM™—mice, which are the result of cross-breeding of Kirin's Tc mice with Medarex's minilocus (Humab) mice have been generated. These mice possess the human IgH transchromosome of the Kirin mice and the kappa chain transgene of the Genpharm mice (Ishida et al., Cloning Stem Cells, (2002) 4:91-102).

Human antibodies can also be derived by in vitro methods. Suitable examples include but are not limited to phage display (Medimmune, Morphosys, Dyax, Biosite/Medarex, Xoma, Symphogen, Alexion (formerly Proliferon), Affimed) ribosome display (Medimmune), yeast display, and the like.

### Preparation of Antibodies

Antibodies, as described herein, were prepared through the utilization of the XenoMouse® technology, as described below. Such mice are capable of producing human immunoglobulin molecules and antibodies and are deficient in the production of murine immunoglobulin molecules and antibodies. Technologies utilised for achieving the same are disclosed in the patents, applications, and references disclosed in the background section herein. In particular, however, a preferred embodiment of transgenic production of mice and antibodies therefrom is disclosed in U.S. patent application Ser. No. 08/759,620, filed Dec. 3, 1996 and International Patent Application Nos. WO 98/24893, published Jun. 11, 1998 and WO 00/76310, published Dec. 21, 2000, the disclosures of which are hereby incorporated by reference. See also Mendez et al. Nature Genetics 15:146-156 (1997), the disclosure of which is hereby incorporated by reference.

Through the use of such technology, fully human monoclonal antibodies to a variety of antigens have been produced. Essentially, XenoMouse® lines of mice are immunised with an antigen of interest (e.g. α5β1), lymphatic cells (such as B-cells) are recovered from the hyper-immunised mice, and the recovered lymphocytes are fused with a myeloid-type cell line to prepare immortal hybridoma cell lines. These hybridoma cell lines are screened and selected to identify hybridoma cell lines that produced antibodies specific to the antigen of interest. Provided herein are methods for the production of multiple hybridoma cell lines that produce antibodies specific to α5β1. Further, provided herein are characterisation of the antibodies produced by such cell lines, including nucleotide and amino acid sequence analyses of the heavy and light chains of such antibodies.

Alternatively, instead of being fused to myeloma cells to generate hybridomas, B cells can be directly assayed. For example, CD19+ B cells can be isolated from hyperimmune XenoMouse® mice and allowed to proliferate and differentiate into antibody-secreting plasma cells. Antibodies from the cell supernatants are then screened by ELISA for reactivity against the α5β1 immunogen. The supernatants might also be screened for immunoreactivity against fragments of α5β1 to further map the different antibodies for binding to domains of functional interest on α5β1. The antibodies may also be screened other related human endoglycosidases and against the rat, the mouse, and non-human primate, such as Cynomolgus monkey, orthologues of α5β1, the last to determine species cross-reactivity. B cells from wells containing antibodies of interest may be immortalised by various methods including fusion to make hybridomas either from individual or from pooled wells, or by infection with EBV or transfection by known immortalising genes and then plating in suitable medium. Alternatively, single plasma cells secreting antibodies with the desired specificities are then isolated using an α5β1-specific hemolytic plaque assay (see for example Babcook et al., Proc. Natl. Acad. Sci. USA 93:7843-48 (1996)). Cells targeted for lysis are preferably sheep red blood cells (SRBCs) coated with the α5β1 antigen.

In the presence of a B-cell culture containing plasma cells secreting the immunoglobulin of interest and complement, the formation of a plaque indicates specific α5β1-mediated lysis of the sheep red blood cells surrounding the plasma cell of interest. The single antigen-specific plasma cell in the center of the plaque can be isolated and the genetic information that encodes the specificity of the antibody is isolated from the single plasma cell. Using reverse-transcription followed by PCR (RT-PCR), the DNA encoding the heavy and light chain variable regions of the antibody can be cloned. Such cloned DNA can then be further inserted into a suitable expression vector, preferably a vector cassette such as a pcDNA, more preferably such a pcDNA vector containing the constant domains of immunglobulin heavy and light chain. The generated vector can then be transfected into host cells, e.g., HEK293 cells, CHO cells, and cultured in conventional nutrient media modified as appropriate for inducing transcription, selecting transformants, or amplifying the genes encoding the desired sequences.

As will be appreciated, antibodies that specifically bind α5β1 can be expressed in cell lines other than hybridoma cell lines. Sequences encoding particular antibodies can be used to transform a suitable mammalian host cell. Transformation can be by any known method for introducing polynucleotides into a host cell, including, for example packaging the polynucleotide in a virus (or into a viral vector) and transducing a host cell with the virus (or vector) or by transfection procedures known in the art, as exemplified by U.S. Pat. Nos. 4,399,216, 4,912,040, 4,740,461, and 4,959,455 (which patents are hereby incorporated herein by reference). The transformation procedure used depends upon the host to be transformed. Methods for introducing heterologous polynucleotides into mammalian cells are well known in the art and include dextran-mediated transfection, calcium phosphate precipitation, polybrene mediated transfection, protoplast fusion, electroporation, encapsulation of the polynucleotide(s) in liposomes, and direct microinjection of the DNA into nuclei.

Mammalian cell lines available as hosts for expression are well known in the art and include many immortalized cell lines available from the American Type Culture Collection (ATCC), including but not limited to Chinese hamster ovary (CHO) cells, HeLa cells, baby hamster kidney (BHK) cells, monkey kidney cells (COS), human hepatocellular carcinoma cells (e.g., Hep G2), human epithelial kidney 293 cells, and a number of other cell lines (Chadd, H. E. and Chamow, S. M., (2001) Curr Opin in Biotech. 12: 188-194; Andersen, D. C. and Krummen, L, (2002) Curr Opin in Biotech. 13:117; Larrick, J. W., and Thomas, D. W., (2001) Curr Opin in Biotech. 12: 411-418). Cell lines of particular preference are selected through determining which cell lines have high expression levels and produce antibodies with constitutive α5β1 binding properties.

In the cell-cell fusion technique, a myeloma, CHO cell or other cell line is prepared that possesses a heavy chain with any desired isotype and another myeloma, CHO cell or other cell line is prepared that possesses the light chain. Such cells can, thereafter, be fused and a cell line expressing an intact antibody can be isolated.

Accordingly, as antibody candidates are generated that meet desired “structural” attributes as discussed above, they can generally be provided with at least certain of the desired “functional” attributes through isotype switching.

### Therapeutic Administration and Formulations

Embodiments of the invention include sterile pharmaceutical formulations of anti-α5β1 antibodies that are useful as treatments for diseases. Such formulations would inhibit the binding of a native α5β1-specific ligand such as, for example, fibronectin, to α5β1, thereby effectively treating pathological conditions where, for example, serum or tissue α5β1 expression is abnormally elevated. Anti-α5β1 antibodies preferably possess adequate affinity to potently inhibit native α5β1-specific ligands such as, for example, fibronectin, and preferably have an adequate duration of action to allow for infrequent dosing in humans. A prolonged duration of action will allow for less frequent and more convenient dosing schedules by alternate parenteral routes such as subcutaneous or intramuscular injection.

Sterile formulations can be created, for example, by filtration through sterile filtration membranes, prior to or following lyophilization and reconstitution of the antibody. The antibody ordinarily will be stored in lyophilized form or in solution. Therapeutic antibody compositions generally are placed into a container having a sterile access port, for example, an intravenous solution bag or vial having an adapter that allows retrieval of the formulation, such as a stopper pierceable by a hypodermic injection needle.

The route of antibody administration is in accord with known methods, e.g., injection or infusion by intravenous, intraperitoneal, intracerebral, intramuscular, intraocular, intraarterial, intrathecal, inhalation or intralesional routes, direct injection to a tumour site, or by sustained release systems as noted below. The antibody is preferably administered continuously by infusion or by bolus injection.

An effective amount of antibody to be employed therapeutically will depend, for example, upon the therapeutic objectives, the route of administration, and the condition of the patient. Accordingly, it is preferred that the therapist titer the dosage and modify the route of administration as required to obtain the optimal therapeutic effect. Typically, the clinician will administer antibody until a dosage is reached that achieves the desired effect. The progress of this therapy is easily monitored by conventional assays or by the assays described herein.

Antibodies, as described herein, can be prepared in a mixture with a pharmaceutically acceptable carrier. This therapeutic composition can be administered intravenously or through the nose or lung, preferably as a liquid or powder aerosol (lyophilized). The composition may also be administered parenterally or subcutaneously as desired. When administered systemically, the therapeutic composition should be sterile, pyrogen-free and in a parenterally acceptable solution having due regard for pH, isotonicity, and stability. These conditions are known to those skilled in the art. Briefly, dosage formulations of the compounds described herein are prepared for storage or administration by mixing the compound having the desired degree of purity with pharmaceutically acceptable carriers, excipients, or stabilizers. Such materials are non-toxic to the recipients at the dosages and concentrations employed, and include buffers such as TRIS HCl, phosphate, citrate, acetate and other organic acid salts; antioxidants such as ascorbic acid; low molecular weight (less than about ten residues) peptides such as polyarginine, proteins, such as serum albumin, gelatin, or immunoglobulins; hydrophilic polymers such as polyvinylpyrrolidinone; amino acids such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium and/or nonionic surfactants such as TWEEN, PLURONICS or polyethyleneglycol.

Sterile compositions for injection can be formulated according to conventional pharmaceutical practice as described in Remington: The Science and Practice of Pharmacy (20^{th} ed, Lippincott Williams & Wilkens Publishers (2003)). For example, dissolution or suspension of the active compound in a pharmaceutically acceptable carrier such as water or naturally occurring vegetable oil like sesame, peanut, or cottonseed oil or a synthetic fatty vehicle like ethyl oleate or the like may be desired. Buffers, preservatives, antioxidants and the like can be incorporated according to accepted pharmaceutical practice.

Suitable examples of sustained-release preparations include semipermeable matrices of solid hydrophobic polymers containing the polypeptide, which matrices are in the form of shaped articles, films or microcapsules. Examples of sustained-release matrices include polyesters, hydrogels (e.g., poly(2-hydroxyethyl-methacrylate) as described by Langer et al., J. Biomed Mater. Res., (1981) 15:167-277 and Langer, Chem. Tech., (1982) 12:98-105, or poly(vinylalcohol)), polylactides (U.S. Pat. No. 3,773,919, EP 58,481), copolymers of L-glutamic acid and gamma ethyl-L-glutamate (Sidman et al., Biopolymers, (1983) 22:547-556), non-degradable ethylene-vinyl acetate (Langer et al., supra), degradable lactic acid-glycolic acid copolymers such as the LUPRON Depot™ (injectable microspheres composed of lactic acid-glycolic acid copolymer and leuprolide acetate), and poly-D-(−)-3-hydroxybutyric acid (EP 133,988).

While polymers such as ethylene-vinyl acetate and lactic acid-glycolic acid enable release of molecules for over 100 days, certain hydrogels release proteins for shorter time periods. When encapsulated proteins remain in the body for a long time, they may denature or aggregate as a result of exposure to moisture at 37° C., resulting in a loss of biological activity and possible changes in immunogenicity. Rational strategies can be devised for protein stabilization depending on the mechanism involved. For example, if the aggregation mechanism is discovered to be intermolecular S—S bond formation through disulfide interchange, stabilization may be achieved by modifying sulfhydryl residues, lyophilizing from acidic solutions, controlling moisture content, using appropriate additives, and developing specific polymer matrix compositions.

Sustained-released compositions also include preparations of crystals of the antibody suspended in suitable formulations capable of maintaining crystals in suspension. These preparations when injected subcutaneously or intraperitonealy can produce a sustained release effect. Other compositions also include liposomally entrapped antibodies. Liposomes containing such antibodies are prepared by methods known per se: U.S. Pat. No. DE 3,218,121; Epstein et al., Proc. Natl. Acad. Sci. USA, (1985) 82:3688-3692; Hwang et al., Proc. Natl. Acad. Sci. USA, (1980) 77:4030-4034; EP 52,322; EP 36,676; EP 88,046; EP 143,949; 142,641; Japanese patent application 83-118008; U.S. Pat. Nos. 4,485,045 and 4,544,545; and EP 102,324.

The dosage of the antibody formulation for a given patient will be determined by the attending physician taking into consideration various factors known to modify the action of drugs including severity and type of disease, body weight, sex, diet, time and route of administration, other medications and other relevant clinical factors. Therapeutically effective dosages may be determined by either in vitro or in vivo methods.

An effective amount of the antibodies, described herein, to be employed therapeutically will depend, for example, upon the therapeutic objectives, the route of administration, and the condition of the patient. Accordingly, it is preferred for the therapist to titer the dosage and modify the route of administration as required to obtain the optimal therapeutic effect. A typical daily dosage might range from about 0.0001 mg/kg, 0.001 mg/kg, 0.01 mg/kg, 0.1 mg/kg, 1 mg/kg, 10 mg/kg to up to 100 mg/kg, 1000 mg/kg, 10000 mg/kg or more, of the patient's body weight depending on the factors mentioned above. The dosage may be between 0.0001 mg/kg and 20 mg/kg, 0.0001 mg/kg and 10 mg/kg, 0.0001 mg/kg and 5 mg/kg, 0.0001 and 2 mg/kg, 0.0001 and 1 mg/kg, 0.0001 mg/kg and 0.75 mg/kg, 0.0001 mg/kg and 0.5 mg/kg, 0.0001 mg/kg to 0.25 mg/kg, 0.0001 to 0.15 mg/kg, 0.0001 to 0.10 mg/kg, 0.001 to 0.5 mg/kg, 0.01 to 0.25 mg/kg or 0.01 to 0.10 mg/kg of the patient's body weight depending on the factors mentioned above. Typically, the clinician will administer the therapeutic antibody until a dosage is reached that achieves the desired effect. The progress of this therapy is easily monitored by conventional assays or as described herein.

Doses of antibodies of the invention may be repeated and the administrations may be separated by at least 1 day, 2 days, 3 days, 5 days, 10 days, 15 days, 30 days, 45 days, 2 months, 75 days, 3 months, or at least 6 months.

It will be appreciated that administration of therapeutic entities in accordance with the compositions and methods herein will be administered with suitable carriers, excipients, and other agents that are incorporated into formulations to provide improved transfer, delivery, tolerance, and the like. These formulations include, for example, powders, pastes, ointments, jellies, waxes, oils, lipids, lipid (cationic or anionic) containing vesicles (such as Lipofectin™) DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol. Pharmacol. 32(2):210-8 (2000), Wang W. “Lyophilization and development of solid protein pharmaceuticals.” Int. J. Pharm. 203(1-2):1-60 (2000), Charman W N “Lipids, lipophilic drugs, and oral drug delivery-some emerging concepts.” J Pharm Sci 89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

### Design and Generation of Other Therapeutics

In accordance with the present invention and based on the activity of the antibodies that are produced and characterized herein with respect to α5β1, the design of other therapeutic modalities beyond antibody moieties is facilitated. Such modalities include, without limitation, advanced antibody therapeutics, such as bispecific antibodies, immunotoxins, and radiolabeled therapeutics, single domain antibodies, antibody fragments, such as a Fab, Fab′, F(ab′)₂, Fv or dAb, generation of peptide therapeutics, α5β1 binding domains in novel scaffolds, gene therapies, particularly intrabodies, antisense therapeutics, and small molecules.

An antigen binding site may be provided by means of arrangement of CDRs on non-antibody protein scaffolds, such as fibronectin or cytochrome B etc. (Haan & Maggos (2004) BioCentury, 12(5): A1-A6; Koide et al. (1998) Journal of Molecular Biology, 284: 1141-1151; Nygren et al. (1997) Current Opinion in Structural Biology, 7: 463-469) or by randomising or mutating amino acid residues of a loop within a protein scaffold to confer binding specificity for a desired target. Scaffolds for engineering novel binding sites in proteins have been reviewed in detail by Nygren et al. (Nygren et al. (1997) Current Opinion in Structural Biology, 7: 463-469). Protein scaffolds for antibody mimics are disclosed in WO/0034784, which is herein incorporated by reference in its entirety, in which the inventors describe proteins (antibody mimics) that include a fibronectin type III domain having at least one randomised loop. A suitable scaffold into which to graft one or more CDRs, e.g. a set of HCDRs, may be provided by any domain member of the immunoglobulin gene superfamily. The scaffold may be a human or non-human protein. An advantage of a non-antibody protein scaffold is that it may provide an antigen-binding site in a scaffold molecule that is smaller and/or easier to manufacture than at least some antibody molecules. Small size of a binding member may confer useful physiological properties, such as an ability to enter cells, penetrate deep into tissues or reach targets within other structures, or to bind within protein cavities of the target antigen. Use of antigen binding sites in non-antibody protein scaffolds is reviewed in Wess, 2004 (Wess, L. In: BioCentury, The Bernstein Report on BioBusiness, 12(42), A1-A7, 2004). Typical are proteins having a stable backbone and one or more variable loops, in which the amino acid sequence of the loop or loops specifically or randomly mutated to create an antigen-binding site that binds the target antigen. Such proteins include the IgG-binding domains of protein A from S. aureus, transferrin, albumin, tetranectin, fibronectin (e.g. 10th fibronectin type III domain), lipocalins as well as gamma-crystalline and other Affilin™ scaffolds (Scil Proteins). Examples of other approaches include synthetic “Microbodies” based on cyclotides—small proteins having intra-molecular disulphide bonds, Microproteins (Versabodies™, Amunix) and ankyrin repeat proteins (DARPins, Molecular Partners).

In addition to antibody sequences and/or an antigen-binding site, a targeted binding agent according to the present invention may comprise other amino acids, e.g. forming a peptide or polypeptide, such as a folded domain, or to impart to the molecule another functional characteristic in addition to ability to bind antigen. Targeted binding agents of the invention may carry a detectable label, or may be conjugated to a toxin or a targeting moiety or enzyme (e.g. via a peptidyl bond or linker). For example, a targeted binding agent may comprise a catalytic site (e.g. in an enzyme domain) as well as an antigen binding site, wherein the antigen binding site binds to the antigen and thus targets the catalytic site to the antigen. The catalytic site may inhibit biological function of the antigen, e.g. by cleavage.

In connection with the generation of advanced antibody therapeutics, where complement fixation is a desirable attribute, it may be possible to sidestep the dependence on complement for cell killing through the use of bispecific antibodies, immunotoxins, or radiolabels, for example.

Antibodies can also be modified to act as immunotoxins, utilizing techniques that are well known in the art. See e.g., Vitetta Immunol Today 14:252 (1993). See also U.S. Pat. No. 5,194,594. In connection with the preparation of radiolabeled antibodies, such modified antibodies can also be readily prepared utilizing techniques that are well known in the art. See e.g., Junghans et al. in Cancer Chemotherapy and Biotherapy 655-686 (2d edition, Chafner and Longo, eds., Lippincott Raven (1996)). See also U.S. Pat. Nos. 4,681,581, 4,735,210, 5,101,827, 5,102,990 (RE 35,500), 5,648,471, and 5,697,902. Each immunotoxin or radiolabeled molecule would be likely to kill cells expressing the desired multimeric enzyme subunit oligomerisation domain.

When an antibody is linked to an agent (e.g., radioisotope, pharmaceutical composition, or a toxin), it is contemplated that the agent possess a pharmaceutical property selected from the group of antimitotic, alkylating, antimetabolite, antiangiogenic, apoptotic, alkaloid, COX-2, and antibiotic agents and combinations thereof. The drug can be selected from the group of nitrogen mustards, ethylenimine derivatives, alkyl sulfonates, nitrosoureas, triazenes, folic acid analogs, anthracyclines, taxanes, COX-2 inhibitors, pyrimidine analogs, purine analogs, antimetabolites, antibiotics, enzymes, epipodophyllotoxins, platinum coordination complexes, vinca alkaloids, substituted ureas, methyl hydrazine derivatives, adrenocortical suppressants, antagonists, endostatin, taxols, camptothecins, oxaliplatin, doxorubicins and their analogs, and a combination thereof.

Examples of toxins further include gelonin, Pseudomonas exotoxin (PE), PE40, PE38, diphtheria toxin, ricin, abrin, alpha toxin, saporin, ribonuclease (RNase), DNase I, Staphylococcal enterotoxin-A, pokeweed antiviral protein, gelonin, Pseudomonas endotoxin, members of the enediyne family of molecules, such as calicheamicin and esperamicin, as well as derivatives, combinations and modifications thereof. Chemical toxins can also be taken from the group consisting of duocarmycin (see, e.g., U.S. Pat. No. 5,703,080 and U.S. Pat. No. 4,923,990), methotrexate, doxorubicin, melphalan, chlorambucil, ARA-C, vindesine, mitomycin C, cis-platinum, etoposide, bleomycin and 5-fluorouracil. Examples of chemotherapeutic agents also include Adriamycin, Doxorubicin, 5-Fluorouracil, Cytosine arabinoside (Ara-C), Cyclophosphamide, Thiotepa, Taxotere (docetaxel), Busulfan, Cytoxin, Taxol, Methotrexate, Cisplatin, Melphalan, Vinblastine, Bleomycin, Etoposide, Ifosfamide, Mitomycin C, Mitoxantrone, Vincreistine, Vinorelbine, Carboplatin, Teniposide, Daunomycin, Caminomycin, Aminopterin, Dactinomycin, Mitomycins, Esperamicins (see, U.S. Pat. No. 4,675,187), Melphalan, and other related nitrogen mustards. Suitable toxins and chemotherapeutic agents are described in Remington's Pharmaceutical Sciences, 19th Ed. (Mack Publishing Co. 1995), and in Goodman And Gilman's The Pharmacological Basis of Therapeutics, 7th Ed. (MacMillan Publishing Co. 1985). Other suitable toxins and/or chemotherapeutic agents are known to those of skill in the art.

Examples of radioisotopes include gamma-emitters, positron-emitters, and x-ray emitters that can be used for localisation and/or therapy, and beta-emitters and alpha-emitters that can be used for therapy. The radioisotopes described previously as useful for diagnostics, prognostics and staging are also useful for therapeutics.

Non-limiting examples of anti-cancer or anti-leukemia agents include anthracyclines such as doxorubicin (adriamycin), daunorubicin (daunomycin), idarubicin, detorubicin, caminomycin, epirubicin, esorubicin, and morpholino and substituted derivatives, combinations and modifications thereof. Exemplary pharmaceutical agents include cis-platinum, taxol, calicheamicin, vincristine, cytarabine (Ara-C), cyclophosphamide, prednisone, daunorubicin, idarubicin, fludarabine, chlorambucil, interferon alpha, hydroxyurea, temozolomide, thalidomide, and bleomycin, and derivatives, combinations and modifications thereof. Preferably, the anti-cancer or anti-leukemia is doxorubicin, morpholinodoxorubicin, or morpholinodaunorubicin.

The antibodies of the invention also encompass antibodies that have half-lives (e.g., serum half-lives) in a mammal, preferably a human, of greater than that of an unmodified antibody. Said antibody half life may be greater than about 15 days, greater than about 20 days, greater than about 25 days, greater than about 30 days, greater than about 35 days, greater than about 40 days, greater than about 45 days, greater than about 2 months, greater than about 3 months, greater than about 4 months, or greater than about 5 months. The increased half-lives of the antibodies of the present invention or fragments thereof in a mammal, preferably a human, result in a higher serum titer of said antibodies or antibody fragments in the mammal, and thus, reduce the frequency of the administration of said antibodies or antibody fragments and/or reduces the concentration of said antibodies or antibody fragments to be administered. Antibodies or fragments thereof having increased in vivo half-lives can be generated by techniques known to those of skill in the art. For example, antibodies or fragments thereof with increased in vivo half-lives can be generated by modifying (e.g., substituting, deleting or adding) amino acid residues identified as involved in the interaction between the Fc domain and the FcRn receptor (see, e.g., International Publication Nos. WO 97/34631 and WO 02/060919, which are incorporated herein by reference in their entireties). Antibodies or fragments thereof with increased in vivo half-lives can be generated by attaching to said antibodies or antibody fragments polymer molecules such as high molecular weight polyethyleneglycol (PEG). PEG can be attached to said antibodies or antibody fragments with or without a multifunctional linker either through site-specific conjugation of the PEG to the N- or C-terminus of said antibodies or antibody fragments or via epsilon-amino groups present on lysine residues. Linear or branched polymer derivatisation that results in minimal loss of biological activity will be used. The degree of conjugation will be closely monitored by SDS-PAGE and mass spectrometry to ensure proper conjugation of PEG molecules to the antibodies. Unreacted PEG can be separated from antibody-PEG conjugates by, e.g., size exclusion or ion-exchange chromatography.

As will be appreciated by one of skill in the art, in the above embodiments, while affinity values can be important, other factors can be as important or more so, depending upon the particular function of the antibody. For example, for an immunotoxin (toxin associated with an antibody), the act of binding of the antibody to the target can be useful; however, in some embodiments, it is the internalisation of the toxin into the cell that is the desired end result. As such, antibodies with a high percent internalisation can be desirable in these situations. Thus, in one embodiment, antibodies with a high efficiency in internalisation are contemplated. A high efficiency of internalisation can be measured as a percent internalised antibody, and can be from a low value to 100%. For example, in varying embodiments, 0.1-5, 5-10, 10-20, 20-30, 30-40, 40-45, 45-50, 50-60, 60-70, 70-80, 80-90, 90-99, and 99-100% can be a high efficiency. As will be appreciated by one of skill in the art, the desirable efficiency can be different in different embodiments, depending upon, for example, the associated agent, the amount of antibody that can be administered to an area, the side effects of the antibody-agent complex, the type (e.g., cancer type) and severity of the problem to be treated.

In other embodiments, the antibodies disclosed herein provide an assay kit for the detection of α5β1 expression in mammalian tissues or cells in order to screen for a disease or disorder associated with changes in expression of α5β1. The kit comprises an antibody that binds α5β1 and means for indicating the reaction of the antibody with the antigen, if present.

### Combinations

The targeted binding agent or antibody defined herein may be applied as a sole therapy or may involve, in addition to the compounds of the invention, conventional surgery or radiotherapy or chemotherapy. Such chemotherapy may include one or more of the following categories of anti tumour agents:

(i) other antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);

(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;

(iii) anti-invasion agents (for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341) and N-(2-chloro-6-methylphenyl)-2-{6-[4-(2-hydro xyethyl)piperazin-1-yl]-2-methylpyrimidin-4-ylamino}thiazole-5-carboxamide (dasatinib, BMS-354825; J. Med. Chem., 2004, 47, 6658-6661), and metalloproteinase inhibitors like marimastat, inhibitors of urokinase plasminogen activator receptor function or, inhibitors of cathepsins, inhibitors of serine proteases for example matriptase, hepsin, urokinase, inhibitors of heparanase);

(iv) cytotoxic agents such as fludarabine, 2-chlorodeoxyadenosine, chlorambucil or doxorubicin and combination thereof such as Fludarabine+cyclophosphamide, CVP: cyclophosphamide+vincristine+prednisone, ACVBP: doxorubicin+cyclophosphamide+vindesine+bleomycin+prednisone, CHOP: cyclophosphamide+doxorubicin+vincristine+prednisone, CNOP: cyclophosphamide+mitoxantrone+vincristine+prednisone, m-BACOD: methotrexate+bleomycin+doxorubicin+cyclophosphamide+vincristine+dexamethasone+leucovorin., MACOP-B: methotrexate+doxorubicin+cyclophosphamide+vincristine+prednisone fixed dose+bleomycin+leucovorin, or ProMACECytaBOM: prednisone+doxorubicin+cyclophosphamide+etoposide+cytarabine+bleomycin+vincristine+methotrexate+leucovorin.

(v) inhibitors of growth factor function, for example such inhibitors include growth factor antibodies and growth factor receptor antibodies (for example the anti-erbB2 antibody trastuzumab [Herceptin™], the anti-EGFR antibody panitumumab, the anti-erbB1 antibody cetuximab [Erbitux, C225] and any growth factor or growth factor receptor antibodies disclosed by Stern et al. Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI-1033), erbB2 tyrosine kinase inhibitors such as lapatinib, inhibitors of the hepatocyte growth factor family, inhibitors of the platelet-derived growth factor family such as imatinib, inhibitors of serine/threonine kinases (for example Ras/Raf signalling inhibitors such as farnesyl transferase inhibitors, for example sorafenib (BAY 43-9006)), inhibitors of cell signalling through MEK and/or AKT kinases, inhibitors of the hepatocyte growth factor family, c-kit inhibitors, abl kinase inhibitors, IGF receptor (insulin-like growth factor) kinase inhibitors, aurora kinase inhibitors (for example AZD1152, PH739358, VX-680, MLN8054, R763, MP235, MP529, VX-528 and AX39459), cyclin dependent kinase inhibitors such as CDK2 and/or CDK4 inhibitors, and inhibitors of survival signaling proteins such as Bcl-2, Bcl-XL for example ABT-737;

(vi) antiangiogenic agents such as those which inhibit the effects of vascular endothelial growth factor, [for example the anti-vascular endothelial cell growth factor antibody bevacizumab (Avastin™) and VEGF receptor tyrosine kinase inhibitors such as 4-(4-bromo-2-fluoroanilino)-6-methoxy-7-(1-methylpiperidin-4-ylmethoxy)quinazoline (ZD6474; Example 2 within WO 01/32651), 4-(4-fluoro-2-methylindol-5-yloxy)-6-methoxy-7-(3-pyrrolidin-1-ylpropoxy)quinazoline (AZD2171; Example 240 within WO 00/47212), vatalanib (PTK787; WO 98/35985) and SU11248 (sunitinib; WO 01/60814), compounds such as those disclosed in International Patent Applications WO 97/22596, WO 97/30035, WO 97/32856, WO 98/13354, WO 00/47212 and WO 01/32651 and compounds that work by other mechanisms (for example linomide, inhibitors of integrin αvβ3 function and angiostatin)] or colony stimulating factor 1 (CSF1) or CSF1 receptor;

(vii) vascular damaging agents such as Combretastatin A4 and compounds disclosed in International Patent Applications WO 99/02166, WO 00/40529, WO 00/41669, WO 01/92224, WO 02/04434 and WO 02/08213;

(viii) antisense therapies, for example those which are directed to the targets listed above, such as G-3139 (Genasense), an anti bcl2 antisense;

(ix) gene therapy approaches, including for example approaches to replace aberrant genes such as aberrant p53 or aberrant BRCA1 or BRCA2, GDEPT (gene directed enzyme pro drug therapy) approaches such as those using cytosine deaminase, thymidine kinase or a bacterial nitroreductase enzyme and approaches to increase patient tolerance to chemotherapy or radiotherapy such as multi drug resistance gene therapy; and

(x) immunotherapy approaches, including for example treatment with Alemtuzumab (campath-1H™), a monoclonal antibody directed at CD52, or treatment with antibodies directed at CD22, ex vivo and in vivo approaches to increase the immunogenicity of patient tumour cells, transfection with cytokines such as interleukin 2, interleukin 4 or granulocyte macrophage colony stimulating factor, approaches to decrease T cell anergy such as treatment with monoclonal antibodies inhibiting CTLA-4 function, approaches using transfected immune cells such as cytokine transfected dendritic cells, approaches using cytokine transfected tumour cell lines and approaches using anti idiotypic antibodies.

(xi) inhibitors of protein degradation such as proteasome inhibitor such as Velcade (bortezomid).

(xii) biotherapeutic therapeutic approaches for example those which use peptides or proteins (such as antibodies or soluble external receptor domain constructions) which either sequester receptor ligands, block ligand binding to receptor or decrease receptor signalling (e.g. due to enhanced receptor degradation or lowered expression levels).

In one embodiment the anti-tumour treatment defined herein may involve, in addition to the compounds of the invention, treatment with other antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin).

In one embodiment the anti-tumour treatment defined herein may involve, in addition to the compounds of the invention, treatment with gemcitabine.

Such conjoint treatment may be achieved by way of the simultaneous, sequential or separate dosing of the individual components of the treatment. Such combination products employ the compounds of this invention, or pharmaceutically acceptable salts thereof, within the dosage range described hereinbefore and the other pharmaceutically active agent within its approved dosage range.

For treatment of an inflammatory disease, e.g. rheumatoid arthritis, osteoarthritis, asthma, allergic thinitis, chronic obstructive pulmonary disease (COPD), or psoriasis, a targeted binding agent of the invention may be combined with one or agents, such as non-steroidal anti-inflammatory agents (hereinafter NSAIDs) including non-selective cyclo-oxygenase (COX)-1/COX-2 inhibitors whether applied topically or systemically, such as piroxicam, diclofenac, propionic acids, such as naproxen, flurbiprofen, fenoprofen, ketoprofen and ibuprofen, fenamates, such as mefenamic acid, indomethacin, sulindac, azapropazone, pyrazolones, such as phenylbutazone, salicylates, such as aspirin); selective COX-2 inhibitors (such as meloxicam, celecoxib, rofecoxib, valdecoxib, lumarocoxib, parecoxib and etoricoxib); cyclo-oxygenase inhibiting nitric oxide donors (CINODs); glucocorticosteroids (whether administered by topical, oral, intra-muscular, intra-venous or intra-articular routes); methotrexate, leflunomide; hydroxychloroquine, d-penicillamine, auranofin or other parenteral or oral gold preparations; analgesics; diacerein; intra-articular therapies, such as hylauronic acid derivatives; and nutritional supplements, such as glucosamine

The targeted binding agent or antibody defined herein may be applied in combination with an antagonist of VEGF. The targeted binding agent or antibody defined herein and the antagonist of VEGF can be administered in concurrent or sequential treatment cycles. Such combination treatments are useful for treating diseases having abnormal angiogenesis and/or vascular permeability. In one embodiment, the antagonist of VEGF is Avastin™, ZD6474 (4-(4-bromo-2-fluoroanilino)-6-methoxy-7-(1-methylpiperidin-4-ylmethoxy)quinazoline) or AZD2171 (4-(4-fluoro-2-methylindol-5-yloxy)-6-methoxy-7-(3-(pyrrolidin-1-yl)propoxy)quinazoline).

All references cited herein, including patents, patent applications, papers, text books, and the like, and the references cited therein, to the extent that they are not already, are hereby incorporated herein by reference in their entirety.

This application claims the benefit of priority of U.S. Provisional Application Ser. No. 61/140,331 filed Dec. 23, 2008, herein incorporated by reference for all purposes.

### EXAMPLES

The following examples, including the experiments conducted and results achieved are provided for illustrative purposes only and are not to be construed as limiting upon the teachings herein.

### Example 1

### Immunization and Titering

### Immunization

Immunizations were conducted using soluble α5β1 and cell-bound α5β1 (CHO transfectants expressing human α531 at the cell surface), respectively. For the generation of CHO transfectants, human full-length α5β1 cDNA was inserted into the pcDNA 3 expression vector. CHO cells were transiently transfected via electroporation. Expression of human α5 μl on the cell surface at the level suitable for immunogen purpose was confirmed by Fluorescence-Activated Cell Sorter (FACS) analysis. For the campaign, an initial injection of 2×10⁶ cells/mouse of transfected CHO cells (group 1 and 2) and 10 μg/mouse of soluble protein (Groups 3 and 4) were used for immunization in XenoMouse™. The immunization was carried out according to the methods disclosed in U.S. patent application Ser. No. 08/759,620, filed Dec. 3, 1996 and International Patent Application Nos. WO 98/24893, published Jun. 11, 1998 and WO 00/76310, published Dec. 21, 2000, the disclosures of which are hereby incorporated by reference. Following the initial immunization, 11 subsequent boost immunizations of 1×10⁶ cells/mouse were administered for groups 1 and 2 (cell-bound antigen), and thirteen subsequent boost immunizations of 5 μg/mouse were administered for groups 3 and 4 (soluble antigen). The immunization programs are summarized in Table 2.

### Selection of Animals for Harvest by Titer

Titers of the antibody against native (cell-bound) antigen were tested by FACS staining for native antigen binding using untransfected 300.19 cells (Amgen, Vancouver) or human α5β1-transfected 300.19 cells. At the end of the immunization program, fusions were performed using mouse myeloma cells and lymphocytes isolated from the spleens and lymph nodes of the immunized mice by means of electroporation, as described in Example 2.

TABLE 2
Summary of Immunization Programs

| | | | No of | |
|---|---|---|---|---|
| Group | Immunogen | Strain | mice | Immunization routes |
| 1 | Cell-bound | IgG2 | 10 | IP/Tail/BIP, twice/wk, × |
| | α5β1 (CHO | | | 5 wks, |
| | transfectants) | | | |
| 2 | Cell-bound | IgG4 | 10 | IP/Tail/BIP, twice/wk, × |
| | α5β1 (CHO | | | 5 wks |
| | transfectants) | | | |
| 3 | Soluble α5β1 | IgG2 | 10 | IP/Tail/BIP, twice/wk, × |
| | | | | 6 wks |
| 4 | Soluble α5β1 | IgG4 | 10 | IP/Tail/BIP, twice/wk, × |
| | | | | 6 wks |

“IP” refers to “intraperitoneal”
“BIP” refers to “Base of Tail/Intraperitoneal”

### Example 2

### Recovery of Lymphocytes, B-Cell Isolations, Fusions and Generation Of Hybridomas

Immunized mice were sacrificed by cervical dislocation, and the draining lymph nodes harvested and pooled from each cohort. Three independent harvests were conducted. Harvest 1 used five mice from group 1 with ID numbers 150927, 150928, 150929, 150930, 150031. Harvest 2 used six mice from group 2 with ID numbers 151037, 151038, 151039, 151040, 150588, 150589. The third harvest used five mice from group 1 with ID numbers 150932, 150919, 150920, 150921, 150926.

The lymphoid cells were dissociated by grinding in DMEM to release the cells from the tissues and the cells were suspended in DMEM. The cells were counted, and 0.9 ml DMEM per 100 million lymphocytes added to the cell pellet to resuspend the cells gently but completely. Using 100 μl of CD90+ magnetic beads per 100 million cells, the cells were labeled by incubating the cells with the magnetic beads at 4° C. for 15 minutes. The magnetically labeled cell suspension containing up to 10⁸ positive cells (or up to 2×10⁹ total cells) was loaded onto a LS+ column and the column washed with DMEM. The total effluent was collected as the CD90 negative fraction (most of these cells were expected to be B cells).

The fusion was performed by mixing washed enriched Day 6 B cells with nonsecretory myeloma P3X63Ag8.653 cells purchased from ATCC, cat.# CRL 1580 (Kearney et al, J. Immunol. 123, 1979, 1548-1550) at a ratio of 1:4. The cell mixture was gently pelleted by centrifugation at 400×g for 4 minutes. After decanting of the supernatant, the cells were gently mixed using a 1 ml pipette. Preheated PEG (1 ml per 10⁶ B-cells) was slowly added with gentle agitation over 1 minute followed by 1 minute of mixing. Preheated IDMEM (2 ml per 10⁶ B-cells) was then added over 2 minutes with gentle agitation. Finally preheated IDMEM (8 ml per 10⁶ B-cells) was added over 3 minutes.

The fused cells were spun down at 400×g for 6 minutes and resuspended in 20 ml of Selection media (DMEM (Invitrogen), 15% FBS (Hyclone), supplemented with L-glutamine, pen/strep, MEM Non-essential amino acids, Sodium Pyruvate, 2-Mercaptoethanol (all from Invitrogen), HA-Azaserine Hypoxanthine and OPI (oxaloacetate, pyruvate, bovine insulin) (both from Sigma) and IL-6 (Boehringer Mannheim)) per 10⁶ B-cells. Cells were incubated for 20-30 minutes at 37° C. and then resuspended in 200 ml Selection media and cultured for 3-4 days in a T175 flask.

Day 3 post fusion the cells were collected, spun for 8 minutes at 400×g and resuspended in 10 ml Selection media per 10⁶ fused B-cells. FACS analysis of hybridoma population was performed, and cells were subsequently frozen down.

Hybridomas were grown as routine in the selective medium. Exhaustive supernatants collected from the hybridomas that potentially produce anti-human α5β1 antibodies were subjected to subsequent screening assays.

### Example 3

### Antibody Titer Measurement: Native Antigen Binding of 300.19 Cells

FACS analysis was performed on 300.19 cells (Amgen, Vancouver) to measure the titers of antibody against α5β1 expressed on B300.19 cells. 300.19 cells (control and transfected with human α5β1) were seeded at 50,000 cells/well and incubated with 90 μL of sample supernatant (at 1:50 dilution) for one hour at 4° C. The wells were then washed and incubated with Cy5-conjugated goat anti-human antibody (Jackson Laboratories) at 5 μg/mL and 7-Amino-Actinomycin (7AAD) at 5 μg/mL for 15 minutes at 4° C. Bound α5β1 was detected using FACS analysis. The positive control was mouse anti-α5β1 antibody (R&D Systems, Inc.), and negative controls included goat anti-human and anti-mouse Fc Cy5 coupled antibody (Jackson Laboratories) alone, as well binding or irrelevant mouse IgG1 and human IgG2, irrelevant supernatants as indicated. Animals with the greatest FACS Geometric Mean Fluorescence were selected for subsequent hybridoma generation. Table 3 lists the FACS data obtained from analysis of the 300.19 cells.

TABLE 3
Titers of antibody against human α5β1 as measured by
FACS analysis of 300.19 cells

| | | | | α5β1 expressing | | | |
|---|---|---|---|---|---|---|---|
| | | | | B300.19 cells | | | |
| | B300.19 cells | | | | | X | |
| | | % | X Geo | | % | Geo | |
| ID's | Events | Total | Mean | Events | Total | Mean | Ratio |
| cells | 1397 | 13.97 | 3.0 | 2795 | 28 | 4.2 | 1.4 |
| Cells + Mo 2′ | 1777 | 17.77 | 18.1 | 1548 | 15 | 15.4 | 0.9 |
| alone | | | | | | | |
| Cells + Hu 2′ | 1730 | 17.3 | 15.1 | 1450 | 15 | 14.0 | 0.9 |
| alone | | | | | | | |
| Mo IgG1 | 1453 | 14.53 | 17.3 | 1229 | 12 | 14.9 | 0.9 |
| 2.0 ug/mL + 2′ | | | | | | | |
| Hu IgG2 @ | 1372 | 13.72 | 14.5 | 1144 | 11 | 12.0 | 0.8 |
| 2.0 ug/mL + 2′ | | | | | | | |
| Irrel. Sera1 | 1907 | 19.07 | 25.1 | 1494 | 15 | 20.3 | 0.8 |
| Irrel. Sera2 | 2041 | 20.41 | 44.4 | 1761 | 18 | 24.5 | 0.6 |
| 150919 | 1912 | 19.12 | 23.8 | 1505 | 15 | 95.6 | 4.0 |
| 150920 | 2185 | 21.85 | 25.1 | 1669 | 17 | 92.6 | 3.7 |
| 150921 | 2012 | 20.12 | 69.4 | 1636 | 16 | 142.5 | 2.1 |
| 150926 | 2031 | 20.31 | 23.7 | 1627 | 16 | 77.0 | 3.3 |
| 150927 | 1535 | 15.35 | 35.1 | 1340 | 13 | 156.2 | 4.5 |
| 150928 | 2003 | 20.03 | 28.4 | 1760 | 18 | 251.6 | 8.9 |
| 150929 | 1862 | 18.62 | 22.4 | 1614 | 16 | 102.0 | 4.5 |
| 150930 | 2118 | 21.18 | 23.9 | 1852 | 19 | 154.1 | 6.4 |
| 150931 | 2006 | 20.06 | 20.3 | 1659 | 17 | 117.5 | 5.8 |
| 150932 | 2228 | 22.28 | 30.3 | 1796 | 18 | 107.2 | 3.5 |
| 151037 | 1978 | 19.78 | 18.5 | 1680 | 17 | 77.1 | 4.2 |
| 151038 | 2218 | 22.18 | 22.6 | 1882 | 19 | 27.0 | 1.2 |
| 151039 | 2099 | 20.99 | 29.5 | 1698 | 17 | 88.7 | 3.0 |
| 151040 | 2013 | 20.13 | 24.4 | 1627 | 16 | 75.6 | 3.1 |
| 150588 | 2323 | 23.23 | 21.1 | 1865 | 19 | 81.6 | 3.9 |
| 150589 | 2448 | 24.48 | 17.1 | 2016 | 20 | 62.1 | 3.6 |
| mAb 1969 @ | 2048 | 20.48 | 20.4 | 1804 | 18 | 194.1 | 9.5 |
| 2.0 ug/mL | | | | | | | |
| mAb 1969 @ | 2062 | 20.62 | 22.1 | 1796 | 18 | 151.8 | 6.9 |
| 0.2 ug/mL | | | | | | | |
| mAb 1969 @ | 2109 | 21.09 | 18.1 | 1769 | 18 | 45.1 | 2.5 |
| 0.02 ug/mL | | | | | | | |

Only fusions derived from animals receiving cell bound immunogen were progressed to further screening.

### Example 4

### Hybridoma Supernatant Screening by Binding Assay-Binding to α5β1 Expressed on HEK 293T Cells

Hybridoma supernatants containing antibody, produced as described in Examples 1 and 2, were screened by assays that measure binding to immobilized native α5β1. Supernatants collected from harvested cells were tested to assess the binding of secreted antibodies to HEK 293T (ATCC, cat.# CRL 11268) cells. Cells in FACS buffer were seeded into 384-well FMAT plates in a volume of 40 μL/well at a density of 7500 cells/well. Then, 10 μL/well of supernatant was added, and plates were incubated for approximately 1.5 hour at room temperature, after which 10 μL/well of anti-human IgG-Cy5 secondary antibody (Jackson Laboratories) was added to a final concentration of 750 ng/ml. Plates were then incubated for one hour at 4° C., and fluorescence was read using an FMAT macroconfocal scanner (Applied Biosystems). A total of 1790 antigen-specific wells were identified across the three harvests. The breakdown of these hits were as follows; harvest 1—459 native binding wells identified, harvest 2—860 native binding wells identified and harvest 3—471 native binding wells identified.

### Example 5

### Determination of Relative Potency of Antibody-Containing Supernatants: Ability to Inhibit α5β1 Mediated Binding TO of Fibronectin

The relative potency of the different antibody-containing supernatants was assayed by how well the antibodies blocked adhesion of K562 cells (ATCC, cat.# CCL 243) to fibronectin. Plates were coated overnight with 3-5 μg/ml Fibronectin or GST-Fibronectin type III domains 9-10, and pre-blocked with 3% BSA/PBS for 1 hour prior to the assay. Cells were then pelleted and washed twice in HBSS, after which the cells were then resuspended in HBSS at 1×10⁶ cells/ml. To select the best antibodies the cells were incubated in the presence of appropriate antibodies at 4° C. for 60 minutes in a V-bottom plate. To increase the stringency of the assay cells the pre-incubation step was removed, and the cation conditions modified to increase binding affinity. The 3% BSA/PBS was removed from the assay plates and the plates washed twice with PBS or HBSS, and the cell-antibody mixtures were transferred to the coated plate and the plate was incubated at 37° C. for 60 minutes in the presence of either 1 mM or 0.2 mM MnCl₂. The cells on the coated plates were then washed four times in warm HBSS, and the cells were thereafter frozen at −80° C. for one hour. The cells were allowed to thaw at room temperature for one hour, and then 100 μL, of CyQuant dye/lysis buffer (Molecular Probes) was added to each well according to the manufacturer's instructions. Fluorescence was read at an excitation wavelength of 485 nm and an emission wavelength of 530 nm.

To identify anti-functional antibodies two adhesion assays (n=1 and n=2 in Table 3) were run using supernatant diluted 1 in 4 and with pre-incubation of the antibodies on cells prior to addition to plates coated with full length FN and MnCl₂ was included at 1 mM final. Many neutralizing antibodies were identified and a cut-off of 90% inhibition of adhesion was applied for these screens. A total of 188 supernatants were advanced for further screening based on this data (Harvest 1—16 Abs; Harvest 2—116 Abs; Harvest 3−56 Abs). To identify the best antibodies within this group the K562 adhesion assay was run a third time (n=3 Table 3) without pre-incubation on cells to attempt to identify antibodies with higher affinity (or good on-rates). The supernatant was still used at 1 in 4 dilution. The antibodies and cells (in media containing 0.2 mM MnCl₂) were added to a plates coated with GST-FNIII(9-10) at 3 ug/mL overnight. Most of the antibodies still showed nearly complete inhibition in this assay. In an attempt to increase the assay stringency, two more adhesion assays (n=4 and N=5 Table 3) were run with K562 cells on GST-FNIII(9-10) (in media containing 1 mM MnCl₂) with a larger dilution of supernatant (1 in 10). The antibodies were found to have differences in their ability to block adhesion at this dilution, and allowed the selection of a lead panel of antibodies for sub-cloning. Table 4 provides a summary of the results for the assay.

TABLE 4
Inhibition of adhesion of K562 cell binding to fibronectin by selected lead
antibodies

| | | % Inhibition of cell adhesion | | | | |
|---|---|---|---|---|---|---|
| MAb ID | Isotype | n = 1 | n = 2 | n = 3 | n = 4 | n = 5 |
| 2C7 | IgG2/k | 98 | 95 | 90 | 98 | 95 |
| 5B11 | IgG2/k | 96 | 98 | 90 | 97 | 102 |
| 5A7 | IgG2/k | 96 | 91 | 90 | 97 | 100 |
| 3C5 | IgG2/k | 97 | 96 | 99 | 97 | 104 |
| 2C5.2B12 | IgG2/k | 100 | 96 | 101 | 96 | 97 |
| 7D11 | IgG4/k | 98 | 101 | 100 | 96 | 93 |
| 5F12 | IgG4/k | 97 | 101 | 96 | 95 | 82 |
| 5A10 | IgG2/k | 98 | 96 | 96 | 95 | 101 |
| 9E2.3A8 | IgG4/k | 98 | 100 | 94 | 94 | 87 |
| 2A9.1A1 | IgG2/k | 100 | 94 | 96 | 94 | 100 |
| 3C2.2A8 | IgG2/k | 99 | 100 | 102 | 94 | 96 |
| 4F1 | IgG2/k | 99 | 100 | 98 | 94 | 95 |
| 4E9 | IgG4/k | 96 | 99 | 94 | 93 | 85 |
| 5B1 | IgG4/k | 95 | 100 | 77 | 93 | 81 |
| 3G11.1A6 | IgG2/k | 99 | 99 | 93 | 92 | 100 |
| 4E4 | IgG2/k | 99 | 100 | 97 | 92 | 88 |
| 8A6.1A3 | IgG4/k | 99 | 95 | 91 | 92 | 90 |
| 3C11 | IgG2/k | 99 | 97 | 97 | 92 | 98 |
| 2F5.1A4 | IgG2/k | 96 | 100 | 103 | 91 | 84 |
| 7G2 | IgG4/k | 98 | 101 | 99 | 89 | 88 |
| 8F3 | IgG4/k | 93 | 97 | 101 | 88 | 75 |
| 7H8 | IgG4/k | 96 | 101 | 83 | 87 | 80 |
| 2E10.1B9 | IgG2/k | 97 | 94 | 96 | 86 | 87 |
| 6H5 | IgG4/k | 96 | 100 | 95 | 86 | 76 |
| 2F1 | IgG2/k | 96 | 96 | 90 | 85 | 64 |
| 7B2.3B10 | IgG4/k | 93 | 100 | 94 | 85 | 75 |
| 2H7 | IgG2/k | 100 | 97 | 42 | 83 | 81 |
| 7F6 | IgG4/k | 95 | 101 | 79 | 82 | 81 |
| 8B2 | IgG4/k | 97 | 108 | 98 | 80 | 78 |

### Example 6

### Screening for Functional Selectivity Over α4β1 Integrin—Antibodies Do Not Inhibit J6 Cell Adhesion to the Cs-1 Fragment of Fibronectin

To confirm that the antibodies were specific to α5 or α5β1 (and not binding to β1), the antibodies were also screened in an alpha4beta dependent adhesion assay. For this, the ability of our antibodies to block the binding of J6.77 Jurkat cells (Amgen, Vancouver) to the CS-1 fragment of fibronectin was tested. Plates were coated overnight at 4° C. with 2.5 ug/ml GST-CS-1 fragment of fibronectin in PBS, washed twice in PBS and then blocked with 3% BSA/PBS for 1 hour. Cells were then pelleted and washed 3 times with 1% BSA/HBSS and resuspended in HBSS at a concentration of 9×10⁵/ml. Cells were dispensed into V bottom pates (37.5 ul per well), 12.5 ul of supernatant or a control of HBSS added to each well, and then incubated for 1 hour at 4° C. Assay plates were then washed 3 times with PBS. The mix of cells and antibody was then transferred to the assay plate and incubated for 40 minutes at 37° C. in the presence of 0.2 mM MnCl₂. The cells on the coated plates were then washed four times in warm HBSS, and the cells were thereafter frozen at −80° C. for one hour. The cells were allowed to thaw at room temperature for one hour, and then 100 μL of CyQuant dye/lysis buffer (Molecular Probes) was added to each well according to the manufacturer's instructions. Fluorescence was read at an excitation wavelength of 485 nm and an emission wavelength of 530 nm. The majority of the antibodies showed little to no blockade in this assay, suggesting that their specificity is primarily against α5 or α5β1 (Table 5). Table 5 provides a summary of the results for the assay.

TABLE 5
Inhibition of α4β1 mediated adhesion of J6.77 Jurkat cells
to the CS-1 fragment of fibronectin

% Inhibition
of adhesion

| MAb ID | Isotype | CS1 n = 1 | CS1 n = 2 |
|---|---|---|---|
| 2C7 | IgG2/k | 40 | 8 |
| 5B11 | IgG2/k | 27 | −15 |
| 5A7 | IgG2/k | 7 | 17 |
| 3C5 | IgG2/k | 24 | −4 |
| 2C5.2B12 | IgG2/k | 14 | −5 |
| 7D11 | IgG4/k | 8 | −2 |
| 5F12 | IgG4/k | 32 | 17 |
| 5A10 | IgG2/k | 45 | 32 |
| 9E2.3A8 | IgG4/k | 40 | 10 |
| 2A9.1A1 | IgG2/k | 2 | 11 |
| 3C2.2A8 | IgG2/k | 22 | 18 |
| 4F1 | IgG2/k | 19 | 29 |
| 4E9 | IgG4/k | 17 | 24 |
| 5B1 | IgG4/k | 54 | 37 |
| 3G11.1A6 | IgG2/k | 17 | 9 |
| 4E4 | IgG2/k | 39 | 26 |
| 8A6.1A3 | IgG4/k | 24 | 9 |
| 3C11 | IgG2/k | 15 | −43 |
| 2F5.1A4 | IgG2/k | 31 | −8 |
| 7G2 | IgG4/k | −18 | −7 |
| 8F3 | IgG4/k | 6 | 16 |
| 7H8 | IgG4/k | 58 | 18 |
| 2E10.1B9 | IgG2/k | 31 | −5 |
| 6H5 | IgG4/k | 22 | 16 |
| 2F1 | IgG2/k | 30 | 12 |
| 7B2.3B10 | IgG4/k | 45 | 21 |
| 2H7 | IgG2/k | 14 | 14 |
| 7F6 | IgG4/k | 19 | 4 |
| 8B2 | IgG4/k | 34 | 7 |

### Example 7

### Specific Binding to α5β1—Lead Antibodies Show No Cross Reactivity to the A5 Null Line HT29 when analysed by FACS

To confirm the antibodies bind to the α5 chain or to the α5β1 heterodimer, binding to Human colon adenocarcinoma grade II cells (HT29 cells) was assessed by FACS. HT29 (ATCC, cat #HTB-38) cells do not express the α5 chain, but do express the 131 chain. HT-29 cells were suspended in HBSS with 1% BSA and 1 mM final MnCL₂ at a concentration of 6×10⁵ cells/ml.12.5 uL of the primary antibody was added to 37.5 ul of cells and the plate incubated on ice for 60 minutes. A range of negative controls were included as described previously (indicated as below). In addition as a positive control antibodies to αvβ6 (Mab2077, Chemicon), α5β1 (Mab1909, Chemicon) and av (L230, Chemicon) were included. 100 uL of HBSS buffer was added to dilute primary antibody, the cells pelleted by centrifuging at 1500 rpm for 3 minutes and resuspended in 50 ul Goat anti-human IgG Fc Cy5 or Goat anti Mouse IgG Fc Cy5 secondary at 2 ug/mL. They were then incubated on ice for a further 7 minutes, and 100 uL of HBSS buffer was added to dilute secondary antibody. Finally cells were pelleted by centrifuging at 1500 rpm for 3 minutes, HBSS Buffer/secondary supernatant removed, washed twice in 100 ul of FACS buffer was added and the cells were resuspended and then read on a FACS Calibur HTS. Samples were analyzed using Cell Quest Pro software. The data is represented in Table 6 and confirm that the antibodies are selective for α5β1.

TABLE 6
Binding of lead antibodies to HT29 cells by FACS

| MAb ID | Binding Geomean |
|---|---|
| 7G2 | 3.2 |
| 7D11 | 2.92 |
| 8F3 | 3 |
| 2F5.1A4 | 2.94 |
| 7F6 | 3.2 |
| 5F12 | 2.92 |
| 6H5 | 3.15 |
| 9E2.3A8 | 2.86 |
| 4E4 | 3.51 |
| 7H8 | 3.03 |
| 8A6.1A3 | 2.99 |
| 7B2.3B10 | 3.1 |
| 8B2 | 4.19 |
| 2F1 | 3.02 |
| 2A9.1A1 | 4.73 |
| 2E10.1B9 | 3.11 |
| 5B1 | 2.9 |
| 2C5.2B12 | 3.6 |
| 3C2.2A8 | 3.29 |
| 2C7 | 4.9 |
| 2H7 | 29.26 |
| 5A7 | 13.13 |
| 3C5 | 4.04 |
| 5A10 | 26.55 |
| 5B11 | 4.62 |
| 3C11 | 11.41 |
| 3G11.1A6 | 3.02 |
| 4F1 | 3.45 |
| Cells Alone | 2.07 |
| Gt anti Mouse | 2.52 |
| Mouse IgG2a | 10.21 |
| Mouse IgG1 | 2.55 |
| MAB2077Z avb6 | 39.23 |
| MAB1969 a5b1 | 2.69 |
| 4b4 anti beta1 | 361.61 |
| L230 anti alphaV | 294.02 |
| Human Secondary | 2.8 |
| Human IgG2 | 2.84 |
| Human IgG4 | 3.2 |

### Example 8

### Lead Antibodies Show No Cross-Reactivity to Macaque α5β1

To determine whether the antibodies cross reacted to monkey integrin, binding to purified Macaque T-cells was assessed. Macaque PBMCs were previously purified from whole blood, and stored frozen. Macaque PBMCs were suspended in adhesion buffer with HBSS 1% BSA and 1 mM Mn2+ (“FACS buffer”), at a concentration of 4.8×10⁵ live cells per ml. 12.5 uL of the primary antibody was added to 37.5 ul of cells, and incubated at 4° C. for 1 hour. Positive and negative controls were included as indicated. 100 uL of FACs buffer is added to dilute out primary antibody, the cells washed and resuspended in the appropriate secondary at 2 ug/mL (50 uL) with 10 ug/mL 7AAD, and stained on ice for 7 minutes. 100 uL of FACs buffer was added and cells were washed twice in FACS buffer, finally the supernatant removed and the cells were resuspended in 100 uL of buffer.

Samples were read on HTS FACS machine and analyzed using Cell Quest Pro software. The data shown in Table 7 confirm that the antibodies cross react with monkey α5β1.

TABLE 7
Cross-Reactivity Assay Results Against Macaque α5β1

| | Geo Mean on Macaque |
|---|---|
| MAb ID | T-cells IgG detection |
| 2C7 | 11 |
| 5B11 | 12 |
| 5A7 | 17 |
| 3C5 | 16 |
| 2C5.2B12 | 11 |
| 7D11 | 15 |
| 5F12 | 12 |
| 5A10 | 16 |
| 9E2.3A8 | 12 |
| 2A9.1A1 | 12 |
| 3C2.2A8 | 10 |
| 4F1 | 13 |
| 4E9 | 9 |
| 5B1 | 9 |
| 3G11.1A6 | 13 |
| 4E4 | 14 |
| 8A6.1A3 | 11 |
| 3C11 | 12 |
| 2F5.1A4 | 13 |
| 7G2 | 12 |
| 8F3 | 12 |
| 7H8 | 13 |
| 2E10.1B9 | 12 |
| 6H5 | 13 |
| 2F1 | 12 |
| 7B2.3B10 | 12 |
| 7H11 | 12 |
| 2H7 | 10 |
| 7F6 | 12 |
| 8B2 | 11 |
| Cells alone | 2 |
| Goat anti Mouse | 2 |
| IgGFc Cy5 | |
| Mouse IgG1 | 2 |
| 4b4 anti beta1 | 141 |
| IIA1 anti alpha5 | 10 |
| Goat anti Human IgG | 3 |
| Fc Cy5 | |
| Human IgG2/4 | 3 |
| L230 anti-alphaV | 3 |

### Example 9

### Structural Analysis of α5β1 Antibodies

The variable heavy chains and the variable light chains of the antibodies were sequenced to determine their DNA sequences. The complete sequence information for the anti-α5β1 antibodies is provided in the sequence listing with nucleotide and amino acid sequences for each gamma and kappa chain combination. The heavy and light chain variable domain cDNA sequences were analyzed to determine the VH, D, JH, Vk and Jk gene segments used. The sequences were then translated to determine the primary amino acid sequence and compared to the germline VH-D-J H- or Vk-Jk sequences to assess mutations of lead antibody sequences from germ line.

Table 12 is a table comparing the antibody heavy chain regions to their cognate germ line heavy chain region. Table 13 is a table comparing the antibody kappa light chain regions to their cognate germ line light chain region.

The variable (V) regions of immunoglobulin chains are encoded by multiple germ line DNA segments, which are joined into functional variable regions (V_{H}DJ_{H} or V_{K}J_{K}) during B-cell ontogeny. The molecular and genetic diversity of the antibody response to α5β1 was studied in detail.

It should also be appreciated that where a particular antibody differs from its respective germline sequence at the amino acid level, it may be possible to mutate the antibody sequence back to the germline sequence without significant loss of affinity or potency. When such back mutations to germline do not adversely affect affinity or potency of the antibody, they will reduce the risk of immunogenicity of the antibody in human subject. Such corrective mutations can occur at one, two, three or more positions, or a combination of any of the mutated positions, using standard molecular biological techniques. By way of non-limiting example, Table 13 shows that the light chain sequence of mAb 3C2.2A8 (SEQ ID NO.: 20) differs from the corresponding germline sequence (SEQ ID NO.: 62) through a Tyr to Phe mutation (mutation 1) in the FR2 region, a Gln to H is mutation (mutation 2) in the FR2 region. Thus, the amino acid or nucleotide sequence encoding the light chain of mAb 3C2.2A8 can be modified to change mutation 1 to yield the germline sequence at the site of mutation 1. Further, the amino acid or nucleotide sequence encoding the light chain of mAb 3C2.2A8 can be modified to change mutation 2 to yield the germline sequence at the site of mutation 2. Still further, the amino acid or nucleotide sequence encoding the light chain of mAb 3C2.2A8 can be modified to change both mutation 1 and mutation 2 to yield the germline sequence at those particular sites. Tables 8-11 below illustrate the positions of such variations from the germline for mAb 3C5 and 5B11. Each row represents a unique combination of germline and non-germline residues at the position indicated by bold type

TABLE 8
Exemplary Mutations of mAB 5B11 Light Chain (SEQ ID NO: 50)
to Germline at the Indicated Residue Number

| 30 | 61 | 74 | 107 |
|---|---|---|---|
| S | R | T | K |
| N | R | T | K |
| S | N | T | K |
| N | N | T | K |
| S | R | S | K |
| N | R | S | K |
| S | N | S | K |
| N | N | S | K |
| S | R | T | R |
| N | R | T | R |
| S | N | T | R |
| N | N | T | R |
| S | R | S | R |
| N | R | S | R |
| S | N | S | R |
| N | N | S | R |

TABLE 9
Exemplary Mutations of mAB 5B11 Heavy Chain (SEQ ID NO: 48)
to Germline at the Indicated Residue Number

| 18 | 40 | 50 | 56 | 57 | 58 | 99 | 100 | 101 | 103 |
|---|---|---|---|---|---|---|---|---|---|
| L | A | V | S | N | K | — | — | — | S |
| V | A | V | S | N | K | — | — | — | S |
| L | T | V | S | N | K | — | — | — | S |
| V | T | V | S | N | K | — | — | — | S |
| L | A | I | S | N | K | — | — | — | S |
| V | A | I | S | N | K | — | — | — | S |
| L | T | I | S | N | K | — | — | — | S |
| V | T | I | S | N | K | — | — | — | S |
| L | A | V | G | N | K | — | — | — | S |
| V | A | V | G | N | K | — | — | — | S |
| L | T | V | G | N | K | — | — | — | S |
| V | T | V | G | N | K | — | — | — | S |
| L | A | I | G | N | K | — | — | — | S |
| V | A | I | G | N | K | — | — | — | S |
| L | T | I | G | N | K | — | — | — | S |
| V | T | I | G | N | K | — | — | — | S |
| L | A | V | S | I | K | — | — | — | S |
| V | A | V | S | I | K | — | — | — | S |
| L | T | V | S | I | K | — | — | — | S |
| V | T | V | S | I | K | — | — | — | S |
| L | A | I | S | I | K | — | — | — | S |
| V | A | I | S | I | K | — | — | — | S |
| L | T | I | S | I | K | — | — | — | S |
| V | T | I | S | I | K | — | — | — | S |
| L | A | V | G | I | K | — | — | — | S |
| V | A | V | G | I | K | — | — | — | S |
| L | T | V | G | I | K | — | — | — | S |
| V | T | V | G | I | K | — | — | — | S |
| L | A | I | G | I | K | — | — | — | S |
| V | A | I | G | I | K | — | — | — | S |
| L | T | I | G | I | K | — | — | — | S |
| V | T | I | G | I | K | — | — | — | S |
| L | A | V | S | N | E | — | — | — | S |
| V | A | V | S | N | E | — | — | — | S |
| L | T | V | S | N | E | — | — | — | S |
| V | T | V | S | N | E | — | — | — | S |
| L | A | I | S | N | E | — | — | — | S |
| V | A | I | S | N | E | — | — | — | S |
| L | T | I | S | N | E | — | — | — | S |
| V | T | I | S | N | E | — | — | — | S |
| L | A | V | G | N | E | — | — | — | S |
| V | A | V | G | N | E | — | — | — | S |
| L | T | V | G | N | E | — | — | — | S |
| V | T | V | G | N | E | — | — | — | S |
| L | A | I | G | N | E | — | — | — | S |
| V | A | I | G | N | E | — | — | — | S |
| L | T | I | G | N | E | — | — | — | S |
| V | T | I | G | N | E | — | — | — | S |
| L | A | V | S | I | E | — | — | — | S |
| V | A | V | S | I | E | — | — | — | S |
| L | T | V | S | I | E | — | — | — | S |
| V | T | V | S | I | E | — | — | — | S |
| L | A | I | S | I | E | — | — | — | S |
| V | A | I | S | I | E | — | — | — | S |
| L | T | I | S | I | E | — | — | — | S |
| V | T | I | S | I | E | — | — | — | S |
| L | A | V | G | I | E | — | — | — | S |
| V | A | V | G | I | E | — | — | — | S |
| L | T | V | G | I | E | — | — | — | S |
| V | T | V | G | I | E | — | — | — | S |
| L | A | I | G | I | E | — | — | — | S |
| V | A | I | G | I | E | — | — | — | S |
| L | T | I | G | I | E | — | — | — | S |
| V | T | I | G | I | E | — | — | — | S |
| L | A | V | S | N | K | D | — | — | S |
| V | A | V | S | N | K | D | — | — | S |
| L | T | V | S | N | K | D | — | — | S |
| V | T | V | S | N | K | D | — | — | S |
| L | A | I | S | N | K | D | — | — | S |
| V | A | I | S | N | K | D | — | — | S |
| L | T | I | S | N | K | D | — | — | S |
| V | T | I | S | N | K | D | — | — | S |
| L | A | V | G | N | K | D | — | — | S |
| V | A | V | G | N | K | D | — | — | S |
| L | T | V | G | N | K | D | — | — | S |
| V | T | V | G | N | K | D | — | — | S |
| L | A | I | G | N | K | D | — | — | S |
| V | A | I | G | N | K | D | — | — | S |
| L | T | I | G | N | K | D | — | — | S |
| V | T | I | G | N | K | D | — | — | S |
| L | A | V | S | I | K | D | — | — | S |
| V | A | V | S | I | K | D | — | — | S |
| L | T | V | S | I | K | D | — | — | S |
| V | T | V | S | I | K | D | — | — | S |
| L | A | I | S | I | K | D | — | — | S |
| V | A | I | S | I | K | D | — | — | S |
| L | T | I | S | I | K | D | — | — | S |
| V | T | I | S | I | K | D | — | — | S |
| L | A | V | G | I | K | D | — | — | S |
| V | A | V | G | I | K | D | — | — | S |
| L | T | V | G | I | K | D | — | — | S |
| V | T | V | G | I | K | D | — | — | S |
| L | A | I | G | I | K | D | — | — | S |
| V | A | I | G | I | K | D | — | — | S |
| L | T | I | G | I | K | D | — | — | S |
| V | T | I | G | I | K | D | — | — | S |
| L | A | V | S | N | E | D | — | — | S |
| V | A | V | S | N | E | D | — | — | S |
| L | T | V | S | N | E | D | — | — | S |
| V | T | V | S | N | E | D | — | — | S |
| L | A | I | S | N | E | D | — | — | S |
| V | A | I | S | N | E | D | — | — | S |
| L | T | I | S | N | E | D | — | — | S |
| V | T | I | S | N | E | D | — | — | S |
| L | A | V | G | N | E | D | — | — | S |
| V | A | V | G | N | E | D | — | — | S |
| L | T | V | G | N | E | D | — | — | S |
| V | T | V | G | N | E | D | — | — | S |
| L | A | I | G | N | E | D | — | — | S |
| V | A | I | G | N | E | D | — | — | S |
| L | T | I | G | N | E | D | — | — | S |
| V | T | I | G | N | E | D | — | — | S |
| L | A | V | S | I | E | D | — | — | S |
| V | A | V | S | I | E | D | — | — | S |
| L | T | V | S | I | E | D | — | — | S |
| V | T | V | S | I | E | D | — | — | S |
| L | A | I | S | I | E | D | — | — | S |
| V | A | I | S | I | E | D | — | — | S |
| L | T | I | S | I | E | D | — | — | S |
| V | T | I | S | I | E | D | — | — | S |
| L | A | V | G | I | E | D | — | — | S |
| V | A | V | G | I | E | D | — | — | S |
| L | T | V | G | I | E | D | — | — | S |
| V | T | V | G | I | E | D | — | — | S |
| L | A | I | G | I | E | D | — | — | S |
| V | A | I | G | I | E | D | — | — | S |
| L | T | I | G | I | E | D | — | — | S |
| V | T | I | G | I | E | D | — | — | S |
| L | A | V | S | N | K | — | H | — | S |
| V | A | V | S | N | K | — | H | — | S |
| L | T | V | S | N | K | — | H | — | S |
| V | T | V | S | N | K | — | H | — | S |
| L | A | I | S | N | K | — | H | — | S |
| V | A | I | S | N | K | — | H | — | S |
| L | T | I | S | N | K | — | H | — | S |
| V | T | I | S | N | K | — | H | — | S |
| L | A | V | G | N | K | — | H | — | S |
| V | A | V | G | N | K | — | H | — | S |
| L | T | V | G | N | K | — | H | — | S |
| V | T | V | G | N | K | — | H | — | S |
| L | A | I | G | N | K | — | H | — | S |
| V | A | I | G | N | K | — | H | — | S |
| L | T | I | G | N | K | — | H | — | S |
| V | T | I | G | N | K | — | H | — | S |
| L | A | V | S | I | K | — | H | — | S |
| V | A | V | S | I | K | — | H | — | S |
| L | T | V | S | I | K | — | H | — | S |
| V | T | V | S | I | K | — | H | — | S |
| L | A | I | S | I | K | — | H | — | S |
| V | A | I | S | I | K | — | H | — | S |
| L | T | I | S | I | K | — | H | — | S |
| V | T | I | S | I | K | — | H | — | S |
| L | A | V | G | I | K | — | H | — | S |
| V | A | V | G | I | K | — | H | — | S |
| L | T | V | G | I | K | — | H | — | S |
| V | T | V | G | I | K | — | H | — | S |
| L | A | I | G | I | K | — | H | — | S |
| V | A | I | G | I | K | — | H | — | S |
| L | T | I | G | I | K | — | H | — | S |
| V | T | I | G | I | K | — | H | — | S |
| L | A | V | S | N | E | — | H | — | S |
| V | A | V | S | N | E | — | H | — | S |
| L | T | V | S | N | E | — | H | — | S |
| V | T | V | S | N | E | — | H | — | S |
| L | A | I | S | N | E | — | H | — | S |
| V | A | I | S | N | E | — | H | — | S |
| L | T | I | S | N | E | — | H | — | S |
| V | T | I | S | N | E | — | H | — | S |
| L | A | V | G | N | E | — | H | — | S |
| V | A | V | G | N | E | — | H | — | S |
| L | T | V | G | N | E | — | H | — | S |
| V | T | V | G | N | E | — | H | — | S |
| L | A | I | G | N | E | — | H | — | S |
| V | A | I | G | N | E | — | H | — | S |
| L | T | I | G | N | E | — | H | — | S |
| V | T | I | G | N | E | — | H | — | S |
| L | A | V | S | I | E | — | H | — | S |
| V | A | V | S | I | E | — | H | — | S |
| L | T | V | S | I | E | — | H | — | S |
| V | T | V | S | I | E | — | H | — | S |
| L | A | I | S | I | E | — | H | — | S |
| V | A | I | S | I | E | — | H | — | S |
| L | T | I | S | I | E | — | H | — | S |
| V | T | I | S | I | E | — | H | — | S |
| L | A | V | G | I | E | — | H | — | S |
| V | A | V | G | I | E | — | H | — | S |
| L | T | V | G | I | E | — | H | — | S |
| V | T | V | G | I | E | — | H | — | S |
| L | A | I | G | I | E | — | H | — | S |
| V | A | I | G | I | E | — | H | — | S |
| L | T | I | G | I | E | — | H | — | S |
| V | T | I | G | I | E | — | H | — | S |
| L | A | V | S | N | K | D | H | — | S |
| V | A | V | S | N | K | D | H | — | S |
| L | T | V | S | N | K | D | H | — | S |
| V | T | V | S | N | K | D | H | — | S |
| L | A | I | S | N | K | D | H | — | S |
| V | A | I | S | N | K | D | H | — | S |
| L | T | I | S | N | K | D | H | — | S |
| V | T | I | S | N | K | D | H | — | S |
| L | A | V | G | N | K | D | H | — | S |
| V | A | V | G | N | K | D | H | — | S |
| L | T | V | G | N | K | D | H | — | S |
| V | T | V | G | N | K | D | H | — | S |
| L | A | I | G | N | K | D | H | — | S |
| V | A | I | G | N | K | D | H | — | S |
| L | T | I | G | N | K | D | H | — | S |
| V | T | I | G | N | K | D | H | — | S |
| L | A | V | S | I | K | D | H | — | S |
| V | A | V | S | I | K | D | H | — | S |
| L | T | V | S | I | K | D | H | — | S |
| V | T | V | S | I | K | D | H | — | S |
| L | A | I | S | I | K | D | H | — | S |
| V | A | I | S | I | K | D | H | — | S |
| L | T | I | S | I | K | D | H | — | S |
| V | T | I | S | I | K | D | H | — | S |
| L | A | V | G | I | K | D | H | — | S |
| V | A | V | G | I | K | D | H | — | S |
| L | T | V | G | I | K | D | H | — | S |
| V | T | V | G | I | K | D | H | — | S |
| L | A | I | G | I | K | D | H | — | S |
| V | A | I | G | I | K | D | H | — | S |
| L | T | I | G | I | K | D | H | — | S |
| V | T | I | G | I | K | D | H | — | S |
| L | A | V | S | N | E | D | H | — | S |
| V | A | V | S | N | E | D | H | — | S |
| L | T | V | S | N | E | D | H | — | S |
| V | T | V | S | N | E | D | H | — | S |
| L | A | I | S | N | E | D | H | — | S |
| V | A | I | S | N | E | D | H | — | S |
| L | T | I | S | N | E | D | H | — | S |
| V | T | I | S | N | E | D | H | — | S |
| L | A | V | G | N | E | D | H | — | S |
| V | A | V | G | N | E | D | H | — | S |
| L | T | V | G | N | E | D | H | — | S |
| V | T | V | G | N | E | D | H | — | S |
| L | A | I | G | N | E | D | H | — | S |
| V | A | I | G | N | E | D | H | — | S |
| L | T | I | G | N | E | D | H | — | S |
| V | T | I | G | N | E | D | H | — | S |
| L | A | V | S | I | E | D | H | — | S |
| V | A | V | S | I | E | D | H | — | S |
| L | T | V | S | I | E | D | H | — | S |
| V | T | V | S | I | E | D | H | — | S |
| L | A | I | S | I | E | D | H | — | S |
| V | A | I | S | I | E | D | H | — | S |
| L | T | I | S | I | E | D | H | — | S |
| V | T | I | S | I | E | D | H | — | S |
| L | A | V | G | I | E | D | H | — | S |
| V | A | V | G | I | E | D | H | — | S |
| L | T | V | G | I | E | D | H | — | S |
| V | T | V | G | I | E | D | H | — | S |
| L | A | I | G | I | E | D | H | — | S |
| V | A | I | G | I | E | D | H | — | S |
| L | T | I | G | I | E | D | H | — | S |
| V | T | I | G | I | E | D | H | — | S |
| L | A | V | S | N | K | — | — | R | S |
| V | A | V | S | N | K | — | — | R | S |
| L | T | V | S | N | K | — | — | R | S |
| V | T | V | S | N | K | — | — | R | S |
| L | A | I | S | N | K | — | — | R | S |
| V | A | I | S | N | K | — | — | R | S |
| L | T | I | S | N | K | — | — | R | S |
| V | T | I | S | N | K | — | — | R | S |
| L | A | V | G | N | K | — | — | R | S |
| V | A | V | G | N | K | — | — | R | S |
| L | T | V | G | N | K | — | — | R | S |
| V | T | V | G | N | K | — | — | R | S |
| L | A | I | G | N | K | — | — | R | S |
| V | A | I | G | N | K | — | — | R | S |
| L | T | I | G | N | K | — | — | R | S |
| V | T | I | G | N | K | — | — | R | S |
| L | A | V | S | I | K | — | — | R | S |
| V | A | V | S | I | K | — | — | R | S |
| L | T | V | S | I | K | — | — | R | S |
| V | T | V | S | I | K | — | — | R | S |
| L | A | I | S | I | K | — | — | R | S |
| V | A | I | S | I | K | — | — | R | S |
| L | T | I | S | I | K | — | — | R | S |
| V | T | I | S | I | K | — | — | R | S |
| L | A | V | G | I | K | — | — | R | S |
| V | A | V | G | I | K | — | — | R | S |
| L | T | V | G | I | K | — | — | R | S |
| V | T | V | G | I | K | — | — | R | S |
| L | A | I | G | I | K | — | — | R | S |
| V | A | I | G | I | K | — | — | R | S |
| L | T | I | G | I | K | — | — | R | S |
| V | T | I | G | I | K | — | — | R | S |
| L | A | V | S | N | E | — | — | R | S |
| V | A | V | S | N | E | — | — | R | S |
| L | T | V | S | N | E | — | — | R | S |
| V | T | V | S | N | E | — | — | R | S |
| L | A | I | S | N | E | — | — | R | S |
| V | A | I | S | N | E | — | — | R | S |
| L | T | I | S | N | E | — | — | R | S |
| V | T | I | S | N | E | — | — | R | S |
| L | A | V | G | N | E | — | — | R | S |
| V | A | V | G | N | E | — | — | R | S |
| L | T | V | G | N | E | — | — | R | S |
| V | T | V | G | N | E | — | — | R | S |
| L | A | I | G | N | E | — | — | R | S |
| V | A | I | G | N | E | — | — | R | S |
| L | T | I | G | N | E | — | — | R | S |
| V | T | I | G | N | E | — | — | R | S |
| L | A | V | S | I | E | — | — | R | S |
| V | A | V | S | I | E | — | — | R | S |
| L | T | V | S | I | E | — | — | R | S |
| V | T | V | S | I | E | — | — | R | S |
| L | A | I | S | I | E | — | — | R | S |
| V | A | I | S | I | E | — | — | R | S |
| L | T | I | S | I | E | — | — | R | S |
| V | T | I | S | I | E | — | — | R | S |
| L | A | V | G | I | E | — | — | R | S |
| V | A | V | G | I | E | — | — | R | S |
| L | T | V | G | I | E | — | — | R | S |
| V | T | V | G | I | E | — | — | R | S |
| L | A | I | G | I | E | — | — | R | S |
| V | A | I | G | I | E | — | — | R | S |
| L | T | I | G | I | E | — | — | R | S |
| V | T | I | G | I | E | — | — | R | S |
| L | A | V | S | N | K | D | — | R | S |
| V | A | V | S | N | K | D | — | R | S |
| L | T | V | S | N | K | D | — | R | S |
| V | T | V | S | N | K | D | — | R | S |
| L | A | I | S | N | K | D | — | R | S |
| V | A | I | S | N | K | D | — | R | S |
| L | T | I | S | N | K | D | — | R | S |
| V | T | I | S | N | K | D | — | R | S |
| L | A | V | G | N | K | D | — | R | S |
| V | A | V | G | N | K | D | — | R | S |
| L | T | V | G | N | K | D | — | R | S |
| V | T | V | G | N | K | D | — | R | S |
| L | A | I | G | N | K | D | — | R | S |
| V | A | I | G | N | K | D | — | R | S |
| L | T | I | G | N | K | D | — | R | S |
| V | T | I | G | N | K | D | — | R | S |
| L | A | V | S | I | K | D | — | R | S |
| V | A | V | S | I | K | D | — | R | S |
| L | T | V | S | I | K | D | — | R | S |
| V | T | V | S | I | K | D | — | R | S |
| L | A | I | S | I | K | D | — | R | S |
| V | A | I | S | I | K | D | — | R | S |
| L | T | I | S | I | K | D | — | R | S |
| V | T | I | S | I | K | D | — | R | S |
| L | A | V | G | I | K | D | — | R | S |
| V | A | V | G | I | K | D | — | R | S |
| L | T | V | G | I | K | D | — | R | S |
| V | T | V | G | I | K | D | — | R | S |
| L | A | I | G | I | K | D | — | R | S |
| V | A | I | G | I | K | D | — | R | S |
| L | T | I | G | I | K | D | — | R | S |
| V | T | I | G | I | K | D | — | R | S |
| L | A | V | S | N | E | D | — | R | S |
| V | A | V | S | N | E | D | — | R | S |
| L | T | V | S | N | E | D | — | R | S |
| V | T | V | S | N | E | D | — | R | S |
| L | A | I | S | N | E | D | — | R | S |
| V | A | I | S | N | E | D | — | R | S |
| L | T | I | S | N | E | D | — | R | S |
| V | T | I | S | N | E | D | — | R | S |
| L | A | V | G | N | E | D | — | R | S |
| V | A | V | G | N | E | D | — | R | S |
| L | T | V | G | N | E | D | — | R | S |
| V | T | V | G | N | E | D | — | R | S |
| L | A | I | G | N | E | D | — | R | S |
| V | A | I | G | N | E | D | — | R | S |
| L | T | I | G | N | E | D | — | R | S |
| V | T | I | G | N | E | D | — | R | S |
| L | A | V | S | I | E | D | — | R | S |
| V | A | V | S | I | E | D | — | R | S |
| L | T | V | S | I | E | D | — | R | S |
| V | T | V | S | I | E | D | — | R | S |
| L | A | I | S | I | E | D | — | R | S |
| V | A | I | S | I | E | D | — | R | S |
| L | T | I | S | I | E | D | — | R | S |
| V | T | I | S | I | E | D | — | R | S |
| L | A | V | G | I | E | D | — | R | S |
| V | A | V | G | I | E | D | — | R | S |
| L | T | V | G | I | E | D | — | R | S |
| V | T | V | G | I | E | D | — | R | S |
| L | A | I | G | I | E | D | — | R | S |
| V | A | I | G | I | E | D | — | R | S |
| L | T | I | G | I | E | D | — | R | S |
| V | T | I | G | I | E | D | — | R | S |
| L | A | V | S | N | K | — | H | R | S |
| V | A | V | S | N | K | — | H | R | S |
| L | T | V | S | N | K | — | H | R | S |
| V | T | V | S | N | K | — | H | R | S |
| L | A | I | S | N | K | — | H | R | S |
| V | A | I | S | N | K | — | H | R | S |
| L | T | I | S | N | K | — | H | R | S |
| V | T | I | S | N | K | — | H | R | S |
| L | A | V | G | N | K | — | H | R | S |
| V | A | V | G | N | K | — | H | R | S |
| L | T | V | G | N | K | — | H | R | S |
| V | T | V | G | N | K | — | H | R | S |
| L | A | I | G | N | K | — | H | R | S |
| V | A | I | G | N | K | — | H | R | S |
| L | T | I | G | N | K | — | H | R | S |
| V | T | I | G | N | K | — | H | R | S |
| L | A | V | S | I | K | — | H | R | S |
| V | A | V | S | I | K | — | H | R | S |
| L | T | V | S | I | K | — | H | R | S |
| V | T | V | S | I | K | — | H | R | S |
| L | A | I | S | I | K | — | H | R | S |
| V | A | I | S | I | K | — | H | R | S |
| L | T | I | S | I | K | — | H | R | S |
| V | T | I | S | I | K | — | H | R | S |
| L | A | V | G | I | K | — | H | R | S |
| V | A | V | G | I | K | — | H | R | S |
| L | T | V | G | I | K | — | H | R | S |
| V | T | V | G | I | K | — | H | R | S |
| L | A | I | G | I | K | — | H | R | S |
| V | A | I | G | I | K | — | H | R | S |
| L | T | I | G | I | K | — | H | R | S |
| V | T | I | G | I | K | — | H | R | S |
| L | A | V | S | N | E | — | H | R | S |
| V | A | V | S | N | E | — | H | R | S |
| L | T | V | S | N | E | — | H | R | S |
| V | T | V | S | N | E | — | H | R | S |
| L | A | I | S | N | E | — | H | R | S |
| V | A | I | S | N | E | — | H | R | S |
| L | T | I | S | N | E | — | H | R | S |
| V | T | I | S | N | E | — | H | R | S |
| L | A | V | G | N | E | — | H | R | S |
| V | A | V | G | N | E | — | H | R | S |
| L | T | V | G | N | E | — | H | R | S |
| V | T | V | G | N | E | — | H | R | S |
| L | A | I | G | N | E | — | H | R | S |
| V | A | I | G | N | E | — | H | R | S |
| L | T | I | G | N | E | — | H | R | S |
| V | T | I | G | N | E | — | H | R | S |
| L | A | V | S | I | E | — | H | R | S |
| V | A | V | S | I | E | — | H | R | S |
| L | T | V | S | I | E | — | H | R | S |
| V | T | V | S | I | E | — | H | R | S |
| L | A | I | S | I | E | — | H | R | S |
| V | A | I | S | I | E | — | H | R | S |
| L | T | I | S | I | E | — | H | R | S |
| V | T | I | S | I | E | — | H | R | S |
| L | A | V | G | I | E | — | H | R | S |
| V | A | V | G | I | E | — | H | R | S |
| L | T | V | G | I | E | — | H | R | S |
| V | T | V | G | I | E | — | H | R | S |
| L | A | I | G | I | E | — | H | R | S |
| V | A | I | G | I | E | — | H | R | S |
| L | T | I | G | I | E | — | H | R | S |
| V | T | I | G | I | E | — | H | R | S |
| L | A | V | S | N | K | D | H | R | S |
| V | A | V | S | N | K | D | H | R | S |
| L | T | V | S | N | K | D | H | R | S |
| V | T | V | S | N | K | D | H | R | S |
| L | A | I | S | N | K | D | H | R | S |
| V | A | I | S | N | K | D | H | R | S |
| L | T | I | S | N | K | D | H | R | S |
| V | T | I | S | N | K | D | H | R | S |
| L | A | V | G | N | K | D | H | R | S |
| V | A | V | G | N | K | D | H | R | S |
| L | T | V | G | N | K | D | H | R | S |
| V | T | V | G | N | K | D | H | R | S |
| L | A | I | G | N | K | D | H | R | S |
| V | A | I | G | N | K | D | H | R | S |
| L | T | I | G | N | K | D | H | R | S |
| V | T | I | G | N | K | D | H | R | S |
| L | A | V | S | I | K | D | H | R | S |
| V | A | V | S | I | K | D | H | R | S |
| L | T | V | S | I | K | D | H | R | S |
| V | T | V | S | I | K | D | H | R | S |
| L | A | I | S | I | K | D | H | R | S |
| V | A | I | S | I | K | D | H | R | S |
| L | T | I | S | I | K | D | H | R | S |
| V | T | I | S | I | K | D | H | R | S |
| L | A | V | G | I | K | D | H | R | S |
| V | A | V | G | I | K | D | H | R | S |
| L | T | V | G | I | K | D | H | R | S |
| V | T | V | G | I | K | D | H | R | S |
| L | A | I | G | I | K | D | H | R | S |
| V | A | I | G | I | K | D | H | R | S |
| L | T | I | G | I | K | D | H | R | S |
| V | T | I | G | I | K | D | H | R | S |
| L | A | V | S | N | E | D | H | R | S |
| V | A | V | S | N | E | D | H | R | S |
| L | T | V | S | N | E | D | H | R | S |
| V | T | V | S | N | E | D | H | R | S |
| L | A | I | S | N | E | D | H | R | S |
| V | A | I | S | N | E | D | H | R | S |
| L | T | I | S | N | E | D | H | R | S |
| V | T | I | S | N | E | D | H | R | S |
| L | A | V | G | N | E | D | H | R | S |
| V | A | V | G | N | E | D | H | R | S |
| L | T | V | G | N | E | D | H | R | S |
| V | T | V | G | N | E | D | H | R | S |
| L | A | I | G | N | E | D | H | R | S |
| V | A | I | G | N | E | D | H | R | S |
| L | T | I | G | N | E | D | H | R | S |
| V | T | I | G | N | E | D | H | R | S |
| L | A | V | S | I | E | D | H | R | S |
| V | A | V | S | I | E | D | H | R | S |
| L | T | V | S | I | E | D | H | R | S |
| V | T | V | S | I | E | D | H | R | S |
| L | A | I | S | I | E | D | H | R | S |
| V | A | I | S | I | E | D | H | R | S |
| L | T | I | S | I | E | D | H | R | S |
| V | T | I | S | I | E | D | H | R | S |
| L | A | V | G | I | E | D | H | R | S |
| V | A | V | G | I | E | D | H | R | S |
| L | T | V | G | I | E | D | H | R | S |
| V | T | V | G | I | E | D | H | R | S |
| L | A | I | G | I | E | D | H | R | S |
| V | A | I | G | I | E | D | H | R | S |
| L | T | I | G | I | E | D | H | R | S |
| V | T | I | G | I | E | D | H | R | S |
| L | A | V | S | N | K | — | — | — | G |
| V | A | V | S | N | K | — | — | — | G |
| L | T | V | S | N | K | — | — | — | G |
| V | T | V | S | N | K | — | — | — | G |
| L | A | I | S | N | K | — | — | — | G |
| V | A | I | S | N | K | — | — | — | G |
| L | T | I | S | N | K | — | — | — | G |
| V | T | I | S | N | K | — | — | — | G |
| L | A | V | G | N | K | — | — | — | G |
| V | A | V | G | N | K | — | — | — | G |
| L | T | V | G | N | K | — | — | — | G |
| V | T | V | G | N | K | — | — | — | G |
| L | A | I | G | N | K | — | — | — | G |
| V | A | I | G | N | K | — | — | — | G |
| L | T | I | G | N | K | — | — | — | G |
| V | T | I | G | N | K | — | — | — | G |
| L | A | V | S | I | K | — | — | — | G |
| V | A | V | S | I | K | — | — | — | G |
| L | T | V | S | I | K | — | — | — | G |
| V | T | V | S | I | K | — | — | — | G |
| L | A | I | S | I | K | — | — | — | G |
| V | A | I | S | I | K | — | — | — | G |
| L | T | I | S | I | K | — | — | — | G |
| V | T | I | S | I | K | — | — | — | G |
| L | A | V | G | I | K | — | — | — | G |
| V | A | V | G | I | K | — | — | — | G |
| L | T | V | G | I | K | — | — | — | G |
| V | T | V | G | I | K | — | — | — | G |
| L | A | I | G | I | K | — | — | — | G |
| V | A | I | G | I | K | — | — | — | G |
| L | T | I | G | I | K | — | — | — | G |
| V | T | I | G | I | K | — | — | — | G |
| L | A | V | S | N | E | — | — | — | G |
| V | A | V | S | N | E | — | — | — | G |
| L | T | V | S | N | E | — | — | — | G |
| V | T | V | S | N | E | — | — | — | G |
| L | A | I | S | N | E | — | — | — | G |
| V | A | I | S | N | E | — | — | — | G |
| L | T | I | S | N | E | — | — | — | G |
| V | T | I | S | N | E | — | — | — | G |
| L | A | V | G | N | E | — | — | — | G |
| V | A | V | G | N | E | — | — | — | G |
| L | T | V | G | N | E | — | — | — | G |
| V | T | V | G | N | E | — | — | — | G |
| L | A | I | G | N | E | — | — | — | G |
| V | A | I | G | N | E | — | — | — | G |
| L | T | I | G | N | E | — | — | — | G |
| V | T | I | G | N | E | — | — | — | G |
| L | A | V | S | I | E | — | — | — | G |
| V | A | V | S | I | E | — | — | — | G |
| L | T | V | S | I | E | — | — | — | G |
| V | T | V | S | I | E | — | — | — | G |
| L | A | I | S | I | E | — | — | — | G |
| V | A | I | S | I | E | — | — | — | G |
| L | T | I | S | I | E | — | — | — | G |
| V | T | I | S | I | E | — | — | — | G |
| L | A | V | G | I | E | — | — | — | G |
| V | A | V | G | I | E | — | — | — | G |
| L | T | V | G | I | E | — | — | — | G |
| V | T | V | G | I | E | — | — | — | G |
| L | A | I | G | I | E | — | — | — | G |
| V | A | I | G | I | E | — | — | — | G |
| L | T | I | G | I | E | — | — | — | G |
| V | T | I | G | I | E | — | — | — | G |
| L | A | V | S | N | K | D | — | — | G |
| V | A | V | S | N | K | D | — | — | G |
| L | T | V | S | N | K | D | — | — | G |
| V | T | V | S | N | K | D | — | — | G |
| L | A | I | S | N | K | D | — | — | G |
| V | A | I | S | N | K | D | — | — | G |
| L | T | I | S | N | K | D | — | — | G |
| V | T | I | S | N | K | D | — | — | G |
| L | A | V | G | N | K | D | — | — | G |
| V | A | V | G | N | K | D | — | — | G |
| L | T | V | G | N | K | D | — | — | G |
| V | T | V | G | N | K | D | — | — | G |
| L | A | I | G | N | K | D | — | — | G |
| V | A | I | G | N | K | D | — | — | G |
| L | T | I | G | N | K | D | — | — | G |
| V | T | I | G | N | K | D | — | — | G |
| L | A | V | S | I | K | D | — | — | G |
| V | A | V | S | I | K | D | — | — | G |
| L | T | V | S | I | K | D | — | — | G |
| V | T | V | S | I | K | D | — | — | G |
| L | A | I | S | I | K | D | — | — | G |
| V | A | I | S | I | K | D | — | — | G |
| L | T | I | S | I | K | D | — | — | G |
| V | T | I | S | I | K | D | — | — | G |
| L | A | V | G | I | K | D | — | — | G |
| V | A | V | G | I | K | D | — | — | G |
| L | T | V | G | I | K | D | — | — | G |
| V | T | V | G | I | K | D | — | — | G |
| L | A | I | G | I | K | D | — | — | G |
| V | A | I | G | I | K | D | — | — | G |
| L | T | I | G | I | K | D | — | — | G |
| V | T | I | G | I | K | D | — | — | G |
| L | A | V | S | N | E | D | — | — | G |
| V | A | V | S | N | E | D | — | — | G |
| L | T | V | S | N | E | D | — | — | G |
| V | T | V | S | N | E | D | — | — | G |
| L | A | I | S | N | E | D | — | — | G |
| V | A | I | S | N | E | D | — | — | G |
| L | T | I | S | N | E | D | — | — | G |
| V | T | I | S | N | E | D | — | — | G |
| L | A | V | G | N | E | D | — | — | G |
| V | A | V | G | N | E | D | — | — | G |
| L | T | V | G | N | E | D | — | — | G |
| V | T | V | G | N | E | D | — | — | G |
| L | A | I | G | N | E | D | — | — | G |
| V | A | I | G | N | E | D | — | — | G |
| L | T | I | G | N | E | D | — | — | G |
| V | T | I | G | N | E | D | — | — | G |
| L | A | V | S | I | E | D | — | — | G |
| V | A | V | S | I | E | D | — | — | G |
| L | T | V | S | I | E | D | — | — | G |
| V | T | V | S | I | E | D | — | — | G |
| L | A | I | S | I | E | D | — | — | G |
| V | A | I | S | I | E | D | — | — | G |
| L | T | I | S | I | E | D | — | — | G |
| V | T | I | S | I | E | D | — | — | G |
| L | A | V | G | I | E | D | — | — | G |
| V | A | V | G | I | E | D | — | — | G |
| L | T | V | G | I | E | D | — | — | G |
| V | T | V | G | I | E | D | — | — | G |
| L | A | I | G | I | E | D | — | — | G |
| V | A | I | G | I | E | D | — | — | G |
| L | T | I | G | I | E | D | — | — | G |
| V | T | I | G | I | E | D | — | — | G |
| L | A | V | S | N | K | — | H | — | G |
| V | A | V | S | N | K | — | H | — | G |
| L | T | V | S | N | K | — | H | — | G |
| V | T | V | S | N | K | — | H | — | G |
| L | A | I | S | N | K | — | H | — | G |
| V | A | I | S | N | K | — | H | — | G |
| L | T | I | S | N | K | — | H | — | G |
| V | T | I | S | N | K | — | H | — | G |
| L | A | V | G | N | K | — | H | — | G |
| V | A | V | G | N | K | — | H | — | G |
| L | T | V | G | N | K | — | H | — | G |
| V | T | V | G | N | K | — | H | — | G |
| L | A | I | G | N | K | — | H | — | G |
| V | A | I | G | N | K | — | H | — | G |
| L | T | I | G | N | K | — | H | — | G |
| V | T | I | G | N | K | — | H | — | G |
| L | A | V | S | I | K | — | H | — | G |
| V | A | V | S | I | K | — | H | — | G |
| L | T | V | S | I | K | — | H | — | G |
| V | T | V | S | I | K | — | H | — | G |
| L | A | I | S | I | K | — | H | — | G |
| V | A | I | S | I | K | — | H | — | G |
| L | T | I | S | I | K | — | H | — | G |
| V | T | I | S | I | K | — | H | — | G |
| L | A | V | G | I | K | — | H | — | G |
| V | A | V | G | I | K | — | H | — | G |
| L | T | V | G | I | K | — | H | — | G |
| V | T | V | G | I | K | — | H | — | G |
| L | A | I | G | I | K | — | H | — | G |
| V | A | I | G | I | K | — | H | — | G |
| L | T | I | G | I | K | — | H | — | G |
| V | T | I | G | I | K | — | H | — | G |
| L | A | V | S | N | E | — | H | — | G |
| V | A | V | S | N | E | — | H | — | G |
| L | T | V | S | N | E | — | H | — | G |
| V | T | V | S | N | E | — | H | — | G |
| L | A | I | S | N | E | — | H | — | G |
| V | A | I | S | N | E | — | H | — | G |
| L | T | I | S | N | E | — | H | — | G |
| V | T | I | S | N | E | — | H | — | G |
| L | A | V | G | N | E | — | H | — | G |
| V | A | V | G | N | E | — | H | — | G |
| L | T | V | G | N | E | — | H | — | G |
| V | T | V | G | N | E | — | H | — | G |
| L | A | I | G | N | E | — | H | — | G |
| V | A | I | G | N | E | — | H | — | G |
| L | T | I | G | N | E | — | H | — | G |
| V | T | I | G | N | E | — | H | — | G |
| L | A | V | S | I | E | — | H | — | G |
| V | A | V | S | I | E | — | H | — | G |
| L | T | V | S | I | E | — | H | — | G |
| V | T | V | S | I | E | — | H | — | G |
| L | A | I | S | I | E | — | H | — | G |
| V | A | I | S | I | E | — | H | — | G |
| L | T | I | S | I | E | — | H | — | G |
| V | T | I | S | I | E | — | H | — | G |
| L | A | V | G | I | E | — | H | — | G |
| V | A | V | G | I | E | — | H | — | G |
| L | T | V | G | I | E | — | H | — | G |
| V | T | V | G | I | E | — | H | — | G |
| L | A | I | G | I | E | — | H | — | G |
| V | A | I | G | I | E | — | H | — | G |
| L | T | I | G | I | E | — | H | — | G |
| V | T | I | G | I | E | — | H | — | G |
| L | A | V | S | N | K | D | H | — | G |
| V | A | V | S | N | K | D | H | — | G |
| L | T | V | S | N | K | D | H | — | G |
| V | T | V | S | N | K | D | H | — | G |
| L | A | I | S | N | K | D | H | — | G |
| V | A | I | S | N | K | D | H | — | G |
| L | T | I | S | N | K | D | H | — | G |
| V | T | I | S | N | K | D | H | — | G |
| L | A | V | G | N | K | D | H | — | G |
| V | A | V | G | N | K | D | H | — | G |
| L | T | V | G | N | K | D | H | — | G |
| V | T | V | G | N | K | D | H | — | G |
| L | A | I | G | N | K | D | H | — | G |
| V | A | I | G | N | K | D | H | — | G |
| L | T | I | G | N | K | D | H | — | G |
| V | T | I | G | N | K | D | H | — | G |
| L | A | V | S | I | K | D | H | — | G |
| V | A | V | S | I | K | D | H | — | G |
| L | T | V | S | I | K | D | H | — | G |
| V | T | V | S | I | K | D | H | — | G |
| L | A | I | S | I | K | D | H | — | G |
| V | A | I | S | I | K | D | H | — | G |
| L | T | I | S | I | K | D | H | — | G |
| V | T | I | S | I | K | D | H | — | G |
| L | A | V | G | I | K | D | H | — | G |
| V | A | V | G | I | K | D | H | — | G |
| L | T | V | G | I | K | D | H | — | G |
| V | T | V | G | I | K | D | H | — | G |
| L | A | I | G | I | K | D | H | — | G |
| V | A | I | G | I | K | D | H | — | G |
| L | T | I | G | I | K | D | H | — | G |
| V | T | I | G | I | K | D | H | — | G |
| L | A | V | S | N | E | D | H | — | G |
| V | A | V | S | N | E | D | H | — | G |
| L | T | V | S | N | E | D | H | — | G |
| V | T | V | S | N | E | D | H | — | G |
| L | A | I | S | N | E | D | H | — | G |
| V | A | I | S | N | E | D | H | — | G |
| L | T | I | S | N | E | D | H | — | G |
| V | T | I | S | N | E | D | H | — | G |
| L | A | V | G | N | E | D | H | — | G |
| V | A | V | G | N | E | D | H | — | G |
| L | T | V | G | N | E | D | H | — | G |
| V | T | V | G | N | E | D | H | — | G |
| L | A | I | G | N | E | D | H | — | G |
| V | A | I | G | N | E | D | H | — | G |
| L | T | I | G | N | E | D | H | — | G |
| V | T | I | G | N | E | D | H | — | G |
| L | A | V | S | I | E | D | H | — | G |
| V | A | V | S | I | E | D | H | — | G |
| L | T | V | S | I | E | D | H | — | G |
| V | T | V | S | I | E | D | H | — | G |
| L | A | I | S | I | E | D | H | — | G |
| V | A | I | S | I | E | D | H | — | G |
| L | T | I | S | I | E | D | H | — | G |
| V | T | I | S | I | E | D | H | — | G |
| L | A | V | G | I | E | D | H | — | G |
| V | A | V | G | I | E | D | H | — | G |
| L | T | V | G | I | E | D | H | — | G |
| V | T | V | G | I | E | D | H | — | G |
| L | A | I | G | I | E | D | H | — | G |
| V | A | I | G | I | E | D | H | — | G |
| L | T | I | G | I | E | D | H | — | G |
| V | T | I | G | I | E | D | H | — | G |
| L | A | V | S | N | K | — | — | R | G |
| V | A | V | S | N | K | — | — | R | G |
| L | T | V | S | N | K | — | — | R | G |
| V | T | V | S | N | K | — | — | R | G |
| L | A | I | S | N | K | — | — | R | G |
| V | A | I | S | N | K | — | — | R | G |
| L | T | I | S | N | K | — | — | R | G |
| V | T | I | S | N | K | — | — | R | G |
| L | A | V | G | N | K | — | — | R | G |
| V | A | V | G | N | K | — | — | R | G |
| L | T | V | G | N | K | — | — | R | G |
| V | T | V | G | N | K | — | — | R | G |
| L | A | I | G | N | K | — | — | R | G |
| V | A | I | G | N | K | — | — | R | G |
| L | T | I | G | N | K | — | — | R | G |
| V | T | I | G | I | K | — | — | R | G |
| L | A | V | S | I | K | — | — | R | G |
| V | A | V | S | I | K | — | — | R | G |
| L | T | V | S | I | K | — | — | R | G |
| V | T | V | S | I | K | — | — | R | G |
| L | A | I | S | I | K | — | — | R | G |
| V | A | I | S | I | K | — | — | R | G |
| L | T | I | S | I | K | — | — | R | G |
| V | T | I | S | I | K | — | — | R | G |
| L | A | V | G | I | K | — | — | R | G |
| V | A | V | G | I | K | — | — | R | G |
| L | T | V | G | I | K | — | — | R | G |
| V | T | V | G | I | K | — | — | R | G |
| L | A | I | G | I | K | — | — | R | G |
| V | A | I | G | I | K | — | — | R | G |
| L | T | I | G | I | K | — | — | R | G |
| V | T | I | G | I | K | — | — | R | G |
| L | A | V | S | N | E | — | — | R | G |
| V | A | V | S | N | E | — | — | R | G |
| L | T | V | S | N | E | — | — | R | G |
| V | T | V | S | N | E | — | — | R | G |
| L | A | I | S | N | E | — | — | R | G |
| V | A | I | S | N | E | — | — | R | G |
| L | T | I | S | N | E | — | — | R | G |
| V | T | I | S | N | E | — | — | R | G |
| L | A | V | G | N | E | — | — | R | G |
| V | A | V | G | N | E | — | — | R | G |
| L | T | V | G | N | E | — | — | R | G |
| V | T | V | G | N | E | — | — | R | G |
| L | A | I | G | N | E | — | — | R | G |
| V | A | I | G | N | E | — | — | R | G |
| L | T | I | G | N | E | — | — | R | G |
| V | T | I | G | N | E | — | — | R | G |
| L | A | V | S | I | E | — | — | R | G |
| V | A | V | S | I | E | — | — | R | G |
| L | T | V | S | I | E | — | — | R | G |
| V | T | V | S | I | E | — | — | R | G |
| L | A | I | S | I | E | — | — | R | G |
| V | A | I | S | I | E | — | — | R | G |
| L | T | I | S | I | E | — | — | R | G |
| V | T | I | S | I | E | — | — | R | G |
| L | A | V | G | I | E | — | — | R | G |
| V | A | V | G | I | E | — | — | R | G |
| L | T | V | G | I | E | — | — | R | G |
| V | T | V | G | I | E | — | — | R | G |
| L | A | I | G | I | E | — | — | R | G |
| V | A | I | G | I | E | — | — | R | G |
| L | T | I | G | I | E | — | — | R | G |
| V | T | I | G | I | E | — | — | R | G |
| L | A | V | S | N | K | D | — | R | G |
| V | A | V | S | N | K | D | — | R | G |
| L | T | V | S | N | K | D | — | R | G |
| V | T | V | S | N | K | D | — | R | G |
| L | A | I | S | N | K | D | — | R | G |
| V | A | I | S | N | K | D | — | R | G |
| L | T | I | S | N | K | D | — | R | G |
| V | T | I | S | N | K | D | — | R | G |
| L | A | V | G | N | K | D | — | R | G |
| V | A | V | G | N | K | D | — | R | G |
| L | T | V | G | N | K | D | — | R | G |
| V | T | V | G | N | K | D | — | R | G |
| L | A | I | G | N | K | D | — | R | G |
| V | A | I | G | N | K | D | — | R | G |
| L | T | I | G | N | K | D | — | R | G |
| V | T | I | G | N | K | D | — | R | G |
| L | A | V | S | I | K | D | — | R | G |
| V | A | V | S | I | K | D | — | R | G |
| L | T | V | S | I | K | D | — | R | G |
| V | T | V | S | I | K | D | — | R | G |
| L | A | I | S | I | K | D | — | R | G |
| V | A | I | S | I | K | D | — | R | G |
| L | T | I | S | I | K | D | — | R | G |
| V | T | I | S | I | K | D | — | R | G |
| L | A | V | G | I | K | D | — | R | G |
| V | A | V | G | I | K | D | — | R | G |
| L | T | V | G | I | K | D | — | R | G |
| V | T | V | G | I | K | D | — | R | G |
| L | A | I | G | I | K | D | — | R | G |
| V | A | I | G | I | K | D | — | R | G |
| L | T | I | G | I | K | D | — | R | G |
| V | T | I | G | I | K | D | — | R | G |
| L | A | V | S | N | E | D | — | R | G |
| V | A | V | S | N | E | D | — | R | G |
| L | T | V | S | N | E | D | — | R | G |
| V | T | V | S | N | E | D | — | R | G |
| L | A | I | S | N | E | D | — | R | G |
| V | A | I | S | N | E | D | — | R | G |
| L | T | I | S | N | E | D | — | R | G |
| V | T | I | S | N | E | D | — | R | G |
| L | A | V | G | N | E | D | — | R | G |
| V | A | V | G | N | E | D | — | R | G |
| L | T | V | G | N | E | D | — | R | G |
| V | T | V | G | N | E | D | — | R | G |
| L | A | I | G | N | E | D | — | R | G |
| V | A | I | G | N | E | D | — | R | G |
| L | T | I | G | N | E | D | — | R | G |
| V | T | I | G | N | E | D | — | R | G |
| L | A | V | S | I | E | D | — | R | G |
| V | A | V | S | I | E | D | — | R | G |
| L | T | V | S | I | E | D | — | R | G |
| V | T | V | S | I | E | D | — | R | G |
| L | A | I | S | I | E | D | — | R | G |
| V | A | I | S | I | E | D | — | R | G |
| L | T | I | S | I | E | D | — | R | G |
| V | T | I | S | I | E | D | — | R | G |
| L | A | V | G | I | E | D | — | R | G |
| V | A | V | G | I | E | D | — | R | G |
| L | T | V | G | I | E | D | — | R | G |
| V | T | V | G | I | E | D | — | R | G |
| L | A | I | G | I | E | D | — | R | G |
| V | A | I | G | I | E | D | — | R | G |
| L | T | I | G | I | E | D | — | R | G |
| V | T | I | G | I | E | D | — | R | G |
| L | A | V | S | N | K | — | H | R | G |
| V | A | V | S | N | K | — | H | R | G |
| L | T | V | S | N | K | — | H | R | G |
| V | T | V | S | N | K | — | H | R | G |
| L | A | I | S | N | K | — | H | R | G |
| V | A | I | S | N | K | — | H | R | G |
| L | T | I | S | N | K | — | H | R | G |
| V | T | I | S | N | K | — | H | R | G |
| L | A | V | G | N | K | — | H | R | G |
| V | A | V | G | N | K | — | H | R | G |
| L | T | V | G | N | K | — | H | R | G |
| V | T | V | G | N | K | — | H | R | G |
| L | A | I | G | N | K | — | H | R | G |
| V | A | I | G | N | K | — | H | R | G |
| L | T | I | G | N | K | — | H | R | G |
| V | T | I | G | N | K | — | H | R | G |
| L | A | V | S | I | K | — | H | R | G |
| V | A | V | S | I | K | — | H | R | G |
| L | T | V | S | I | K | — | H | R | G |
| V | T | V | S | I | K | — | H | R | G |
| L | A | I | S | I | K | — | H | R | G |
| V | A | I | S | I | K | — | H | R | G |
| L | T | I | S | I | K | — | H | R | G |
| V | T | I | S | I | K | — | H | R | G |
| L | A | V | G | I | K | — | H | R | G |
| V | A | V | G | I | K | — | H | R | G |
| L | T | V | G | I | K | — | H | R | G |
| V | T | V | G | I | K | — | H | R | G |
| L | A | I | G | I | K | — | H | R | G |
| V | A | I | G | I | K | — | H | R | G |
| L | T | I | G | I | K | — | H | R | G |
| V | T | I | G | I | K | — | H | R | G |
| L | A | V | S | N | E | — | H | R | G |
| V | A | V | S | N | E | — | H | R | G |
| L | T | V | S | N | E | — | H | R | G |
| V | T | V | S | N | E | — | H | R | G |
| L | A | I | S | N | E | — | H | R | G |
| V | A | I | S | N | E | — | H | R | G |
| L | T | I | S | N | E | — | H | R | G |
| V | T | I | S | N | E | — | H | R | G |
| L | A | V | G | N | E | — | H | R | G |
| V | A | V | G | N | E | — | H | R | G |
| L | T | V | G | N | E | — | H | R | G |
| V | T | V | G | N | E | — | H | R | G |
| L | A | I | G | N | E | — | H | R | G |
| V | A | I | G | N | E | — | H | R | G |
| L | T | I | G | N | E | — | H | R | G |
| V | T | I | G | N | E | — | H | R | G |
| L | A | V | S | I | E | — | H | R | G |
| V | A | V | S | I | E | — | H | R | G |
| L | T | V | S | I | E | — | H | R | G |
| V | T | V | S | I | E | — | H | R | G |
| L | A | I | S | I | E | — | H | R | G |
| V | A | I | S | I | E | — | H | R | G |
| L | T | I | S | I | E | — | H | R | G |
| V | T | I | S | I | E | — | H | R | G |
| L | A | V | G | I | E | — | H | R | G |
| V | A | V | G | I | E | — | H | R | G |
| L | T | V | G | I | E | — | H | R | G |
| V | T | V | G | I | E | — | H | R | G |
| L | A | I | G | I | E | — | H | R | G |
| V | A | I | G | I | E | — | H | R | G |
| L | T | I | G | I | E | — | H | R | G |
| V | T | I | G | I | E | — | H | R | G |
| L | A | V | S | N | K | D | H | R | G |
| V | A | V | S | N | K | D | H | R | G |
| L | T | V | S | N | K | D | H | R | G |
| V | T | V | S | N | K | D | H | R | G |
| L | A | I | S | N | K | D | H | R | G |
| V | A | I | S | N | K | D | H | R | G |
| L | T | I | S | N | K | D | H | R | G |
| V | T | I | S | N | K | D | H | R | G |
| L | A | V | G | N | K | D | H | R | G |
| V | A | V | G | N | K | D | H | R | G |
| L | T | V | G | N | K | D | H | R | G |
| V | T | V | G | N | K | D | H | R | G |
| L | A | I | G | N | K | D | H | R | G |
| V | A | I | G | N | K | D | H | R | G |
| L | T | I | G | N | K | D | H | R | G |
| V | T | I | G | N | K | D | H | R | G |
| L | A | V | S | I | K | D | H | R | G |
| V | A | V | S | I | K | D | H | R | G |
| L | T | V | S | I | K | D | H | R | G |
| V | T | V | S | I | K | D | H | R | G |
| L | A | I | S | I | K | D | H | R | G |
| V | A | I | S | I | K | D | H | R | G |
| L | T | I | S | I | K | D | H | R | G |
| V | T | I | S | I | K | D | H | R | G |
| L | A | V | G | I | K | D | H | R | G |
| V | A | V | G | I | K | D | H | R | G |
| L | T | V | G | I | K | D | H | R | G |
| V | T | V | G | I | K | D | H | R | G |
| L | A | I | G | I | K | D | H | R | G |
| V | A | I | G | I | K | D | H | R | G |
| L | T | I | G | I | K | D | H | R | G |
| V | T | I | G | I | K | D | H | R | G |
| L | A | V | S | N | E | D | H | R | G |
| V | A | V | S | N | E | D | H | R | G |
| L | T | V | S | N | E | D | H | R | G |
| V | T | V | S | N | E | D | H | R | G |
| L | A | I | S | N | E | D | H | R | G |
| V | A | I | S | N | E | D | H | R | G |
| L | T | I | S | N | E | D | H | R | G |
| V | T | I | S | N | E | D | H | R | G |
| L | A | V | G | N | E | D | H | R | G |
| V | A | V | G | N | E | D | H | R | G |
| L | T | V | G | N | E | D | H | R | G |
| V | T | V | G | N | E | D | H | R | G |
| L | A | I | G | N | E | D | H | R | G |
| V | A | I | G | N | E | D | H | R | G |
| L | T | I | G | N | E | D | H | R | G |
| V | T | I | G | N | E | D | H | R | G |
| L | A | V | S | I | E | D | H | R | G |
| V | A | V | S | I | E | D | H | R | G |
| L | T | V | S | I | E | D | H | R | G |
| V | T | V | S | I | E | D | H | R | G |
| L | A | I | S | I | E | D | H | R | G |
| V | A | I | S | I | E | D | H | R | G |
| L | T | I | S | I | E | D | H | R | G |
| V | T | I | S | I | E | D | H | R | G |
| L | A | V | G | I | E | D | H | R | G |
| V | A | V | G | I | E | D | H | R | G |
| L | T | V | G | I | E | D | H | R | G |
| V | T | V | G | I | E | D | H | R | G |
| L | A | I | G | I | E | D | H | R | G |
| V | A | I | G | I | E | D | H | R | G |
| L | T | I | G | I | E | D | H | R | G |
| V | T | I | G | I | E | D | H | R | G |

“—” indicates the absence of a residue at that position with reference to SEQ ID NO: 48

TABLE 10
Exemplary Mutations of mAB 3C5 Light Chain (SEQ ID NO: 24)
to Germline at the Indicated Residue Number

| 33 | 41 | 56 | 33 |
|---|---|---|---|
| N | Y | G | N |
| S | Y | G | S |
| N | F | G | N |
| S | F | G | S |
| N | Y | S | N |
| S | Y | S | S |
| N | F | S | N |
| S | F | S | S |

TABLE 11
Exemplary Mutations of mAB 3C5 Heavy Chain (SEQ ID NO: 22)
to Germline at the Indicated Residue Number

| 1 | 30 | 35 | 37 | 62 | 98 | 100 | 105 | 106 | 109 |
|---|---|---|---|---|---|---|---|---|---|
| Q | S | Y | S | N | A | V | — | — | Y |
| R | S | Y | S | N | A | V | — | — | Y |
| Q | I | Y | S | N | A | V | — | — | Y |
| R | I | Y | S | N | A | V | — | — | Y |
| Q | S | F | S | N | A | V | — | — | Y |
| R | S | F | S | N | A | V | — | — | Y |
| Q | I | F | S | N | A | V | — | — | Y |
| R | I | F | S | N | A | V | — | — | Y |
| Q | S | Y | T | N | A | V | — | — | Y |
| R | S | Y | T | N | A | V | — | — | Y |
| Q | I | Y | T | N | A | V | — | — | Y |
| R | I | Y | T | N | A | V | — | — | Y |
| Q | S | F | T | N | A | V | — | — | Y |
| R | S | F | T | N | A | V | — | — | Y |
| Q | I | F | T | N | A | V | — | — | Y |
| R | I | F | T | N | A | V | — | — | Y |
| Q | S | Y | S | S | A | V | — | — | Y |
| R | S | Y | S | S | A | V | — | — | Y |
| Q | I | Y | S | S | A | V | — | — | Y |
| R | I | Y | S | S | A | V | — | — | Y |
| Q | S | F | S | S | A | V | — | — | Y |
| R | S | F | S | S | A | V | — | — | Y |
| Q | I | F | S | S | A | V | — | — | Y |
| R | I | F | S | S | A | V | — | — | Y |
| Q | S | Y | T | S | A | V | — | — | Y |
| R | S | Y | T | S | A | V | — | — | Y |
| Q | I | Y | T | S | A | V | — | — | Y |
| R | I | Y | T | S | A | V | — | — | Y |
| Q | S | F | T | S | A | V | — | — | Y |
| R | S | F | T | S | A | V | — | — | Y |
| Q | I | F | T | S | A | V | — | — | Y |
| R | I | F | T | S | A | V | — | — | Y |
| Q | S | Y | S | N | T | V | — | — | Y |
| R | S | Y | S | N | T | V | — | — | Y |
| Q | I | Y | S | N | T | V | — | — | Y |
| R | I | Y | S | N | T | V | — | — | Y |
| Q | S | F | S | N | T | V | — | — | Y |
| R | S | F | S | N | T | V | — | — | Y |
| Q | I | F | S | N | T | V | — | — | Y |
| R | I | F | S | N | T | V | — | — | Y |
| Q | S | Y | T | N | T | V | — | — | Y |
| R | S | Y | T | N | T | V | — | — | Y |
| Q | I | Y | T | N | T | V | — | — | Y |
| R | I | Y | T | N | T | V | — | — | Y |
| Q | S | F | T | N | T | V | — | — | Y |
| R | S | F | T | N | T | V | — | — | Y |
| Q | I | F | T | N | T | V | — | — | Y |
| R | I | F | T | N | T | V | — | — | Y |
| Q | S | Y | S | S | T | V | — | — | Y |
| R | S | Y | S | S | T | V | — | — | Y |
| Q | I | Y | S | S | T | V | — | — | Y |
| R | I | Y | S | S | T | V | — | — | Y |
| Q | S | F | S | S | T | V | — | — | Y |
| R | S | F | S | S | T | V | — | — | Y |
| Q | I | F | S | S | T | V | — | — | Y |
| R | I | F | S | S | T | V | — | — | Y |
| Q | S | Y | T | S | T | V | — | — | Y |
| R | S | Y | T | S | T | V | — | — | Y |
| Q | I | Y | T | S | T | V | — | — | Y |
| R | I | Y | T | S | T | V | — | — | Y |
| Q | S | F | T | S | T | V | — | — | Y |
| R | S | F | T | S | T | V | — | — | Y |
| Q | I | F | T | S | T | V | — | — | Y |
| R | I | F | T | S | T | V | — | — | Y |
| Q | S | Y | S | N | A | A | — | — | Y |
| R | S | Y | S | N | A | A | — | — | Y |
| Q | I | Y | S | N | A | A | — | — | Y |
| R | I | Y | S | N | A | A | — | — | Y |
| Q | S | F | S | N | A | A | — | — | Y |
| R | S | F | S | N | A | A | — | — | Y |
| Q | I | F | S | N | A | A | — | — | Y |
| R | I | F | S | N | A | A | — | — | Y |
| Q | S | Y | T | N | A | A | — | — | Y |
| R | S | Y | T | N | A | A | — | — | Y |
| Q | I | Y | T | N | A | A | — | — | Y |
| R | I | Y | T | N | A | A | — | — | Y |
| Q | S | F | T | N | A | A | — | — | Y |
| R | S | F | T | N | A | A | — | — | Y |
| Q | I | F | T | N | A | A | — | — | Y |
| R | I | F | T | N | A | A | — | — | Y |
| Q | S | Y | S | S | A | A | — | — | Y |
| R | S | Y | S | S | A | A | — | — | Y |
| Q | I | Y | S | S | A | A | — | — | Y |
| R | I | Y | S | S | A | A | — | — | Y |
| Q | S | F | S | S | A | A | — | — | Y |
| R | S | F | S | S | A | A | — | — | Y |
| Q | I | F | S | S | A | A | — | — | Y |
| R | I | F | S | S | A | A | — | — | Y |
| Q | S | Y | T | S | A | A | — | — | Y |
| R | S | Y | T | S | A | A | — | — | Y |
| Q | I | Y | T | S | A | A | — | — | Y |
| R | I | Y | T | S | A | A | — | — | Y |
| Q | S | F | T | S | A | A | — | — | Y |
| R | S | F | T | S | A | A | — | — | Y |
| Q | I | F | T | S | A | A | — | — | Y |
| R | I | F | T | S | A | A | — | — | Y |
| Q | S | Y | S | N | T | A | — | — | Y |
| R | S | Y | S | N | T | A | — | — | Y |
| Q | I | Y | S | N | T | A | — | — | Y |
| R | I | Y | S | N | T | A | — | — | Y |
| Q | S | F | S | N | T | A | — | — | Y |
| R | S | F | S | N | T | A | — | — | Y |
| Q | I | F | S | N | T | A | — | — | Y |
| R | I | F | S | N | T | A | — | — | Y |
| Q | S | Y | T | N | T | A | — | — | Y |
| R | S | Y | T | N | T | A | — | — | Y |
| Q | I | Y | T | N | T | A | — | — | Y |
| R | I | Y | T | N | T | A | — | — | Y |
| Q | S | F | T | N | T | A | — | — | Y |
| R | S | F | T | N | T | A | — | — | Y |
| Q | I | F | T | N | T | A | — | — | Y |
| R | I | F | T | N | T | A | — | — | Y |
| Q | S | Y | S | S | T | A | — | — | Y |
| R | S | Y | S | S | T | A | — | — | Y |
| Q | I | Y | S | S | T | A | — | — | Y |
| R | I | Y | S | S | T | A | — | — | Y |
| Q | S | F | S | S | T | A | — | — | Y |
| R | S | F | S | S | T | A | — | — | Y |
| Q | I | F | S | S | T | A | — | — | Y |
| R | I | F | S | S | T | A | — | — | Y |
| Q | S | Y | T | S | T | A | — | — | Y |
| R | S | Y | T | S | T | A | — | — | Y |
| Q | I | Y | T | S | T | A | — | — | Y |
| R | I | Y | T | S | T | A | — | — | Y |
| Q | S | F | T | S | T | A | — | — | Y |
| R | S | F | T | S | T | A | — | — | Y |
| Q | I | F | T | S | T | A | — | — | Y |
| R | I | F | T | S | T | A | — | — | Y |
| Q | S | Y | S | N | A | V | K | — | Y |
| R | S | Y | S | N | A | V | K | — | Y |
| Q | I | Y | S | N | A | V | K | — | Y |
| R | I | Y | S | N | A | V | K | — | Y |
| Q | S | F | S | N | A | V | K | — | Y |
| R | S | F | S | N | A | V | K | — | Y |
| Q | I | F | S | N | A | V | K | — | Y |
| R | I | F | S | N | A | V | K | — | Y |
| Q | S | Y | T | N | A | V | K | — | Y |
| R | S | Y | T | N | A | V | K | — | Y |
| Q | I | Y | T | N | A | V | K | — | Y |
| R | I | Y | T | N | A | V | K | — | Y |
| Q | S | F | T | N | A | V | K | — | Y |
| R | S | F | T | N | A | V | K | — | Y |
| Q | I | F | T | N | A | V | K | — | Y |
| R | I | F | T | N | A | V | K | — | Y |
| Q | S | Y | S | S | A | V | K | — | Y |
| R | S | Y | S | S | A | V | K | — | Y |
| Q | I | Y | S | S | A | V | K | — | Y |
| R | I | Y | S | S | A | V | K | — | Y |
| Q | S | F | S | S | A | V | K | — | Y |
| R | S | F | S | S | A | V | K | — | Y |
| Q | I | F | S | S | A | V | K | — | Y |
| R | I | F | S | S | A | V | K | — | Y |
| Q | S | Y | T | S | A | V | K | — | Y |
| R | S | Y | T | S | A | V | K | — | Y |
| Q | I | Y | T | S | A | V | K | — | Y |
| R | I | Y | T | S | A | V | K | — | Y |
| Q | S | F | T | S | A | V | K | — | Y |
| R | S | F | T | S | A | V | K | — | Y |
| Q | I | F | T | S | A | V | K | — | Y |
| R | I | F | T | S | A | V | K | — | Y |
| Q | S | Y | S | N | T | V | K | — | Y |
| R | S | Y | S | N | T | V | K | — | Y |
| Q | I | Y | S | N | T | V | K | — | Y |
| R | I | Y | S | N | T | V | K | — | Y |
| Q | S | F | S | N | T | V | K | — | Y |
| R | S | F | S | N | T | V | K | — | Y |
| Q | I | F | S | N | T | V | K | — | Y |
| R | I | F | S | N | T | V | K | — | Y |
| Q | S | Y | T | N | T | V | K | — | Y |
| R | S | Y | T | N | T | V | K | — | Y |
| Q | I | Y | T | N | T | V | K | — | Y |
| R | I | Y | T | N | T | V | K | — | Y |
| Q | S | F | T | N | T | V | K | — | Y |
| R | S | F | T | N | T | V | K | — | Y |
| Q | I | F | T | N | T | V | K | — | Y |
| R | I | F | T | N | T | V | K | — | Y |
| Q | S | Y | S | S | T | V | K | — | Y |
| R | S | Y | S | S | T | V | K | — | Y |
| Q | I | Y | S | S | T | V | K | — | Y |
| R | I | Y | S | S | T | V | K | — | Y |
| Q | S | F | S | S | T | V | K | — | Y |
| R | S | F | S | S | T | V | K | — | Y |
| Q | I | F | S | S | T | V | K | — | Y |
| R | I | F | S | S | T | V | K | — | Y |
| Q | S | Y | T | S | T | V | K | — | Y |
| R | S | Y | T | S | T | V | K | — | Y |
| Q | I | Y | T | S | T | V | K | — | Y |
| R | I | Y | T | S | T | V | K | — | Y |
| Q | S | F | T | S | T | V | K | — | Y |
| R | S | F | T | S | T | V | K | — | Y |
| Q | I | F | T | S | T | V | K | — | Y |
| R | I | F | T | S | T | V | K | — | Y |
| Q | S | Y | S | N | A | A | K | — | Y |
| R | S | Y | S | N | A | A | K | — | Y |
| Q | I | Y | S | N | A | A | K | — | Y |
| R | I | Y | S | N | A | A | K | — | Y |
| Q | S | F | S | N | A | A | K | — | Y |
| R | S | F | S | N | A | A | K | — | Y |
| Q | I | F | S | N | A | A | K | — | Y |
| R | I | F | S | N | A | A | K | — | Y |
| Q | S | Y | T | N | A | A | K | — | Y |
| R | S | Y | T | N | A | A | K | — | Y |
| Q | I | Y | T | N | A | A | K | — | Y |
| R | I | Y | T | N | A | A | K | — | Y |
| Q | S | F | T | N | A | A | K | — | Y |
| R | S | F | T | N | A | A | K | — | Y |
| Q | I | F | T | N | A | A | K | — | Y |
| R | I | F | T | N | A | A | K | — | Y |
| Q | S | Y | S | S | A | A | K | — | Y |
| R | S | Y | S | S | A | A | K | — | Y |
| Q | I | Y | S | S | A | A | K | — | Y |
| R | I | Y | S | S | A | A | K | — | Y |
| Q | S | F | S | S | A | A | K | — | Y |
| R | S | F | S | S | A | A | K | — | Y |
| Q | I | F | S | S | A | A | K | — | Y |
| R | I | F | S | S | A | A | K | — | Y |
| Q | S | Y | T | S | A | A | K | — | Y |
| R | S | Y | T | S | A | A | K | — | Y |
| Q | I | Y | T | S | A | A | K | — | Y |
| R | I | Y | T | S | A | A | K | — | Y |
| Q | S | F | T | S | A | A | K | — | Y |
| R | S | F | T | S | A | A | K | — | Y |
| Q | I | F | T | S | A | A | K | — | Y |
| R | I | F | T | S | A | A | K | — | Y |
| Q | S | Y | S | N | T | A | K | — | Y |
| R | S | Y | S | N | T | A | K | — | Y |
| Q | I | Y | S | N | T | A | K | — | Y |
| R | I | Y | S | N | T | A | K | — | Y |
| Q | S | F | S | N | T | A | K | — | Y |
| R | S | F | S | N | T | A | K | — | Y |
| Q | I | F | S | N | T | A | K | — | Y |
| R | I | F | S | N | T | A | K | — | Y |
| Q | S | Y | T | N | T | A | K | — | Y |
| R | S | Y | T | N | T | A | K | — | Y |
| Q | I | Y | T | N | T | A | K | — | Y |
| R | I | Y | T | N | T | A | K | — | Y |
| Q | S | F | T | N | T | A | K | — | Y |
| R | S | F | T | N | T | A | K | — | Y |
| Q | I | F | T | N | T | A | K | — | Y |
| R | I | F | T | N | T | A | K | — | Y |
| Q | S | Y | S | S | T | A | K | — | Y |
| R | S | Y | S | S | T | A | K | — | Y |
| Q | I | Y | S | S | T | A | K | — | Y |
| R | I | Y | S | S | T | A | K | — | Y |
| Q | S | F | S | S | T | A | K | — | Y |
| R | S | F | S | S | T | A | K | — | Y |
| Q | I | F | S | S | T | A | K | — | Y |
| R | I | F | S | S | T | A | K | — | Y |
| Q | S | Y | T | S | T | A | K | — | Y |
| R | S | Y | T | S | T | A | K | — | Y |
| Q | I | Y | T | S | T | A | K | — | Y |
| R | I | Y | T | S | T | A | K | — | Y |
| Q | S | F | T | S | T | A | K | — | Y |
| R | S | F | T | S | T | A | K | — | Y |
| Q | I | F | T | S | T | A | K | — | Y |
| R | I | F | T | S | T | A | K | — | Y |
| Q | S | Y | S | N | A | V | — | G | Y |
| R | S | Y | S | N | A | V | — | G | Y |
| Q | I | Y | S | N | A | V | — | G | Y |
| R | I | Y | S | N | A | V | — | G | Y |
| Q | S | F | S | N | A | V | — | G | Y |
| R | S | F | S | N | A | V | — | G | Y |
| Q | I | F | S | N | A | V | — | G | Y |
| R | I | F | S | N | A | V | — | G | Y |
| Q | S | Y | T | N | A | V | — | G | Y |
| R | S | Y | T | N | A | V | — | G | Y |
| Q | I | Y | T | N | A | V | — | G | Y |
| R | I | Y | T | N | A | V | — | G | Y |
| Q | S | F | T | N | A | V | — | G | Y |
| R | S | F | T | N | A | V | — | G | Y |
| Q | I | F | T | N | A | V | — | G | Y |
| R | I | F | T | N | A | V | — | G | Y |
| Q | S | Y | S | S | A | V | — | G | Y |
| R | S | Y | S | S | A | V | — | G | Y |
| Q | I | Y | S | S | A | V | — | G | Y |
| R | I | Y | S | S | A | V | — | G | Y |
| Q | S | F | S | S | A | V | — | G | Y |
| R | S | F | S | S | A | V | — | G | Y |
| Q | I | F | S | S | A | V | — | G | Y |
| R | I | F | S | S | A | V | — | G | Y |
| Q | S | Y | T | S | A | V | — | G | Y |
| R | S | Y | T | S | A | V | — | G | Y |
| Q | I | Y | T | S | A | V | — | G | Y |
| R | I | Y | T | S | A | V | — | G | Y |
| Q | S | F | T | S | A | V | — | G | Y |
| R | S | F | T | S | A | V | — | G | Y |
| Q | I | F | T | S | A | V | — | G | Y |
| R | I | F | T | S | A | V | — | G | Y |
| Q | S | Y | S | N | T | V | — | G | Y |
| R | S | Y | S | N | T | V | — | G | Y |
| Q | I | Y | S | N | T | V | — | G | Y |
| R | I | Y | S | N | T | V | — | G | Y |
| Q | S | F | S | N | T | V | — | G | Y |
| R | S | F | S | N | T | V | — | G | Y |
| Q | I | F | S | N | T | V | — | G | Y |
| R | I | F | S | N | T | V | — | G | Y |
| Q | S | Y | T | N | T | V | — | G | Y |
| R | S | Y | T | N | T | V | — | G | Y |
| Q | I | Y | T | N | T | V | — | G | Y |
| R | I | Y | T | N | T | V | — | G | Y |
| Q | S | F | T | N | T | V | — | G | Y |
| R | S | F | T | N | T | V | — | G | Y |
| Q | I | F | T | N | T | V | — | G | Y |
| R | I | F | T | N | T | V | — | G | Y |
| Q | S | Y | S | S | T | V | — | G | Y |
| R | S | Y | S | S | T | V | — | G | Y |
| Q | I | Y | S | S | T | V | — | G | Y |
| R | I | Y | S | S | T | V | — | G | Y |
| Q | S | F | S | S | T | V | — | G | Y |
| R | S | F | S | S | T | V | — | G | Y |
| Q | I | F | S | S | T | V | — | G | Y |
| R | I | F | S | S | T | V | — | G | Y |
| Q | S | Y | T | S | T | V | — | G | Y |
| R | S | Y | T | S | T | V | — | G | Y |
| Q | I | Y | T | S | T | V | — | G | Y |
| R | I | Y | T | S | T | V | — | G | Y |
| Q | S | F | T | S | T | V | — | G | Y |
| R | S | F | T | S | T | V | — | G | Y |
| Q | I | F | T | S | T | V | — | G | Y |
| R | I | F | T | S | T | V | — | G | Y |
| Q | S | Y | S | N | A | A | — | G | Y |
| R | S | Y | S | N | A | A | — | G | Y |
| Q | I | Y | S | N | A | A | — | G | Y |
| R | I | Y | S | N | A | A | — | G | Y |
| Q | S | F | S | N | A | A | — | G | Y |
| R | S | F | S | N | A | A | — | G | Y |
| Q | I | F | S | N | A | A | — | G | Y |
| R | I | F | S | N | A | A | — | G | Y |
| Q | S | Y | T | N | A | A | — | G | Y |
| R | S | Y | T | N | A | A | — | G | Y |
| Q | I | Y | T | N | A | A | — | G | Y |
| R | I | Y | T | N | A | A | — | G | Y |
| Q | S | F | T | N | A | A | — | G | Y |
| R | S | F | T | N | A | A | — | G | Y |
| Q | I | F | T | N | A | A | — | G | Y |
| R | I | F | T | N | A | A | — | G | Y |
| Q | S | Y | S | S | A | A | — | G | Y |
| R | S | Y | S | S | A | A | — | G | Y |
| Q | I | Y | S | S | A | A | — | G | Y |
| R | I | Y | S | S | A | A | — | G | Y |
| Q | S | F | S | S | A | A | — | G | Y |
| R | S | F | S | S | A | A | — | G | Y |
| Q | I | F | S | S | A | A | — | G | Y |
| R | I | F | S | S | A | A | — | G | Y |
| Q | S | Y | T | S | A | A | — | G | Y |
| R | S | Y | T | S | A | A | — | G | Y |
| Q | I | Y | T | S | A | A | — | G | Y |
| R | I | Y | T | S | A | A | — | G | Y |
| Q | S | F | T | S | A | A | — | G | Y |
| R | S | F | T | S | A | A | — | G | Y |
| Q | I | F | T | S | A | A | — | G | Y |
| R | I | F | T | S | A | A | — | G | Y |
| Q | S | Y | S | N | T | A | — | G | Y |
| R | S | Y | S | N | T | A | — | G | Y |
| Q | I | Y | S | N | T | A | — | G | Y |
| R | I | Y | S | N | T | A | — | G | Y |
| Q | S | F | S | N | T | A | — | G | Y |
| R | S | F | S | N | T | A | — | G | Y |
| Q | I | F | S | N | T | A | — | G | Y |
| R | I | F | S | N | T | A | — | G | Y |
| Q | S | Y | T | N | T | A | — | G | Y |
| R | S | Y | T | N | T | A | — | G | Y |
| Q | I | Y | T | N | T | A | — | G | Y |
| R | I | Y | T | N | T | A | — | G | Y |
| Q | S | F | T | N | T | A | — | G | Y |
| R | S | F | T | N | T | A | — | G | Y |
| Q | I | F | T | N | T | A | — | G | Y |
| R | I | F | T | N | T | A | — | G | Y |
| Q | S | Y | S | S | T | A | — | G | Y |
| R | S | Y | S | S | T | A | — | G | Y |
| Q | I | Y | S | S | T | A | — | G | Y |
| R | I | Y | S | S | T | A | — | G | Y |
| Q | S | F | S | S | T | A | — | G | Y |
| R | S | F | S | S | T | A | — | G | Y |
| Q | I | F | S | S | T | A | — | G | Y |
| R | I | F | S | S | T | A | — | G | Y |
| Q | S | Y | T | S | T | A | — | G | Y |
| R | S | Y | T | S | T | A | — | G | Y |
| Q | I | Y | T | S | T | A | — | G | Y |
| R | I | Y | T | S | T | A | — | G | Y |
| Q | S | F | T | S | T | A | — | G | Y |
| R | S | F | T | S | T | A | — | G | Y |
| Q | I | F | T | S | T | A | — | G | Y |
| R | I | F | T | S | T | A | — | G | Y |
| Q | S | Y | S | N | A | V | K | G | Y |
| R | S | Y | S | N | A | V | K | G | Y |
| Q | I | Y | S | N | A | V | K | G | Y |
| R | I | Y | S | N | A | V | K | G | Y |
| Q | S | F | S | N | A | V | K | G | Y |
| R | S | F | S | N | A | V | K | G | Y |
| Q | I | F | S | N | A | V | K | G | Y |
| R | I | F | S | N | A | V | K | G | Y |
| Q | S | Y | T | N | A | V | K | G | Y |
| R | S | Y | T | N | A | V | K | G | Y |
| Q | I | Y | T | N | A | V | K | G | Y |
| R | I | Y | T | N | A | V | K | G | Y |
| Q | S | F | T | N | A | V | K | G | Y |
| R | S | F | T | N | A | V | K | G | Y |
| Q | I | F | T | N | A | V | K | G | Y |
| R | I | F | T | N | A | V | K | G | Y |
| Q | S | Y | S | S | A | V | K | G | Y |
| R | S | Y | S | S | A | V | K | G | Y |
| Q | I | Y | S | S | A | V | K | G | Y |
| R | I | Y | S | S | A | V | K | G | Y |
| Q | S | F | S | S | A | V | K | G | Y |
| R | S | F | S | S | A | V | K | G | Y |
| Q | I | F | S | S | A | V | K | G | Y |
| R | I | F | S | S | A | V | K | G | Y |
| Q | S | Y | T | S | A | V | K | G | Y |
| R | S | Y | T | S | A | V | K | G | Y |
| Q | I | Y | T | S | A | V | K | G | Y |
| R | I | Y | T | S | A | V | K | G | Y |
| Q | S | F | T | S | A | V | K | G | Y |
| R | S | F | T | S | A | V | K | G | Y |
| Q | I | F | T | S | A | V | K | G | Y |
| R | I | F | T | S | A | V | K | G | Y |
| Q | S | Y | S | N | T | V | K | G | Y |
| R | S | Y | S | N | T | V | K | G | Y |
| Q | I | Y | S | N | T | V | K | G | Y |
| R | I | Y | S | N | T | V | K | G | Y |
| Q | S | F | S | N | T | V | K | G | Y |
| R | S | F | S | N | T | V | K | G | Y |
| Q | I | F | S | N | T | V | K | G | Y |
| R | I | F | S | N | T | V | K | G | Y |
| Q | S | Y | T | N | T | V | K | G | Y |
| R | S | Y | T | N | T | V | K | G | Y |
| Q | I | Y | T | N | T | V | K | G | Y |
| R | I | Y | T | N | T | V | K | G | Y |
| Q | S | F | T | N | T | V | K | G | Y |
| R | S | F | T | N | T | V | K | G | Y |
| Q | I | F | T | N | T | V | K | G | Y |
| R | I | F | T | N | T | V | K | G | Y |
| Q | S | Y | S | S | T | V | K | G | Y |
| R | S | Y | S | S | T | V | K | G | Y |
| Q | I | Y | S | S | T | V | K | G | Y |
| R | I | Y | S | S | T | V | K | G | Y |
| Q | S | F | S | S | T | V | K | G | Y |
| R | S | F | S | S | T | V | K | G | Y |
| Q | I | F | S | S | T | V | K | G | Y |
| R | I | F | S | S | T | V | K | G | Y |
| Q | S | Y | T | S | T | V | K | G | Y |
| R | S | Y | T | S | T | V | K | G | Y |
| Q | I | Y | T | S | T | V | K | G | Y |
| R | I | Y | T | S | T | V | K | G | Y |
| Q | S | F | T | S | T | V | K | G | Y |
| R | S | F | T | S | T | V | K | G | Y |
| Q | I | F | T | S | T | V | K | G | Y |
| R | I | F | T | S | T | V | K | G | Y |
| Q | S | Y | S | N | A | A | K | G | Y |
| R | S | Y | S | N | A | A | K | G | Y |
| Q | I | Y | S | N | A | A | K | G | Y |
| R | I | Y | S | N | A | A | K | G | Y |
| Q | S | F | S | N | A | A | K | G | Y |
| R | S | F | S | N | A | A | K | G | Y |
| Q | I | F | S | N | A | A | K | G | Y |
| R | I | F | S | N | A | A | K | G | Y |
| Q | S | Y | T | N | A | A | K | G | Y |
| R | S | Y | T | N | A | A | K | G | Y |
| Q | I | Y | T | N | A | A | K | G | Y |
| R | I | Y | T | N | A | A | K | G | Y |
| Q | S | F | T | N | A | A | K | G | Y |
| R | S | F | T | N | A | A | K | G | Y |
| Q | I | F | T | N | A | A | K | G | Y |
| R | I | F | T | N | A | A | K | G | Y |
| Q | S | Y | S | S | A | A | K | G | Y |
| R | S | Y | S | S | A | A | K | G | Y |
| Q | I | Y | S | S | A | A | K | G | Y |
| R | I | Y | S | S | A | A | K | G | Y |
| Q | S | F | S | S | A | A | K | G | Y |
| R | S | F | S | S | A | A | K | G | Y |
| Q | I | F | S | S | A | A | K | G | Y |
| R | I | F | S | S | A | A | K | G | Y |
| Q | S | Y | T | S | A | A | K | G | Y |
| R | S | Y | T | S | A | A | K | G | Y |
| Q | I | Y | T | S | A | A | K | G | Y |
| R | I | Y | T | S | A | A | K | G | Y |
| Q | S | F | T | S | A | A | K | G | Y |
| R | S | F | T | S | A | A | K | G | Y |
| Q | I | F | T | S | A | A | K | G | Y |
| R | I | F | T | S | A | A | K | G | Y |
| Q | S | Y | S | N | T | A | K | G | Y |
| R | S | Y | S | N | T | A | K | G | Y |
| Q | I | Y | S | N | T | A | K | G | Y |
| R | I | Y | S | N | T | A | K | G | Y |
| Q | S | F | S | N | T | A | K | G | Y |
| R | S | F | S | N | T | A | K | G | Y |
| Q | I | F | S | N | T | A | K | G | Y |
| R | I | F | S | N | T | A | K | G | Y |
| Q | S | Y | T | N | T | A | K | G | Y |
| R | S | Y | T | N | T | A | K | G | Y |
| Q | I | Y | T | N | T | A | K | G | Y |
| R | I | Y | T | N | T | A | K | G | Y |
| Q | S | F | T | N | T | A | K | G | Y |
| R | S | F | T | N | T | A | K | G | Y |
| Q | I | F | T | N | T | A | K | G | Y |
| R | I | F | T | N | T | A | K | G | Y |
| Q | S | Y | S | S | T | A | K | G | Y |
| R | S | Y | S | S | T | A | K | G | Y |
| Q | I | Y | S | S | T | A | K | G | Y |
| R | I | Y | S | S | T | A | K | G | Y |
| Q | S | F | S | S | T | A | K | G | Y |
| R | S | F | S | S | T | A | K | G | Y |
| Q | I | F | S | S | T | A | K | G | Y |
| R | I | F | S | S | T | A | K | G | Y |
| Q | S | Y | T | S | T | A | K | G | Y |
| R | S | Y | T | S | T | A | K | G | Y |
| Q | I | Y | T | S | T | A | K | G | Y |
| R | I | Y | T | S | T | A | K | G | Y |
| Q | S | F | T | S | T | A | K | G | Y |
| R | S | F | T | S | T | A | K | G | Y |
| Q | I | F | T | S | T | A | K | G | Y |
| R | I | F | T | S | T | A | K | G | Y |
| Q | S | Y | S | N | A | V | — | — | S |
| R | S | Y | S | N | A | V | — | — | S |
| Q | I | Y | S | N | A | V | — | — | S |
| R | I | Y | S | N | A | V | — | — | S |
| Q | S | F | S | N | A | V | — | — | S |
| R | S | F | S | N | A | V | — | — | S |
| Q | I | F | S | N | A | V | — | — | S |
| R | I | F | S | N | A | V | — | — | S |
| Q | S | Y | T | N | A | V | — | — | S |
| R | S | Y | T | N | A | V | — | — | S |
| Q | I | Y | T | N | A | V | — | — | S |
| R | I | Y | T | N | A | V | — | — | S |
| Q | S | F | T | N | A | V | — | — | S |
| R | S | F | T | N | A | V | — | — | S |
| Q | I | F | T | N | A | V | — | — | S |
| R | I | F | T | N | A | V | — | — | S |
| Q | S | Y | S | S | A | V | — | — | S |
| R | S | Y | S | S | A | V | — | — | S |
| Q | I | Y | S | S | A | V | — | — | S |
| R | I | Y | S | S | A | V | — | — | S |
| Q | S | F | S | S | A | V | — | — | S |
| R | S | F | S | S | A | V | — | — | S |
| Q | I | F | S | S | A | V | — | — | S |
| R | I | F | S | S | A | V | — | — | S |
| Q | S | Y | T | S | A | V | — | — | S |
| R | S | Y | T | S | A | V | — | — | S |
| Q | I | Y | T | S | A | V | — | — | S |
| R | I | Y | T | S | A | V | — | — | S |
| Q | S | F | T | S | A | V | — | — | S |
| R | S | F | T | S | A | V | — | — | S |
| Q | I | F | T | S | A | V | — | — | S |
| R | I | F | T | S | A | V | — | — | S |
| Q | S | Y | S | N | T | V | — | — | S |
| R | S | Y | S | N | T | V | — | — | S |
| Q | I | Y | S | N | T | V | — | — | S |
| R | I | Y | S | N | T | V | — | — | S |
| Q | S | F | S | N | T | V | — | — | S |
| R | S | F | S | N | T | V | — | — | S |
| Q | I | F | S | N | T | V | — | — | S |
| R | I | F | S | N | T | V | — | — | S |
| Q | S | Y | T | N | T | V | — | — | S |
| R | S | Y | T | N | T | V | — | — | S |
| Q | I | Y | T | N | T | V | — | — | S |
| R | I | Y | T | N | T | V | — | — | S |
| Q | S | F | T | N | T | V | — | — | S |
| R | S | F | T | N | T | V | — | — | S |
| Q | I | F | T | N | T | V | — | — | S |
| R | I | F | T | N | T | V | — | — | S |
| Q | S | Y | S | S | T | V | — | — | S |
| R | S | Y | S | S | T | V | — | — | S |
| Q | I | Y | S | S | T | V | — | — | S |
| R | I | Y | S | S | T | V | — | — | S |
| Q | S | F | S | S | T | V | — | — | S |
| R | S | F | S | S | T | V | — | — | S |
| Q | I | F | S | S | T | V | — | — | S |
| R | I | F | S | S | T | V | — | — | S |
| Q | S | Y | T | S | T | V | — | — | S |
| R | S | Y | T | S | T | V | — | — | S |
| Q | I | Y | T | S | T | V | — | — | S |
| R | I | Y | T | S | T | V | — | — | S |
| Q | S | F | T | S | T | V | — | — | S |
| R | S | F | T | S | T | V | — | — | S |
| Q | I | F | T | S | T | V | — | — | S |
| R | I | F | T | S | T | V | — | — | S |
| Q | S | Y | S | N | A | A | — | — | S |
| R | S | Y | S | N | A | A | — | — | S |
| Q | I | Y | S | N | A | A | — | — | S |
| R | I | Y | S | N | A | A | — | — | S |
| Q | S | F | S | N | A | A | — | — | S |
| R | S | F | S | N | A | A | — | — | S |
| Q | I | F | S | N | A | A | — | — | S |
| R | I | F | S | N | A | A | — | — | S |
| Q | S | Y | T | N | A | A | — | — | S |
| R | S | Y | T | N | A | A | — | — | S |
| Q | I | Y | T | N | A | A | — | — | S |
| R | I | Y | T | N | A | A | — | — | S |
| Q | S | F | T | N | A | A | — | — | S |
| R | S | F | T | N | A | A | — | — | S |
| Q | I | F | T | N | A | A | — | — | S |
| R | I | F | T | N | A | A | — | — | S |
| Q | S | Y | S | S | A | A | — | — | S |
| R | S | Y | S | S | A | A | — | — | S |
| Q | I | Y | S | S | A | A | — | — | S |
| R | I | Y | S | S | A | A | — | — | S |
| Q | S | F | S | S | A | A | — | — | S |
| R | S | F | S | S | A | A | — | — | S |
| Q | I | F | S | S | A | A | — | — | S |
| R | I | F | S | S | A | A | — | — | S |
| Q | S | Y | T | S | A | A | — | — | S |
| R | S | Y | T | S | A | A | — | — | S |
| Q | I | Y | T | S | A | A | — | — | S |
| R | I | Y | T | S | A | A | — | — | S |
| Q | S | F | T | S | A | A | — | — | S |
| R | S | F | T | S | A | A | — | — | S |
| Q | I | F | T | S | A | A | — | — | S |
| R | I | F | T | S | A | A | — | — | S |
| Q | S | Y | S | N | T | A | — | — | S |
| R | S | Y | S | N | T | A | — | — | S |
| Q | I | Y | S | N | T | A | — | — | S |
| R | I | Y | S | N | T | A | — | — | S |
| Q | S | F | S | N | T | A | — | — | S |
| R | S | F | S | N | T | A | — | — | S |
| Q | I | F | S | N | T | A | — | — | S |
| R | I | F | S | N | T | A | — | — | S |
| Q | S | Y | T | N | T | A | — | — | S |
| R | S | Y | T | N | T | A | — | — | S |
| Q | I | Y | T | N | T | A | — | — | S |
| R | I | Y | T | N | T | A | — | — | S |
| Q | S | F | T | N | T | A | — | — | S |
| R | S | F | T | N | T | A | — | — | S |
| Q | I | F | T | N | T | A | — | — | S |
| R | I | F | T | N | T | A | — | — | S |
| Q | S | Y | S | S | T | A | — | — | S |
| R | S | Y | S | S | T | A | — | — | S |
| Q | I | Y | S | S | T | A | — | — | S |
| R | I | Y | S | S | T | A | — | — | S |
| Q | S | F | S | S | T | A | — | — | S |
| R | S | F | S | S | T | A | — | — | S |
| Q | I | F | S | S | T | A | — | — | S |
| R | I | F | S | S | T | A | — | — | S |
| Q | S | Y | T | S | T | A | — | — | S |
| R | S | Y | T | S | T | A | — | — | S |
| Q | I | Y | T | S | T | A | — | — | S |
| R | I | Y | T | S | T | A | — | — | S |
| Q | S | F | T | S | T | A | — | — | S |
| R | S | F | T | S | T | A | — | — | S |
| Q | I | F | T | S | T | A | — | — | S |
| R | I | F | T | S | T | A | — | — | S |
| Q | S | Y | S | N | A | V | K | — | S |
| R | S | Y | S | N | A | V | K | — | S |
| Q | I | Y | S | N | A | V | K | — | S |
| R | I | Y | S | N | A | V | K | — | S |
| Q | S | F | S | N | A | V | K | — | S |
| R | S | F | S | N | A | V | K | — | S |
| Q | I | F | S | N | A | V | K | — | S |
| R | I | F | S | N | A | V | K | — | S |
| Q | S | Y | T | N | A | V | K | — | S |
| R | S | Y | T | N | A | V | K | — | S |
| Q | I | Y | T | N | A | V | K | — | S |
| R | I | Y | T | N | A | V | K | — | S |
| Q | S | F | T | N | A | V | K | — | S |
| R | S | F | T | N | A | V | K | — | S |
| Q | I | F | T | N | A | V | K | — | S |
| R | I | F | T | N | A | V | K | — | S |
| Q | S | Y | S | S | A | V | K | — | S |
| R | S | Y | S | S | A | V | K | — | S |
| Q | I | Y | S | S | A | V | K | — | S |
| R | I | Y | S | S | A | V | K | — | S |
| Q | S | F | S | S | A | V | K | — | S |
| R | S | F | S | S | A | V | K | — | S |
| Q | I | F | S | S | A | V | K | — | S |
| R | I | F | S | S | A | V | K | — | S |
| Q | S | Y | T | S | A | V | K | — | S |
| R | S | Y | T | S | A | V | K | — | S |
| Q | I | Y | T | S | A | V | K | — | S |
| R | I | Y | T | S | A | V | K | — | S |
| Q | S | F | T | S | A | V | K | — | S |
| R | S | F | T | S | A | V | K | — | S |
| Q | I | F | T | S | A | V | K | — | S |
| R | I | F | T | S | A | V | K | — | S |
| Q | S | Y | S | N | T | V | K | — | S |
| R | S | Y | S | N | T | V | K | — | S |
| Q | I | Y | S | N | T | V | K | — | S |
| R | I | Y | S | N | T | V | K | — | S |
| Q | S | F | S | N | T | V | K | — | S |
| R | S | F | S | N | T | V | K | — | S |
| Q | I | F | S | N | T | V | K | — | S |
| R | I | F | S | N | T | V | K | — | S |
| Q | S | Y | T | N | T | V | K | — | S |
| R | S | Y | T | N | T | V | K | — | S |
| Q | I | Y | T | N | T | V | K | — | S |
| R | I | Y | T | N | T | V | K | — | S |
| Q | S | F | T | N | T | V | K | — | S |
| R | S | F | T | N | T | V | K | — | S |
| Q | I | F | T | N | T | V | K | — | S |
| R | I | F | T | N | T | V | K | — | S |
| Q | S | Y | S | S | T | V | K | — | S |
| R | S | Y | S | S | T | V | K | — | S |
| Q | I | Y | S | S | T | V | K | — | S |
| R | I | Y | S | S | T | V | K | — | S |
| Q | S | F | S | S | T | V | K | — | S |
| R | S | F | S | S | T | V | K | — | S |
| Q | I | F | S | S | T | V | K | — | S |
| R | I | F | S | S | T | V | K | — | S |
| Q | S | Y | T | S | T | V | K | — | S |
| R | S | Y | T | S | T | V | K | — | S |
| Q | I | Y | T | S | T | V | K | — | S |
| R | I | Y | T | S | T | V | K | — | S |
| Q | S | F | T | S | T | V | K | — | S |
| R | S | F | T | S | T | V | K | — | S |
| Q | I | F | T | S | T | V | K | — | S |
| R | I | F | T | S | T | V | K | — | S |
| Q | S | Y | S | N | A | A | K | — | S |
| R | S | Y | S | N | A | A | K | — | S |
| Q | I | Y | S | N | A | A | K | — | S |
| R | I | Y | S | N | A | A | K | — | S |
| Q | S | F | S | N | A | A | K | — | S |
| R | S | F | S | N | A | A | K | — | S |
| Q | I | F | S | N | A | A | K | — | S |
| R | I | F | S | N | A | A | K | — | S |
| Q | S | Y | T | N | A | A | K | — | S |
| R | S | Y | T | N | A | A | K | — | S |
| Q | I | Y | T | N | A | A | K | — | S |
| R | I | Y | T | N | A | A | K | — | S |
| Q | S | F | T | N | A | A | K | — | S |
| R | S | F | T | N | A | A | K | — | S |
| Q | I | F | T | N | A | A | K | — | S |
| R | I | F | T | N | A | A | K | — | S |
| Q | S | Y | S | S | A | A | K | — | S |
| R | S | Y | S | S | A | A | K | — | S |
| Q | I | Y | S | S | A | A | K | — | S |
| R | I | Y | S | S | A | A | K | — | S |
| Q | S | F | S | S | A | A | K | — | S |
| R | S | F | S | S | A | A | K | — | S |
| Q | I | F | S | S | A | A | K | — | S |
| R | I | F | S | S | A | A | K | — | S |
| Q | S | Y | T | S | A | A | K | — | S |
| R | S | Y | T | S | A | A | K | — | S |
| Q | I | Y | T | S | A | A | K | — | S |
| R | I | Y | T | S | A | A | K | — | S |
| Q | S | F | T | S | A | A | K | — | S |
| R | S | F | T | S | A | A | K | — | S |
| Q | I | F | T | S | A | A | K | — | S |
| R | I | F | T | S | A | A | K | — | S |
| Q | S | Y | S | N | T | A | K | — | S |
| R | S | Y | S | N | T | A | K | — | S |
| Q | I | Y | S | N | T | A | K | — | S |
| R | I | Y | S | N | T | A | K | — | S |
| Q | S | F | S | N | T | A | K | — | S |
| R | S | F | S | N | T | A | K | — | S |
| Q | I | F | S | N | T | A | K | — | S |
| R | I | F | S | N | T | A | K | — | S |
| Q | S | Y | T | N | T | A | K | — | S |
| R | S | Y | T | N | T | A | K | — | S |
| Q | I | Y | T | N | T | A | K | — | S |
| R | I | Y | T | N | T | A | K | — | S |
| Q | S | F | T | N | T | A | K | — | S |
| R | S | F | T | N | T | A | K | — | S |
| Q | I | F | T | N | T | A | K | — | S |
| R | I | F | T | N | T | A | K | — | S |
| Q | S | Y | S | S | T | A | K | — | S |
| R | S | Y | S | S | T | A | K | — | S |
| Q | I | Y | S | S | T | A | K | — | S |
| R | I | Y | S | S | T | A | K | — | S |
| Q | S | F | S | S | T | A | K | — | S |
| R | S | F | S | S | T | A | K | — | S |
| Q | I | F | S | S | T | A | K | — | S |
| R | I | F | S | S | T | A | K | — | S |
| Q | S | Y | T | S | T | A | K | — | S |
| R | S | Y | T | S | T | A | K | — | S |
| Q | I | Y | T | S | T | A | K | — | S |
| R | I | Y | T | S | T | A | K | — | S |
| Q | S | F | T | S | T | A | K | — | S |
| R | S | F | T | S | T | A | K | — | S |
| Q | I | F | T | S | T | A | K | — | S |
| R | I | F | T | S | T | A | K | — | S |
| Q | S | Y | S | N | A | V | — | G | S |
| R | S | Y | S | N | A | V | — | G | S |
| Q | I | Y | S | N | A | V | — | G | S |
| R | I | Y | S | N | A | V | — | G | S |
| Q | S | F | S | N | A | V | — | G | S |
| R | S | F | S | N | A | V | — | G | S |
| Q | I | F | S | N | A | V | — | G | S |
| R | I | F | S | N | A | V | — | G | S |
| Q | S | Y | T | N | A | V | — | G | S |
| R | S | Y | T | N | A | V | — | G | S |
| Q | I | Y | T | N | A | V | — | G | S |
| R | I | Y | T | N | A | V | — | G | S |
| Q | S | F | T | N | A | V | — | G | S |
| R | S | F | T | N | A | V | — | G | S |
| Q | I | F | T | N | A | V | — | G | S |
| R | I | F | T | N | A | V | — | G | S |
| Q | S | Y | S | S | A | V | — | G | S |
| R | S | Y | S | S | A | V | — | G | S |
| Q | I | Y | S | S | A | V | — | G | S |
| R | I | Y | S | S | A | V | — | G | S |
| Q | S | F | S | S | A | V | — | G | S |
| R | S | F | S | S | A | V | — | G | S |
| Q | I | F | S | S | A | V | — | G | S |
| R | I | F | S | S | A | V | — | G | S |
| Q | S | Y | T | S | A | V | — | G | S |
| R | S | Y | T | S | A | V | — | G | S |
| Q | I | Y | T | S | A | V | — | G | S |
| R | I | Y | T | S | A | V | — | G | S |
| Q | S | F | T | S | A | V | — | G | S |
| R | S | F | T | S | A | V | — | G | S |
| Q | I | F | T | S | A | V | — | G | S |
| R | I | F | T | S | A | V | — | G | S |
| Q | S | Y | S | N | T | V | — | G | S |
| R | S | Y | S | N | T | V | — | G | S |
| Q | I | Y | S | N | T | V | — | G | S |
| R | I | Y | S | N | T | V | — | G | S |
| Q | S | F | S | N | T | V | — | G | S |
| R | S | F | S | N | T | V | — | G | S |
| Q | I | F | S | N | T | V | — | G | S |
| R | I | F | S | N | T | V | — | G | S |
| Q | S | Y | T | N | T | V | — | G | S |
| R | S | Y | T | N | T | V | — | G | S |
| Q | I | Y | T | N | T | V | — | G | S |
| R | I | Y | T | N | T | V | — | G | S |
| Q | S | F | T | N | T | V | — | G | S |
| R | S | F | T | N | T | V | — | G | S |
| Q | I | F | T | N | T | V | — | G | S |
| R | I | F | T | N | T | V | — | G | S |
| Q | S | Y | S | S | T | V | — | G | S |
| R | S | Y | S | S | T | V | — | G | S |
| Q | I | Y | S | S | T | V | — | G | S |
| R | I | Y | S | S | T | V | — | G | S |
| Q | S | F | S | S | T | V | — | G | S |
| R | S | F | S | S | T | V | — | G | S |
| Q | I | F | S | S | T | V | — | G | S |
| R | I | F | S | S | T | V | — | G | S |
| Q | S | Y | T | S | T | V | — | G | S |
| R | S | Y | T | S | T | V | — | G | S |
| Q | I | Y | T | S | T | V | — | G | S |
| R | I | Y | T | S | T | V | — | G | S |
| Q | S | F | T | S | T | V | — | G | S |
| R | S | F | T | S | T | V | — | G | S |
| Q | I | F | T | S | T | V | — | G | S |
| R | I | F | T | S | T | V | — | G | S |
| Q | S | Y | S | N | A | A | — | G | S |
| R | S | Y | S | N | A | A | — | G | S |
| Q | I | Y | S | N | A | A | — | G | S |
| R | I | Y | S | N | A | A | — | G | S |
| Q | S | F | S | N | A | A | — | G | S |
| R | S | F | S | N | A | A | — | G | S |
| Q | I | F | S | N | A | A | — | G | S |
| R | I | F | S | N | A | A | — | G | S |
| Q | S | Y | T | N | A | A | — | G | S |
| R | S | Y | T | N | A | A | — | G | S |
| Q | I | Y | T | N | A | A | — | G | S |
| R | I | Y | T | N | A | A | — | G | S |
| Q | S | F | T | N | A | A | — | G | S |
| R | S | F | T | N | A | A | — | G | S |
| Q | I | F | T | N | A | A | — | G | S |
| R | I | F | T | N | A | A | — | G | S |
| Q | S | Y | S | S | A | A | — | G | S |
| R | S | Y | S | S | A | A | — | G | S |
| Q | I | Y | S | S | A | A | — | G | S |
| R | I | Y | S | S | A | A | — | G | S |
| Q | S | F | S | S | A | A | — | G | S |
| R | S | F | S | S | A | A | — | G | S |
| Q | I | F | S | S | A | A | — | G | S |
| R | I | F | S | S | A | A | — | G | S |
| Q | S | Y | T | S | A | A | — | G | S |
| R | S | Y | T | S | A | A | — | G | S |
| Q | I | Y | T | S | A | A | — | G | S |
| R | I | Y | T | S | A | A | — | G | S |
| Q | S | F | T | S | A | A | — | G | S |
| R | S | F | T | S | A | A | — | G | S |
| Q | I | F | T | S | A | A | — | G | S |
| R | I | F | T | S | A | A | — | G | S |
| Q | S | Y | S | N | T | A | — | G | S |
| R | S | Y | S | N | T | A | — | G | S |
| Q | I | Y | S | N | T | A | — | G | S |
| R | I | Y | S | N | T | A | — | G | S |
| Q | S | F | S | N | T | A | — | G | S |
| R | S | F | S | N | T | A | — | G | S |
| Q | I | F | S | N | T | A | — | G | S |
| R | I | F | S | N | T | A | — | G | S |
| Q | S | Y | T | N | T | A | — | G | S |
| R | S | Y | T | N | T | A | — | G | S |
| Q | I | Y | T | N | T | A | — | G | S |
| R | I | Y | T | N | T | A | — | G | S |
| Q | S | F | T | N | T | A | — | G | S |
| R | S | F | T | N | T | A | — | G | S |
| Q | I | F | T | N | T | A | — | G | S |
| R | I | F | T | N | T | A | — | G | S |
| Q | S | Y | S | S | T | A | — | G | S |
| R | S | Y | S | S | T | A | — | G | S |
| Q | I | Y | S | S | T | A | — | G | S |
| R | I | Y | S | S | T | A | — | G | S |
| Q | S | F | S | S | T | A | — | G | S |
| R | S | F | S | S | T | A | — | G | S |
| Q | I | F | S | S | T | A | — | G | S |
| R | I | F | S | S | T | A | — | G | S |
| Q | S | Y | T | S | T | A | — | G | S |
| R | S | Y | T | S | T | A | — | G | S |
| Q | I | Y | T | S | T | A | — | G | S |
| R | I | Y | T | S | T | A | — | G | S |
| Q | S | F | T | S | T | A | — | G | S |
| R | S | F | T | S | T | A | — | G | S |
| Q | I | F | T | S | T | A | — | G | S |
| R | I | F | T | S | T | A | — | G | S |
| Q | S | Y | S | N | A | V | K | G | S |
| R | S | Y | S | N | A | V | K | G | S |
| Q | I | Y | S | N | A | V | K | G | S |
| R | I | Y | S | N | A | V | K | G | S |
| Q | S | F | S | N | A | V | K | G | S |
| R | S | F | S | N | A | V | K | G | S |
| Q | I | F | S | N | A | V | K | G | S |
| R | I | F | S | N | A | V | K | G | S |
| Q | S | Y | T | N | A | V | K | G | S |
| R | S | Y | T | N | A | V | K | G | S |
| Q | I | Y | T | N | A | V | K | G | S |
| R | I | Y | T | N | A | V | K | G | S |
| Q | S | F | T | N | A | V | K | G | S |
| R | S | F | T | N | A | V | K | G | S |
| Q | I | F | T | N | A | V | K | G | S |
| R | I | F | T | N | A | V | K | G | S |
| Q | S | Y | S | S | A | V | K | G | S |
| R | S | Y | S | S | A | V | K | G | S |
| Q | I | Y | S | S | A | V | K | G | S |
| R | I | Y | S | S | A | V | K | G | S |
| Q | S | F | S | S | A | V | K | G | S |
| R | S | F | S | S | A | V | K | G | S |
| Q | I | F | S | S | A | V | K | G | S |
| R | I | F | S | S | A | V | K | G | S |
| Q | S | Y | T | S | A | V | K | G | S |
| R | S | Y | T | S | A | V | K | G | S |
| Q | I | Y | T | S | A | V | K | G | S |
| R | I | Y | T | S | A | V | K | G | S |
| Q | S | F | T | S | A | V | K | G | S |
| R | S | F | T | S | A | V | K | G | S |
| Q | I | F | T | S | A | V | K | G | S |
| R | I | F | T | S | A | V | K | G | S |
| Q | S | Y | S | N | T | V | K | G | S |
| R | S | Y | S | N | T | V | K | G | S |
| Q | I | Y | S | N | T | V | K | G | S |
| R | I | Y | S | N | T | V | K | G | S |
| Q | S | F | S | N | T | V | K | G | S |
| R | S | F | S | N | T | V | K | G | S |
| Q | I | F | S | N | T | V | K | G | S |
| R | I | F | S | N | T | V | K | G | S |
| Q | S | Y | T | N | T | V | K | G | S |
| R | S | Y | T | N | T | V | K | G | S |
| Q | I | Y | T | N | T | V | K | G | S |
| R | I | Y | T | N | T | V | K | G | S |
| Q | S | F | T | N | T | V | K | G | S |
| R | S | F | T | N | T | V | K | G | S |
| Q | I | F | T | N | T | V | K | G | S |
| R | I | F | T | N | T | V | K | G | S |
| Q | S | Y | S | S | T | V | K | G | S |
| R | S | Y | S | S | T | V | K | G | S |
| Q | I | Y | S | S | T | V | K | G | S |
| R | I | Y | S | S | T | V | K | G | S |
| Q | S | F | S | S | T | V | K | G | S |
| R | S | F | S | S | T | V | K | G | S |
| Q | I | F | S | S | T | V | K | G | S |
| R | I | F | S | S | T | V | K | G | S |
| Q | S | Y | T | S | T | V | K | G | S |
| R | S | Y | T | S | T | V | K | G | S |
| Q | I | Y | T | S | T | V | K | G | S |
| R | I | Y | T | S | T | V | K | G | S |
| Q | S | F | T | S | T | V | K | G | S |
| R | S | F | T | S | T | V | K | G | S |
| Q | I | F | T | S | T | V | K | G | S |
| R | I | F | T | S | T | V | K | G | S |
| Q | S | Y | S | N | A | A | K | G | S |
| R | S | Y | S | N | A | A | K | G | S |
| Q | I | Y | S | N | A | A | K | G | S |
| R | I | Y | S | N | A | A | K | G | S |
| Q | S | F | S | N | A | A | K | G | S |
| R | S | F | S | N | A | A | K | G | S |
| Q | I | F | S | N | A | A | K | G | S |
| R | I | F | S | N | A | A | K | G | S |
| Q | S | Y | T | N | A | A | K | G | S |
| R | S | Y | T | N | A | A | K | G | S |
| Q | I | Y | T | N | A | A | K | G | S |
| R | I | Y | T | N | A | A | K | G | S |
| Q | S | F | T | N | A | A | K | G | S |
| R | S | F | T | N | A | A | K | G | S |
| Q | I | F | T | N | A | A | K | G | S |
| R | I | F | T | N | A | A | K | G | S |
| Q | S | Y | S | S | A | A | K | G | S |
| R | S | Y | S | S | A | A | K | G | S |
| Q | I | Y | S | S | A | A | K | G | S |
| R | I | Y | S | S | A | A | K | G | S |
| Q | S | F | S | S | A | A | K | G | S |
| R | S | F | S | S | A | A | K | G | S |
| Q | I | F | S | S | A | A | K | G | S |
| R | I | F | S | S | A | A | K | G | S |
| Q | S | Y | T | S | A | A | K | G | S |
| R | S | Y | T | S | A | A | K | G | S |
| Q | I | Y | T | S | A | A | K | G | S |
| R | I | Y | T | S | A | A | K | G | S |
| Q | S | F | T | S | A | A | K | G | S |
| R | S | F | T | S | A | A | K | G | S |
| Q | I | F | T | S | A | A | K | G | S |
| R | I | F | T | S | A | A | K | G | S |
| Q | S | Y | S | N | T | A | K | G | S |
| R | S | Y | S | N | T | A | K | G | S |
| Q | I | Y | S | N | T | A | K | G | S |
| R | I | Y | S | N | T | A | K | G | S |
| Q | S | F | S | N | T | A | K | G | S |
| R | S | F | S | N | T | A | K | G | S |
| Q | I | F | S | N | T | A | K | G | S |
| R | I | F | S | N | T | A | K | G | S |
| Q | S | Y | T | N | T | A | K | G | S |
| R | S | Y | T | N | T | A | K | G | S |
| Q | I | Y | T | N | T | A | K | G | S |
| R | I | Y | T | N | T | A | K | G | S |
| Q | S | F | T | N | T | A | K | G | S |
| R | S | F | T | N | T | A | K | G | S |
| Q | I | F | T | N | T | A | K | G | S |
| R | I | F | T | N | T | A | K | G | S |
| Q | S | Y | S | S | T | A | K | G | S |
| R | S | Y | S | S | T | A | K | G | S |
| Q | I | Y | S | S | T | A | K | G | S |
| R | I | Y | S | S | T | A | K | G | S |
| Q | S | F | S | S | T | A | K | G | S |
| R | S | F | S | S | T | A | K | G | S |
| Q | I | F | S | S | T | A | K | G | S |
| R | I | F | S | S | T | A | K | G | S |
| Q | S | Y | T | S | T | A | K | G | S |
| R | S | Y | T | S | T | A | K | G | S |
| Q | I | Y | T | S | T | A | K | G | S |
| R | I | Y | T | S | T | A | K | G | S |
| Q | S | F | T | S | T | A | K | G | S |
| R | S | F | T | S | T | A | K | G | S |
| Q | I | F | T | S | T | A | K | G | S |
| R | I | F | T | S | T | A | K | G | S |

“—” indicates the absence of a residue at that positon with reference to SEQ ID NO: 22

TABLE 12
Heavy chain analysis

| | SEQ | | | | | | | | | | |
|---|---|---|---|---|---|---|---|---|---|---|---|
| Chain | ID | | | | | | | | | | |
| Name | NO: | V | D | J | FR1 | CDR1 | FR2 | CDR2 | FR3 | CDR3 | FR4 |
| | 57 | Germline | | | QVQLQESG | SGGYYWS | WIRQHPG | YIYYSGS | RVTISVDTSKNQ | VVPAA-- | WGQ |
| | | | | | PGLVKPSQ | | KGLEWIG | TYYNPSL | FSLKLSSVTAAD | YYYYYGM | GTT |
| | | | | | TLSLTCTV | | | KS | TAVYYCAR | DV | VTV |
| | | | | | SGGSIS | | | | | | SS |
| 3G11.1A6 | 2 | VH4-31 | D2-2 | JH6B | RVQLQESG | SGGYFWT | WIRQHPG | YIYYSGS | RVTISVDTSKNQ | AVPAAKG | WGQ |
| | | | | | PGLVKPTQ | | KGLEWIG | TYYSPSL | FSLKLSSVTAAD | YYSYYGM | GTT |
| | | | | | TLSLTCTV | | | KS | TAVYYCTR | DV | VTV |
| | | | | | SGSSII | | | | | | SS |
| 2E10.1B9 | 6 | VH4-31 | D2-2 | JH6B | RVQLQESG | SGGYFWT | WIRQHPG | YIYYSGS | RVTISVDTSKNQ | AVPAAKG | WGQ |
| | | | | | PGLVKPTQ | | KGLEWIG | TYYSPSL | FSLKLSSVTAAD | YYSYYGM | GTT |
| | | | | | TLSLTCTV | | | KS | TAVYYCTR | DV | VTV |
| | | | | | SGGSII | | | | | | SS |
| 2A9.1A1 | 10 | VH4-31 | D2-2 | JH6B | RVQLQESG | SGGYFWT | WIRQHPG | YIYYSGS | RVTISVDTSKNQ | AVPAAKG | WGQ |
| | | | | | PGLVKPTQ | | KGLEWIG | TYYSPSL | FSLKLSSVTAAD | YYSYYGM | GTT |
| | | | | | TLSLTCTV | | | KS | TAVYYCTR | DV | VTV |
| | | | | | SGGSII | | | | | | SS |
| 2C5.2B12 | 14 | VH4-31 | D2-2 | JH6B | RVQLQESG | SGGYFWT | WIRQHPG | YIYYSGS | RVTISVDTSKNQ | AVPAAKG | WGQ |
| | | | | | PGLVKPTQ | | KGLEWIG | TYYSPSL | FSLKLSSVTAAD | YYSYYGM | GTT |
| | | | | | TLSLTCTV | | | KS | TAVYYCTR | DV | VTV |
| | | | | | SGGSII | | | | | | SS |
| 3C2.2A8 | 18 | VH4-31 | D2-2 | JH6B | RVQLQESG | SGGYFWT | WIRQHPG | YIYYSGS | RVTISVDTSKNQ | AVPAAKG | WGQ |
| | | | | | PGLVKPTQ | | KGLEWIG | TYYSPSL | FSLKLSSVTAAD | YYSYYGM | GTT |
| | | | | | TLSLTCTV | | | KS | TAVYYCTR | DV | VTV |
| | | | | | SGGSII | | | | | | SS |
| 3C5 | 22 | VH4-31 | D2-2 | JH6B | RVQLQESG | SGGYFWT | WIRQHPG | YIYYSGS | RVTISVDTSKNQ | AVPAAKG | WGQ |
| | | | | | PGLVKPSQ | | KGLEWIG | TYYSPSL | FSLKLSSVTAAD | YYSYYGM | GTT |
| | | | | | TLSLTCTV | | | KS | TAVYYCTR | DV | VTV |
| | | | | | SGGSII | | | | | | SS |
| | 58 | Germline | | | QVQLVQSG | GYYMH | WVRQAPG | WINPNSG | RVTMTRDTSIST | -- | WGQ |
| | | | | | AEVKKPGA | | QGLEWMG | GTNYAQK | AYMELSRLRSDD | GYSSGWY | GTL |
| | | | | | SVKVSCKA | | | FQG | TAVYYCAR | DY | VTV |
| | | | | | SGYTFT | | | | | | SS |
| 8A6.1A3 | 32 | VH1-2 | D6-19 | JH4B | QVQLVQSG | GYYMH | WVRQAPG | WINSNSG | RVTMTRDTSIST | GRGYSSG | WGQ |
| | | | | | AEVKKPGA | | QGLEWMG | GTNYAQK | AYMELSRLRSDD | WYDY | GIL |
| | | | | | SVKVSCKA | | | FQG | TAVYYCAR | | VTV |
| | | | | | SGYTFT | | | | | | SS |
| 9E2.3A8 | 36 | VH1-2 | D6-19 | JH4B | QVQLVQSG | GYYMH | WVRQAPG | WINPNSG | RVTMTRDTSIST | GRGYSSG | WGQ |
| | | | | | VEVKKPGA | | QGLEWMG | GTNSAQK | AYMELSRLRSDD | WYDY | GTL |
| | | | | | SVKVSCKA | | | FQG | TAVYYCAR | | VTV |
| | | | | | SGYTFT | | | | | | SS |
| | 59 | Germline | | | QVQLVQSG | GYYMH | WVRQAPG | WINPNSG | RVTMTRDTSIST | GRGYSSG | WGQ |
| | | | | | AEVKKPGA | | QGLEWMG | GTNYAQK | AYMELSRLRSDD | WYDY | GTL |
| | | | | | SVKVSCKA | | | FQG | TAVYYCAR | | VTV |
| | | | | | SGYTFT | | | | | | SS |
| 2F5.1A4 | 40 | VH1-2 | D6-19 | JH5B | QMQLVQSG | GYYMH | WVRQAPG | WINTNSG | RVTMTRDTSIST | GRGYSSG | WGQ |
| | | | | | AEVKKPGA | | QGLEWMG | GTNCAQK | AYMELSRLRSDD | WYDY | GTL |
| | | | | | SVKVSCKA | | | FQV | TAVYYCAR | | VTV |
| | | | | | SGYTFT | | | | | | SS |
| | 60 | Germline | | | QVQLVESG | SYGMH | WVRQAPG | VIWYDGS | RFTISRDNSKNT | DYGG- | WGQ |
| | | | | | GGVVQPGR | | KGLEWVA | NKYYADS | LYLQMNSLRAED | YFDY | GTL |
| | | | | | SLRLSCAA | | | VKG | TAVYYCAR | | VTV |
| | | | | | SGFTFS | | | | | | SS |
| 7B2.3B10 | 44 | VH3-33 | D4-23 | JH4B | QVQLVESG | SYGMH | WVRQAPG | VIWYDGS | RFTISRDNSKNT | DKGGHYF | WGQ |
| | | | | | GGVVQPGR | | KGLEWVA | YKYYADS | LYLQMNSLRAED | DY | GTL |
| | | | | | SLRLSCAA | | | VQG | TAVYYCAR | | VTV |
| | | | | | SGFTFS | | | | | | SS |
| | 61 | Germline | | | QVQLVESG | SYGMH | WVRQAPG | VIWYDGS | RFTISRDNSKNT | --- | WGQ |
| | | | | | GGVVQPGR | | KGLEWVA | NKYYADS | LYLQMNSLRAED | YSSSWFD | GTL |
| | | | | | SLRLSCAA | | | VKG | TAVYYCAR | Y | VTV |
| | | | | | SGFTFS | | | | | | SS |
| 5B11 | 48 | VH3-33 | D6-13 | JH4B | QVQLVESG | SYGMH | WVRQTPG | IIWYDGG | RFTISRDNSKNT | DHRYGSS | WGQ |
| | | | | | GGVVQPGR | | KGLEWVA | IEYYADS | LYLQMNSLRAED | WFDY | GTL |
| | | | | | SVRLSCAA | | | VKG | TAVYYCAR | | VTV |
| | | | | | SGFTFS | | | | | | SS |

TABLE 13
Light chain analysis

| | SEQ | | | | | | | | | |
|---|---|---|---|---|---|---|---|---|---|---|
| Chain | ID | | | | | | | | | |
| Name | NO: | V | J | FR1 | CDR1 | FR2 | CDR2 | FR3 | CDR3 | FR4 |
| | 62 | Germline | | DIVMTQSPLSLP | RSSQSLLHS | WYLQKPGQ | LGSN | GVPDRFSGSGSGTDFT | MQALQ | FGPGT |
| | | | | VTPGEPASISC | NGYNYLD | SPQLLIY | RAS | LKISRVEAEDVGVYYC | TPFT | KVDIK |
| 3G11.1A6 | 4 | A3 | JK3 | DIVMTQSPLSLP | RSSQSLLHS | WFLQKPGQ | LGSN | GVPDRFSGSGSGTDFT | MQALQ | FGPGT |
| | | | | VTPGEPASISC | NGYNYLD | SPQLLIY | RAS | LKISRVEAEDVGVYYC | TPFT | KVDIK |
| 2E10.1B9 | 8 | A3 | JK3 | DIVMTQSPLSLP | RSSQSLLHS | WFLQKPGQ | LGSN | GVPDRFSGSGSGTDFT | MQALQ | FGPGT |
| | | | | VTPGEPASISC | NGYNYLD | SPQLLIY | RAS | LKISRVEAEDVGVYYC | TPFT | KVDIK |
| 2A9.1A1 | 12 | A3 | JK3 | DIVMTQSPLSLP | RSSQSLLHS | WFLQKPGQ | LGSN | GVPDRFSGSGSGTDYT | MQALQ | FGPGT |
| | | | | VTPGEPASISC | NGYNYLD | SPQLLIY | RAS | LKISRVEAEDVGVYYC | TPFT | KVDIK |
| 2C5.2B12 | 16 | A3 | JK3 | DIVMTQSPLSLP | RSSQSLLHS | WFLHKPGQ | LGSN | GVPDRFSGSGSGTDFT | MQALQ | FGPGT |
| | | | | VTPGEPASISC | NGYNYLD | SPQLLIY | RAS | LKISRVEAEDVGVYYC | TPFT | KVDIK |
| 3C2.2A8 | 20 | A3 | JK3 | DIVMTQSPLSLP | RSSQSLLHS | WFLHKPGQ | LGSN | GVPDRFSGSGSGTDFT | MQALQ | FGPGT |
| | | | | VTPGEPASISC | NGYNYLD | SPQLLIY | RAS | LKISRVEAEDVGVYYC | TPFT | KVDIK |
| 3C5 | 24 | A3 | JK3 | DIVMTQSPLSLP | RSSQSLLHS | WFLQKPGQ | LSSN | GVPDRFSGSGSGTDFT | MQALQ | FGPGT |
| | | | | VTPGEPASISC | SGYNYLD | SPQLLIY | RAS | LKISRVEAEDVGVYYC | TPFT | KVDIK |
| | 63 | Germline | | DIVMTQSPDSLA | KSSQSVLYS | WYQQKPGQ | WAST | GVPDRFSGSGSGTDFT | QQYYN | FGQGT |
| | | | | VSLGERATINC | SNNKNYLA | PPKLLIY | RES | LTISSLQAEDVAVYYC | IPWT | KVEIK |
| 8A6.1A3 | 34 | B3 | JK1 | DIVMTQSPDSLA | KSSQSVFFS | WYQQKPGQ | WAST | GVPDRFSGSGSGTDFT | QQYYN | FGQGT |
| | | | | VSLGERATINC | SSNENYLT | PPKLLIY | RES | LTISSLQAEDVAVYYC | IPWT | KVEIK |
| 9E2.3A8 | 38 | B3 | JK1 | DIVMTQSPDSLA | KSSQSVLHN | WYQQKPGQ | WASS | GVPDRFSGSGSGTDFT | QQYYN | FGQGT |
| | | | | VSLGERATINC | SHNENYLT | PPKLLIY | RES | LSISSLQADDVAVYYC | IPWT | KVEIK |
| 2F5.1A4 | 42 | B3 | JK1 | DIVMTQSPDSLA | KSNQNILYS | WYQQKPGQ | REWA | GVPDRFSGSGSGTDFT | QQYYN | FGQGT |
| | | | | VSLGERATINC | SSNENYLA | PPKLLIY | STS | LTISSLQAEDVAVYYC | IPWT | KVEIK |
| | 64 | Germline | | DIQMTQSPSSLS | RASQSISSY | WYQQKPGK | AASS | GVPSRFSGSGSGTDFT | QQSFS | FGQGT |
| | | | | ASVGDRVTITC | LN | APKLLIY | LQS | LTISSLQPEDFATYYC | FPPEW | KVEIK |
| | | | | | | | | | T | |
| 7B2.3B10 | 46 | O2 | JK1 | DIQMTQSPSSLS | RASQSISSY | WYQQKPGK | AASS | GVPSRFSGSGSGTDFT | QQSFS | FGQGT |
| | | | | ASVGDRVTITC | LN | APKLLIY | LQS | LTISSLQPEDFATYYC | FPPEW | KVEIK |
| | | | | | | | | | T | |
| | 65 | Germline | | DIQMTQSPSSLS | RASQGISNY | WFQQKPGK | AASS | GVPSRFSGSGSGTDFT | QQYKS | FGGGT |
| | | | | ASVGDRVTITC | LA | APKSLIY | LQS | LTISSLQPEDFATYYC | YPLT | KVEIK |
| 5B11 | 50 | L1 | JK4 | DIQMTQSPSSLS | RASQGINNY | WFQQKPGK | AASS | GVPSNFSGSGSGTDFT | QQYKS | FGGGT |
| | | | | ASVGDRVTITC | LA | APKSLIY | LQS | LSISSLQPEDFATYYC | YPLT | KVEIR |

### Example 10

### Potency (IC50) Determination of α5β1 Antibodies Inhibition of α5β1-Mediated K563 Binding to Fibronectin

In order to confirm activity and determine the relative potency of the different cloned purified antibodies, activity in the K562-fibronectin adhesion assay was determined. Plates were coated overnight with 3-5 μg/ml GST-Fibronectin type III domains 9-10, and pre-blocked with 3% BSA/PBS for 1 hour prior to the assay. Cells were then pelleted and washed twice in HBSS, after which the cells were then resuspended in HBSS at 1×10⁶ cells/ml. To select the best antibodies the cells were incubated in the presence of appropriate antibodies at 4° C. for 60 minutes in a V-bottom plate. To increase the stringency of the assay cells the pre-incubation step was removed. The 3% BSA/PBS was removed from the assay plates and the plates washed twice with PBS or HBSS, and the cell-antibody mixtures were transferred to the coated plate and the plate was incubated at 37° C. for 60 minute in the presence of 1 mM MnCl₂. The cells on the coated plates were then washed four times in warm HBSS, and the cells were thereafter frozen at ˜80° C. for one hour. The cells were allowed to thaw at room temperature for one hour, and then 100 μL of CyQuant dye/lysis buffer (Molecular Probes) was added to each well according to the manufacturer's instructions. Fluorescence was read at an excitation wavelength of 485 nm and an emission wavelength of 530 nm. As a standard control the commercial α5β1 neturalising antibody IIA1 (R&D Systems) was included.

TABLE 14
Inhibition of α5β1 mediated adhesion of K562 cells to fibronectin.

| MAb ID | IC50 ng/mL |
|---|---|
| 2F5.1A4 | 97.8 |
| 7D11 | 99.5 |
| 7F9 | 49.8 |
| 3G11.1A6 | 9.0 |
| 8A6.1A3 | 52.2 |
| 9E2.3A8 | 73.4 |
| 7G2 | 55.8 |
| 7B2.3B10 | 57.0 |
| 5B11 | 28.4 |
| 2C7 | 20.1 |
| 4F1 | 27.6 |
| 2A9.1A1 | 14.1 |
| 2C5.2B12 | 11.9 |
| 3C2.2A8 | 12.4 |
| 3C5 | 12.4 |
| IIA1 | 16.68 |

### Example 11

### Cloned Purified Antibodies Exhibit Selective Inhibition of α5 Integrin but not α4 Integrin in j6 Cells

To confirm that the antibodies were specific to α5 or α5β1 (and not binding to β1), the antibodies were also screened in an alpha4beta dependent adhesion assay. For this, the ability of our antibodies to block the binding of J6.77 Jurkat cells to the CS-1 fragment of fibronectin was tested. Plates were coated overnight at 4° C. with 2.5 ug/ml GST-CS-1 fragment of fibronectin in PBS, washed twice in PBS and then and blocked with 3% BSA/PBS for 1 hour. Cells were then pelleted and washed 3 times with 1% BSA/HBSS and resuspended in HBSS at a concentration of 9×10⁵/ml. Cells were dispensed into V bottom pates (35 ul per well), antibody was added at a final concentration of 5 ug/ml in 35 ul HBSS added to each well, and then incubated for 1 hour at 4° C. Assay plates were then washed 3 times with PBS. The mix of cells and antibody was then transferred to the assay plate and incubated for 40 minutes at 37° C. in the presence of 0.2 mM MnCl₂. The cells on the coated plates were then washed four times in warm HBSS, and the cells were thereafter frozen at −80° C. for one hour. The cells were allowed to thaw at room temperature for one hour, and then 100 μL of CyQuant dye/lysis buffer (Molecular Probes) was added to each well according to the manufacturer's instructions. Fluorescence was read at an excitation wavelength of 485 nm and an emission wavelength of 530 nm. The majority of the antibodies showed little to no blockade in this assay, suggesting that their specificity is primarily against α5 or α5β1.

TABLE 15
Inhibition of α4β1 mediated J6 cell adhesion to GST-CS-1.
Inhibition at 5 ug/ml expressed as a percentage.

| | Average % |
|---|---|
| MAb ID | inhibition |
| 2F5.1A4 | −12 |
| 7D11 | −11 |
| 2F1 | −5 |
| 3G11.1A6 | −2 |
| 8A6.1A3 | −23 |
| 9E2.3A8 | 2 |
| 7G2 | −9 |
| 8B2 | −17 |
| 7B2.3B10 | −14 |
| 5B11 | −9 |
| 2C7 | −7 |
| 4F1 | 13 |
| 2A9.1A1 | −19 |
| 2C5.2B12 | 18 |
| 3C2.2A8 | 1 |
| 3C5 | 0 |

### Example 12

### Lead Antibodies do not Inhibit of AVB3 and AVB5 Mediated Adhesion to Osteopontin (OPN) and Vitronectin (VN)

To confirm the purified antibodies did not cross-react or inhibit av integrins the ability of a smaller subset of clones to block A375M (ATCC #CRL 1619) adhesion to vitronectin (VN) and osteopontin (OPN) was assessed. Plates were coated with either 1.25 ug/mL purified human VN (Becton Dickinson) or 313 ng/mL GST OPN aa17-168 in phosphate buffer pH9.0, overnight at 4° C. Plates were then washed and pre-blocked with 3% BSA/PBS for 1 hour prior to the assay.

A375M cells were cultured in DMEM (Hepes modification) with L-Glutamine, sodium pyruvate, and 10% FCS. Cells were trypsinised, pelleted and washed 3× in HBSS, then resuspended in HBSS at appropriate concentration (30000 cells in 35 uL HBSS) and 35 uL of 2× antibody, each antibody was at a final of 5 ug/ml. Cells and antibody were co-incubated for 40 min at 4° C. Assay plates were then washed 3 times with HBSS, the cells and antibody transferred to the assay plate and incubated for 40 minutes at 37° C. The plates were then washed 3 times in warm HBSS. To determine the number of cells bound, plates were placed at −80° C. for minutes, thawed at room temperature and 100 uL of CyQuant dye/lysis buffer to each well as per Molecular Probes procedure. Plates were read Flourescence at 485 nm excitation and 530 emission. As a positive control the commercial av integrin neutralising antibody L230 (Chemicon) was included. Collectively the data confirm that these antibodies are specific for α5β1 integrin over avb3 and avb5 integrins.

TABLE 16
Inhibition of A375M adhesion to vitronectin and osteopontin.
Activity is expressed as % inhibition at 5 ug/ml.

| | Vitronectin | Osteopontin |
|---|---|---|
| | Adhesion | Adhesion |
| MAb ID | (% inhibition) | (% inhibition) |
| 7D11 | 8 | 16 |
| 3G11.1A6 | 12 | 3 |
| 8A6.1A3 | 6 | 7 |
| 9E2.3A8 | −4 | 7 |
| 7G2 | 6 | 8 |
| 5B11 | 9 | 13 |
| 2C7 | 1 | 15 |
| 4F1 | −9 | 7 |
| 2A9.1A1 | 0 | −3 |
| 2C5.2B12 | 16 | 0 |
| 3C2.2A8 | 16 | −3 |
| 3C5 | 10 | 3 |
| L230 | 96 | 97 |

### Example 13

### Inhibition of α5β1 Mediated Adhesion of HUVEC Cells TO Fibronectin

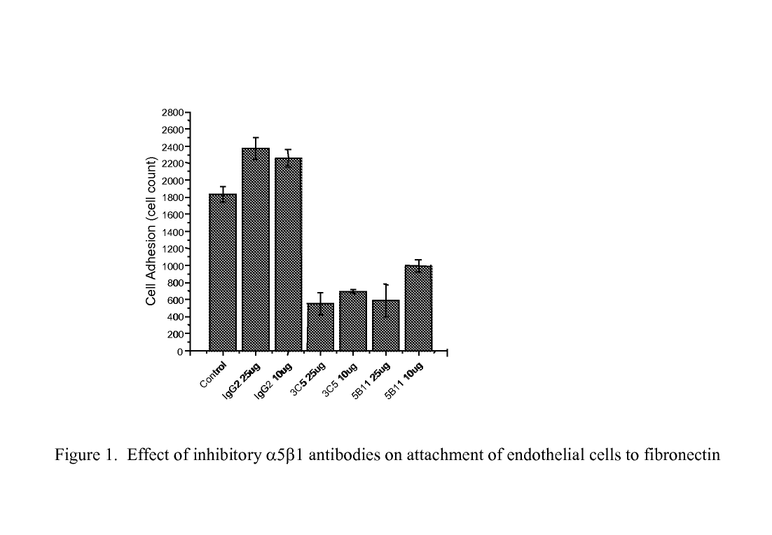

To determine whether the α5β1 blocking antibodies were able to block α5β1, function on HUVECs and adhesion assay was performed. Black clear-bottomed plates were incubated overnight at 4° C. with 100 ul per well of a GST fusion protein fibronectin fragment 9-10 at 10 ug/ml in PBS. The following day plates were washed and blocked with 1% BSA in PBS. The adhesion assay was carried out in MCDB131 media using 25000 HUVEC cells per well. To block av integrins (which also bind to fibronectin) cells were also pre-incubated with the av integrin blocking antibody L230 at 10 ug/ml. The cells were incubated at 37 for 45 minutes, unbound cell were washed away, the adhered cells were fixed and stained with Hoescht. The number of cells adhered were counted on the Arrayscan.
The data in FIG. 1 shows that 3C5 and 5B11 are all effective inhibitors of α5β1 integrin on HUVEC cells.

### Example 14

### Determination of Binding Affinity of Purified Antibodies

To assess the affinity of the antibodies for α5β1, a FACS based KD analysis was employed. As the antibodies do not interact well with recombinant integrin classical Biacore analysis was not considered relevant. K562 cells expressing α5β1 were resuspended in filtered HBS buffer containing 1 mM MgCl₂ and 1 mM CaCl₂ at a concentration of approximately 2.5-6 million cells/mL. The cells were kept on ice. Serially diluted (2×) mAbs across 11 wells in a 96-well plate. All mAbs were diluted in HBS described above. Additional HBS and cells were added to each well so that the final volume was 300 μL/well and each well contained approximately 140,000-150,000 cells. The final molecular concentration range for each mAb was 3C5: 3.8 nM -7.5 μM; 5B11: 3.8 nM-7.3 μM. Cells were incubated on a plate shaker for 5 hours at 4° C. and then were spun/washed 3× at 4° C. with HBS. 250 μL of 99 nM Cy5 goat α-human polyclonal antibody (Jackson ImmunoResearch Laboratories, #109-175-008) was added to each well, and incubated with shaking for 40 minutes at 4° C. The cells were again spun/washed 3× at 4° C. with HBS. The Mean Fluorescence (F) of 7000 “events” was determined using FACS Canto II HTS flow cytometry instrumentation. To calculate affinity, data was fitted nonlinearly to a plot of the Mean Fluorescence as a function of molecular mAb concentration with Scientist 3.0 software using the equation:

$$F = {P}^{′} \frac{( {K}_{D} + {L}_{T} + n \cdot M ) - \sqrt{{( {K}_{D} + {L}_{T} + n \cdot M )}^{2} - 4 n \cdot M \cdot {L}_{T}}}{2} + B$$

In this equation, F=mean fluorescence, L_{T}=total molecular mAb concentration, P′=proportionality constant that relates arbitrary fluorescence units to bound mAb, M=cellular concentration in molarity, n=number of receptors per cell, B=background signal, and K_{D}=equilibrium dissociation constant. For each mAb titration curve an estimate for K_{D} is obtained as P′, n, B, and K_{D} are allowed to float freely in the nonlinear analysis. (A. W. Drake and S. L. Klakamp. A rigorous multiple independent binding site model for determining cell-based equilibrium dissociation constants. J. Immunol. Methods, 2007, Vol. 318, 157-162.)
Each plot with the nonlinear fit (green line) is shown below. The table lists the resulting K_{D} for each mAb in order of decreasing affinity along with the 95% confidence interval of the fit. The nonlinear fit for each titration curve with the 4-parameter model was able to return reasonable 95% confidence intervals for K_{D}. This implies each curve possesses some K_{D} influence and hence the returned values for K_{D} are most likely acceptable estimates of either the stoichiometric or site-binding dissociation constant.

TABLE 17
Affinity of 3C5 and 5B11 for cellular α5β1

| MAb ID | K_{D} (pM) | 95% CI (pM) |
|---|---|---|
| 3C5 | 14.7 | ±8.3 |
| 5B11 | 34.7 | ±15.2 |

### Example 15

### 3C5 and 5B11 Inhibit Angiogenesis in an In Vitro Co-Culture Model of Endothelial Tube Formation

α5β1 integrin is thought to play a role in regulating the formation of new blood vessels. To understand whether the antibodies have the potential to modulate endothelial cell function, the ability of the antibodies to impact endothelial tube formation were assessed using an in vitro co-culture assay and an in vivo angiogenesis assay.

### In Vitro Co-Culture Assay:

Endothelial cells are cultured on a monolayer or feeder layer of dermal fibroblast, and over a period of 11 days network of endothelial cell tubes is established. The co-culture kit was purchased from TCS Biologicals (UK) and performed as per manufacturers instructions. Antibodies were dosed at the concentrations indication and the media changed every 2-3 days. Tubes were visualised by staining for PECAM/CD31 as per manufacturers instructions, and quantitated using the neurite outgrowth algorithm on a KS400.

### Example 16

### Determination of In Vivo Efficacy of Purified Antibodies: Evaluation of the Antiangiogenic Efficacy on a Spheroid-Based In Vivo Angiogenesis Assay

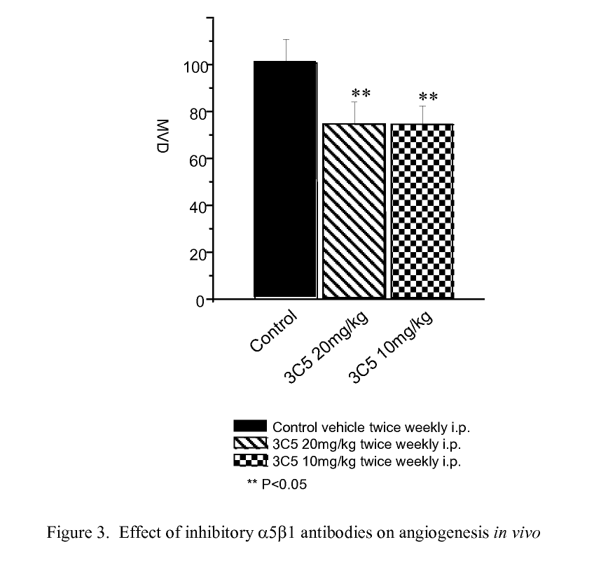

As the antibodies do not cross react with mouse integrin, the impact of the antibodies targeting α5β1 on vessel formation was assessed in a matrigel plug seeded with human endothelial cells. In this model the human endothelial cells form functional angiogenic vessels allowing the therapeutic impact of the antibodies to be studied. Human umbilical vein endothelial cell (HUVEC) spheroids were prepared as described earlier (Korff and Augustin: J Cell Biol 143: 1341-52, 1998) by pipetting 100 endothelial cells (EC) in a hanging drop on plastic dishes to allow overnight spheroid formation. The following day, using the method previously described (Alajati et al: Nature Methods 5:439-445, 2008), EC spheroids were harvested and mixed in a Matrigel/fibrin solution with single HUVECs to reach a final number of 100,000 ECs as spheroids and 200,000 single ECs per injected plug. VEGF-A and FGF-2 were added at a final concentration of 1000 ng/ml. Human umbilical vein endothelial cell (HUVEC) spheroids (1000 spheroids; 100 cells/spheroid) and HUVECs in suspension (200,000 cells) were injected subcutaneously into the flank of a SCID mouse in a Matrigel/fibrin matrix containing VEGF-A/FGF-2 (each 1000 ng/ml). The following day (day 1) treatment commenced. At day 21 the study was terminated. The matrix plugs were removed and fixed in 4% PFA. All matrix plugs were paraffin embedded and cut to a thickness of 8-10 μm section for histological examination. Blood vessels were visualized by staining for human CD34 and smooth muscle actin (SMA) and the microvessel density (MVD) and pericyte coverage was determined.
As illustrated in FIG. 3, MAb 3C5 is effective in inhibiting vessel formation in vivo.

### Example 17

### 3C5 Inhibits Growth of U87MG and A549 Tumours by Targeting Both the Tumour Cells and Host Cells

α5β1 plays a role in regulating the function of a number of different cell types including endothelial cells and tumour cells. To explore the direct effects of targeting α5β1 inhibition in the tumour cell compartment in vivo we exploited the fact that 3C5 does not cross react with murine integrin, and assessed impact of growth on human xenografts. U87-MG and MDA-MB-231 tumours were established by s.c injection of 2.5×10⁶ cells alone and 1×10⁷ cells in 50% matrigel respectively, into the hind flank of nude (nu/nu genotype) mice. Dosing, 20 mg/kg i.p. twice weekly, commenced when tumours were established.

In order to model the impact of inhibiting α5β1 simultaneously in both the host and tumour a strain of SCID mice were bred in which the murine alpha5 chain was replaced with the human alpha5 chain. The impact of 3C5 on the growth of A549 in the human α5 expressing SCID animals was assessed. Transgenic mice expressing human α5 were implanted with A549 tumours, established by s.c. injection of 2×10⁶ cells (without matrigel) into the hind flank of SCID (α5β1 ko/ki/SCID (hα5β1-SCID)) mice. Dosing, 20 mg/kg i.p. twice weekly, commenced when tumours were established.

The data (Table 18) demonstrate that 3C5 impacts tumour growth either directly by targeting the tumour, or indirectly by targeting the host.

TABLE 18
Inhibition of tumour growth by 3C5

| | | % Inhibition |
|---|---|---|
| | | Geometric Mean |
| MAb ID | Model | Delta Volume |
| 3C5 | U87-MG (nude) | 29* |
| | A549 (ha5 SCID) | 56* |
| | A549 (ha5 SCID) | 63** |

***p < 0.001;
**p < 0.01;
*p < 0.05;
NS—not significant

### Example 18

### Inhibition of Tumour Cell Growth in Human Patients

A group of human cancer patients diagnosed with pancreatic cancer is randomized into treatment groups. Each patient group is treated with weekly intravenous injections of fully human monoclonal antibodies against α5β1 as described herein. Each patient is dosed with an effective amount of the antibody ranging from 5 mg/kg/week to 20 mg/kg/week for 4-8 months. A control group is given only the standard chemotherapeutic regimen.

At periodic times during and after the treatment regimen, tumour burden is assessed by magnetic resonance imaging (MRI). It can be expected that the patients who have received weekly antibody treatments will show significant reductions in tumour size, time delay to progression or prolonged survival compared to patients that do not receive antibody treatment. In some treated patients, it can be expected that the tumours are no longer detectable. In contrast, it can be expected that tumour size increases or remains substantially the same in the control group.

### Example 19

### Inhibition of Colon Cancer in a Human Patient

A group of human cancer patients diagnosed with colon cancer is randomized into treatment groups. Each patient group is treated 3-weekly with intravenous injections of fully human monoclonal antibodies against α5β1 as described herein. Each patient is dosed with an effective amount of the antibody ranging from 5 mg/kg/week to 20 mg/kg/week for 4-8 months. A control group is given only the standard chemotherapeutic regimen. At periodic times during and after the treatment regimen, tumour burden is assessed by magnetic resonance imaging (MRI). It can be expected that the patients who have received 3-weekly antibody treatments show significant reductions in tumour size, time delay to progression or prolonged survival compared to patients that do not receive the antibody treatment. In some treated patients, it can be expected that the tumours are no longer detectable. In contrast, it can be expected that tumour size increases or remains substantially the same in the control group.

### Example 20

### Inhibition of Melanoma in a Human Patient

A group of human cancer patients diagnosed with melanoma is randomized into treatment groups. Each patient group is treated 3-weekly with intravenous injections of fully human monoclonal antibodies against α5β1 as described herein. Each patient is dosed with an effective amount of the antibody ranging from 5 mg/kg/week to 20 mg/kg/week for 4-8 months. A control group is given only the standard chemotherapeutic regimen. At periodic times during and after the treatment regimen, tumour burden is assessed by magnetic resonance imaging (MRI). It can be expected that the patients who have received 3-weekly antibody treatments with antibodies against α5β1 show significant reductions in melanoma, time delay to progression or prolonged survival compared to patients that do not receive the antibody treatment. In some treated patients, it can be expected that the melanoma lesions are no longer detectable. In contrast, it can be expected that melanoma increases or remains substantially the same in the control group.

### Example 21

### Inhibition of Chronic Myelogenous Leukemia (CML) in a Human Patient

A group of human cancer patients diagnosed with CML is randomized into treatment groups. Each patient group is treated 3-weekly with intravenous injections of fully human monoclonal antibodies against α5β1 as described herein. Each patient is dosed with an effective amount of the antibody ranging from 5 mg/kg/week to 20 mg/kg/week for 4-8 months. A control group is given only the standard chemotherapeutic regimen. At periodic times during and after the treatment regimen, tumour burden is assessed by magnetic resonance imaging (MRI). It can be expected that the patients who have received 3-weekly antibody treatments show significant reductions in CML, time delay to progression or prolonged survival compared to patients that do not receive the antibody treatment. In some treated patients, it can be expected that the CML is no longer detectable. In contrast, it can be expected that CML increases or remains substantially the same in the control group.

### Example 22

### Inhibition of Tumour Cell Growth in a Human Patient

A human patient is diagnosed with a malignant tumour. The patient is treated with weekly intravenous injections of fully human monoclonal antibodies against α5β1 as described herein for 8 weeks. At periodic times during and after the treatment regimen, tumour burden is assessed by magnetic resonance imaging (MRI). It can be expected that significant reductions in tumour size are found.

### Deposit of Biological Materials

A deposit of E. coli Top 10 containing a plasmid which encodes the 3C5 antibody light chain was made under the terms of the Budapest Treaty on Nov. 26, 2009 at the National Collections of Industrial and Marine Bacteria (NCIMB) Ltd., Ferguson Building, Craibstone Estate, Bucksburn, Aberdeen, Scotland, AB21 9YA, UK and has been assigned Accession No. 41668.

Likewise, a deposit of E. coli Top 10 containing a plasmid which encodes the 3C5 antibody heavy chain was made under the terms of the Budapest Treaty on Nov. 26, 2009 at the National Collections of Industrial and Marine Bacteria (NCIMB) Ltd., Ferguson Building, Craibstone Estate, Bucksburn, Aberdeen, Scotland, AB21 9YA, UK and has been assigned Accession No. 41669.

### EQUIVALENTS

The foregoing written specification is considered to be sufficient to enable one skilled in the art to practice the invention. The foregoing description and Examples detail certain preferred embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

1. A targeted binding agent that specifically binds to α5β1 integrin with a Kd of less than 100 picomolar.

2. A targeted binding agent of claim 1, wherein said targeted binding agent binds α5β1 integrin with a Kd of less than 40 picomolar.

3. A targeted binding agent of claim 1, wherein said targeted binding agent inhibits binding of fibronectin, fibrin, adhesion molecule L1-CAM, Tie-2 and/or Flt1 ligands to α5β1 integrin.

4. A targeted binding agent of claim 1 wherein said targeted binding agent comprises heavy and light chain variable regions according to Table 12 and 13.

5. A targeted binding agent of claim 1 wherein said targeted binding agent is MAb 3C5 or 5B11.

6. A targeted binding agent of claim 1, wherein said targeted binding agent comprises a polypeptide comprising the sequence of SEQ ID NO.: 22.

7. A targeted binding agent of claim 1, wherein said targeted binding agent comprises a polypeptide comprising the sequence of SEQ ID NO.: 24.

8. A targeted binding agent of claim 1, wherein said targeted binding agent comprises a polypeptide comprising the sequence of SEQ ID NO.: 48.

9. A targeted binding agent of claim 1, wherein said targeted binding agent comprises a polypeptide comprising the sequence of SEQ ID NO.: 50.

10. A targeted binding agent which competes for binding to α5β1 integrin with the targeted binding agent of claim 5.

11. A targeted binding agent comprising an amino acid sequence comprising:
a) a CDR3 sequence as shown in Table 12 or 13;
b) a CDR3 sequence as shown in Table 12 and a CDR3 sequence as shown in Table 13.
c) a CDR1, CDR2, and CDR3 sequence as shown in Table 12; or
d) a CDR1, a CDR2 and a CDR3 sequence as shown in Table 13; or
e) a CDR1, a CDR2 and a CDR3 sequence as shown in Table 12 and a CDR1, a CDR2 and a CDR3 sequence as shown in Table 13; or
f) a CDR1, CDR2 and CDR3 sequence of McAb 3C5 as shown in Table 12 and a CDR1, CDR2, CDR3 sequence of McAb 3C5 as shown in Table 13; or
g) a CDR1, CDR2 and CDR3 sequence of McAb 5B11 as shown in Table 12 and a CDR1, CDR2, CDR3 sequence of McAb 5B11 as shown in Table 13.

12. A targeted binding agent that specifically binds to α5β1 and integrin, comprising a set of CDRs: HCDR1, HCDR2, HCDR3, LCDR1, LCDR2, LCDR2, wherein the set of CDRs has 10 or fewer amino acid substitutions from a set of CDRs in which:
HCDR1 is amino acid sequence SEQ ID NO: 25;
HCDR2 is amino acid sequence SEQ ID NO: 26;
HCDR3 is amino acid sequence SEQ ID NO: 27;
LCDR1 is amino acid sequence SEQ ID NO: 28;
LCDR2 is amino acid sequence SEQ ID NO: 29; and
LCDR3 is amino acid sequence SEQ ID NO: 30.

13. A targeted binding agent that specifically binds to α5β1 integrin, comprising a set of CDRs: HCDR1, HCDR2, HCDR3, LCDR1, LCDR2, LCDR2, wherein the set of CDRs has 10 or fewer amino acid substitutions from a set of CDRs in which:
HCDR1 is amino acid sequence SEQ ID NO: 51;
HCDR2 is amino acid sequence SEQ ID NO: 52;
HCDR3 is amino acid sequence SEQ ID NO: 53;
LCDR1 is amino acid sequence SEQ ID NO: 54;
LCDR2 is amino acid sequence SEQ ID NO: 55; and
LCDR3 is amino acid sequence SEQ ID NO: 56.

14. A targeted binding agent of claim 1 wherein said targeted binding agent is a monoclonal antibody.

15. (canceled)

16. A targeted binding agent of claim 14, wherein said targeted binding agent is a binding fragment selected from the group consisting of Fab, Fab′, F(ab′)₂, Fv, ScFv, ScFvFc and dAb.

17. A nucleic acid encoding a targeted binding agent according to claim 1.

18. A vector comprising the nucleic acid molecule of claim 17.

19. A host cell comprising the vector of claim 18.

20. A method of producing an antibody comprising culturing the host cell of claim 19 and recovering the antibody from the cell culture.

21. A method of treating a neoplastic disease in a mammal comprising:
selecting an animal in need of treatment for a neoplastic disease; and administering to said animal a therapeutically effective dose of a targeted binding agent of claim 1.

22. The method of claim 21, wherein said neoplastic disease is selected from the group consisting of: melanoma, small cell lung cancer, non-small cell lung cancer, glioma, hepatocellular carcinoma, thyroid tumour, gastric cancer, prostate cancer, breast cancer, ovarian cancer, bladder cancer, lung cancer, glioblastoma, endometrial cancer, kidney cancer, colon cancer, pancreatic cancer, esophageal carcinoma, head and neck cancers, mesothelioma, sarcomas, biliary, small bowel adenocarcinoma, pediatric malignancies, epidermoid carcinoma and gastrointestinal stromal tumour.

23. A method of treating a non-neoplastic disease in a mammal comprising:
selecting an animal in need of treatment for a non-neoplastic disease; and
administering to said animal a therapeutically effective dose of a targeted binding agent of claim 1.

24. The method of claim 23, wherein said non-neoplastic disease is selected from the group consisting of: ocular disease, inflammatory disease, cardiovascular disease and sepsis.

25-27. (canceled)

28. A targeted binding agent of claim 1 in association with a pharmaceutically acceptable carrier.

29. A method of antagonizing α5β1 integrin signaling with a targeted binding agent of claim 1 in combination with an antagonist of vascular endothelial growth factor.

30. A targeted binding agent of claim 1 comprising an amino acid sequence comprising:
a) a CDR3 sequence as shown in Table 12 or 13;
b) a CDR3 sequence as shown in Table 12 and a CDR3 sequence as shown in Table 13,
c) a CDR1, CDR2, and CDR3 sequence as shown in Table 12; or
d) a CDR1, a CDR2 and a CDR3 sequence as shown in Table 13; or
e) a CDR1, a CDR2 and a CDR3 sequence as shown in Table 12 and a CDR1, a CDR2 and a CDR3 sequence as shown in Table 13; or
f) a CDR1, CDR2 and CDR3 sequence of McAb 3C5 as shown in Table 12 and a CDR1, CDR2, CDR3 sequence of McAb 3C5 as shown in Table 13; or
g) a CDR1, CDR2 and CDR3 sequence of McAb 5B11 as shown in Table 12 and a CDR1, CDR2, CDR3 sequence of McAb 5B11 as shown in Table 13.
